(19) United States
(12) Patent Application Publication
(10) Pub. No.: US 2005/0032693 A1
(43) Pub. Date: Feb. 10, 2005

(54) Novel polypeptides, their nucleic acids, and methods for their use in angiogenesis and vascularization

(75) Inventors: Mary E. Gerritsen, San Mateo, CA (US); Audrey Goddard, San Francisco, CA (US); J. Christopher Grimaldi, San Francisco, CA (US); Fuad Mehraban, Trumbull, CT (US)

Correspondence Address: MERCHANT & GOULD PC, P.O. BOX 2903, MINNEAPOLIS, MN, 55402-0903, US

(73) Assignees: Genentech, Inc., South San Francisco, CA (US); Curagen Corporation, New Haven, CT (US)

(21) Appl. No.: 10/811,080

(22) Filed: Mar. 26, 2004

Related U.S. Application Data
(63) Continuation of application No. 09/684,458, filed on Oct. 5, 2000, now abandoned.
(60) Provisional application No. 60/158,587, filed on Oct. 7, 1999.
(60) Provisional application No. 60/162,611, filed on Oct. 28, 1999.

(51) Int. Cl. A61K 38/17; C12Q 1/68; C07H 21/04

(52) U.S. Cl. 514/12; 530/350; 536/23.5; 435/6; 435/69.1; 435/320.1; 435/325

(57) ABSTRACT

The present invention is directed to novel polypeptides critical for angiogenesis and vascularization, and to nucleic acid molecules encoding those polypeptides. Also provided herein are vectors and host cells comprising those nucleic acid sequences, chimeric polypeptide molecules comprising the polypeptides of the present invention fused to heterologous polypeptide sequences, antibodies which bind to the polypeptides of the present invention and to methods for producing the polypeptides of the present invention. Compositions and methods are disclosed for stimulating or inhibiting angiogenesis and/or neo- or cardio-vascularization in mammals, including humans. Pharmaceutical compositions are based on polypeptides or antagonists thereto that have been identified for one or more of these uses. Disorders that can be diagnosed, prevented, or treated by the compositions herein include trauma such as wounds; various cancers, and disorders of the vessels including atherosclerosis.

### FIELD OF THE INVENTION

The present invention relates generally to the identification and isolation of novel DNA and their encoded intracellular polypeptides designated herein as “PRO-C-MG.2, PRO-C-MG.12, PRO-C-MG.45, PRO-C-MG.64 or PRO-C-MG.72” polypeptides, whose gene expression is modulated in cells undergoing angiogenesis and/or vascularization. Accordingly, the present invention further relates to compositions and methods useful for promoting or inhibiting angiogenesis and/or neo- or cardio-vascularization in mammals in need of such biological effect. This includes the diagnosis and treatment of cardiovascular disorders as well as oncological disorders.

### BACKGROUND OF THE INVENTION

Intracellular proteins play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, recognized by and activate diverse cell receptors or membrane-bound proteins. Each activation signal initiates a specific, signal transduction pathway composed of intracellular proteins (e.g., protein kinases, DNA-binding regulatory proteins, protein processing proteins, proteases, glycosidases) resulting in the modulation, either up- or down-regulation, of the activity, expression, or amount of other intracellular proteins involved in or necessary for the cell's fate in response to the signal. For example, detectable changes in the RNA or protein levels of intracellular proteins necessary for cell growth or differentiation in response to appropriate transduction of signals can be controlled in part by receptor-mediated phosphorylation of signal-induction-pathway related intracellular proteins.

Intracellular proteins and their gene sequences have various industrial applications, including as drug targets for pharmaceuticals, diagnostics, pharmaceuticals, biosensors, and bioreactors. While most protein drugs available at present are secreted cytokines or their antibody mimics, most targets of small molecule, peptide, or antisense drugs are intracellular proteins or the intracellular genes that encode them. For example, such drugs can interact with an intracellular protein target to block its activity and disrupt the related signal transduction pathway, thereby stopping (or modulating) the cell's response or activity controlled by that pathway. Both industry and academia are undertaking efforts to identify new, native intracellular proteins and their genes, the signal transduction pathways in which they function, and the proteins or genes they modulate. Classically, such genes and their proteins are discovered by binary comparison studies in which a differential analysis is made of RNA or protein upon a cell or tissue response to a certain stimulus.

One consequence of cellular response is the formation of new blood vessels, which can occur by two related mechanisms: angiogenesis-the growth of new vessels from pre-existing vessels—and vasculogenesis—the formation of vessels through aggregation of endothelial cells. All blood vessel inner surfaces are lined with endothelial cells. Vascular endothelial cells, at the interface between blood and extravascular space, play prominent roles in maintaining cardiovascular homeostasis and mediate pathophysiologic responses to injury. For example, angiogenesis occurs in the adult during events such as wound healing and ovulation. During angiogenesis, endothelial cells responding to environmental stimuli undergo a number of cellular alterations and responses, resulting in a complex series of steps, which involve degradation of the basement membrane by cellular proteases, penetration and migration of endothelial cells into the extracellular matrix, endothelial proliferation, and the formation of interconnected vascular networks. This formation of new vessels takes place in distinct phases that entails and relies upon modulation or expression of a variety of intracellular proteins, extracellular matrix components, proteases an protease inhibitors, inflammatory molecules, chemokines, and molecules involved in cell division and proliferation, cytoskeletal rearrangement, adhesion molecules and also apoptosis of certain endothelial cell populations.

Endothelial cells also undergo angiogenesis during the neovascularization associated with tumor growth and metastasis and a variety of non-neoplastic diseases or disorders. In the case of tumor growth, angiogenesis appears to be crucial for the transition from hyperplasia to neoplasia, and for providing nourishment to the growing solid tumor (Folkman, et al., Nature 339:58 (1989)). Angiogenesis allows tumors to be in contact with the vascular bed of the host, which provides a route for metastasis of the tumor cells. In fact, the progression of solid tumor growth and metastasis depends on angiogenesis, as supported for example, by studies showing a correlation between the number and density of microvessels in histologic sections of invasive human breast carcinoma and actual presence of distant metastases (Weidner, et al., New Engl. J. Med., 324:1 (1991)). Recent data suggests that blocking new blood vessel growth can slow tumor growth by cutting off the supply of oxygen and nutrients; without a new blood supply tumors cannot grow more than about 1-2 mm in diameter. Thus new angiostatic therapies to treat cancer are desired.

There exists a need for additional products, methods and assays that provide a means to control signal transduction pathways and thereby modulate cellular and tissue response and activity. Such products, methods and assays will provide benefit in numerous medical conditions and procedures.

In view of the role of vascular endothelial cell growth and angiogenesis in many diseases and disorders, it is desirable to have a means of modulating one or more of the biological effects causing these processes, in order to provide benefits such as enhancing repair or maintenance of blood vessels and reducing or inhibiting cancer and tumor progression. It is also desirable to have a means of assaying for the presence of pathogenic polypeptides in normal and diseased conditions, and especially cancer. Further, as there is no generally applicable therapy for the treatment of cardiac hypertrophy, the identification of factors that can prevent or reduce cardiac myocyte hypertrophy is of primary importance in the development of new therapeutic strategies to inhibit pathophysiological cardiac growth.

While there are several treatment modalities for various cardiovascular and oncologic disorders, there is still a need for additional therapeutic approaches. As a further means to address these existing needs, the identification and characterization of novel intracellular polypeptides designated herein as “PRO-C-MG.2, PRO-C-MG.12, PRO-C-MG.45, PRO-C-MG.64 or PRO-C-MG.72” polypeptides are provided.

### SUMMARY OF THE INVENTION

cDNA clones, designated herein as DNA-C-MG.2-1776, DNA-C-MG.12-1776, DNA-C-MG.45-1776, DNA-C-MG.64-1776 or DNA-C-MG.72-1776, that encode a novel polypeptide designated in the present application as “PRO-C-MG.2, PRO-C-MG.12, PRO-C-MG.45, PRO-C-MG.64 or PRO-C-MG.72”, respectively, have been identified, whose DNA-C-MG.2-1776, DNA-C-MG.12-1776, DNA-C-MG.45-1776, DNA-C-MG.64-1776 or DNA-C-MG.72-1776 RNA is modulated in cells undergoing tube formation by endothelial cells, which is a necessary step in the development of a blood vessel during angiogenesis and vasculogenesis. Differential cDNA screening, GeneCalling™ technology, was applied to human umbilical cord endothelial cells (HUVECS) undergoing tube formation in collagen gels in the presence of growth factors, mimicking the angiogenic environment of endothelial cells in vivo. The three dimensional gel is pre-requisite for the differentiation and fusion of endothelial cells into tubes; HUVECS grown on the surface of gelatin or on plastic do not undergo tube-formation.

Accordingly, the present invention concerns compositions and methods for promoting or inhibiting angiogenesis and/or vascularization, preferably neo- or cardio-vascularization in mammals, and for identifying additional molecules providing that benefit. The molecules of the present invention are believed to be useful drugs for the diagnosis and/or treatment (including prevention) of disorders where such effects are desired, such as the promotion or inhibition of angiogenesis, inhibition or stimulation of vascular endothelial cell growth, stimulation of growth or proliferation of vascular endothelial cells, inhibition of tumor growth, inhibition of angiogenesis-dependent tissue growth, stimulation of angiogenesis-dependent tissue growth, inhibition of cardiac hypertrophy and stimulation of cardiac hypertrophy, e.g., for the treatment of congestive heart failure. The present invention provides methods for promoting or inhibiting angiogenesis by supplying to endothelial tissue an effective amount of a compound of the invention. Also provided are methods for treating a tumor, reducing the size of a tumor, reducing the vasculature supporting a tumor or reducing the tumor burden of a mammal by administering an effective amount of a compound of the invention.

In one embodiment, the invention provides an isolated nucleic acid molecule comprising a nucleotide sequence that encodes a PRO-C-MG.2, PRO-C-MG.12, PRO-C-MG.45, PRO-C-MG.64 or PRO-C-MG.72 polypeptide.

In one embodiment, the isolated nucleic acid molecule comprises a nucleotide sequence having at least about 80% sequence identity, with increasing preference for each one percent increase in sequence identity, to at least about 99% sequence identity to (a) a DNA molecule encoding a PRO-C-MG.2, PRO-C-MG.12, PRO-C-MG.45, PRO-C-MG.64 or PRO-C-MG.72 polypeptide having the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) the complement of the DNA molecule of (a), or a DNA molecule encoding the PRO-C-MG.2, PRO-C-MG.12, PRO-C-MG.45, PRO-C-MG.64 or PRO-C-MG.72 polypeptide encoded by the ATCC-deposited DNA of the invention as described herein.

In another embodiment, the isolated nucleic acid molecule comprises (a) a nucleotide sequence encoding a PRO-C-MG.2, PRO-C-MG.12, PRO-C-MG.45, PRO-C-MG.64 or PRO-C-MG.72 polypeptide having the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) the complement of the nucleotide sequence of (a), or the ATCC-deposited DNA encoding a PRO-C-MG.2, PRO-C-MG.12, PRO-C-MG.45, PRO-C-MG.64 or PRO-C-MG.72 polypeptide.

In other embodiments, the isolated nucleic acid molecule comprises a nucleotide sequence having at least about 80% sequence identity, with increasing preference for each one percent increase in sequence identity, to at least about 99% sequence identity to (a) a DNA molecule having the sequence of nucleotides from about 66 to about 1796 of SEQ ID NO:1, about 465 to about 1886 of SEQ ID NO:3, about 271 to about 1788 of SEQ ID NO:17, about 267 to about 1298 of SEQ ID NO:15, or about 71 to about 2059 of SEQ ID NO:13, respectively, (b) the complement of the DNA molecule of (a), or the ATCC-deposited DNA encoding a PRO-C-MG.2, PRO-C-MG.12, PRO-C-MG.45, PRO-C-MG.64 or PRO-C-MG.72 polypeptide.

In another embodiment, the isolated nucleic acid molecule comprises (a) the nucleotide sequence of from about 66 to about 1796 of SEQ ID NO: 1, about 465 to about 1886 of SEQ ID NO:3, about 271 to about 1788 of SEQ ID NO:17, about 267 to about 1298 of SEQ ID NO:15, or about 71 to about 2059 of SEQ ID NO:13, respectively, or (b) the complement of the nucleotide sequence of (a), or the deposited DNA encoding a PRO-C-MG.2, PRO-C-MG.12, PRO-C-MG.45, PRO-C-MG.64 or PRO-C-MG.72 polypeptide.

In another embodiment, the invention concerns an isolated nucleic acid molecule which encodes an active PRO-C-MG.2, PRO-C-MG.12, PRO-C-MG.45, PRO-C-MG.64 or PRO-C-MG.72 polypeptide as defined herein comprising a nucleotide sequence that hybridizes to the complement of a nucleic acid sequence that encodes amino acids about 1 to about 577 of SEQ ID NO:2, about 1 to about 474 of SEQ ID NO: 4, about 1 to about 506 of SEQ ID NO: 18. about 1 to about 344 of SEQ ID NO: 16, or about 1 to about 633 of SEQ ID NO: 14, respectively. Preferably, hybridization occurs under stringent hybridization and wash conditions.

In yet another embodiment, the invention concerns an isolated nucleic acid molecule which encodes an active PRO-C-MG.2, PRO-C-MG.12, PRO-C-MG.45, PRO-C-MG.64 or PRO-C-MG.72 polypeptide as defined herein comprising a nucleotide sequence that hybridizes to the complement of the nucleic acid sequence between about 66 to about 1796 of SEQ ID NO:1, about 465 to about 1886 of SEQ ID NO:3, about 271 to about 1788 of SEQ ID NO:17, about 267 to about 1298 of SEQ ID NO:15, or about 71 to about 2059 of SEQ ID NO:13, respectively. Preferably, hybridization occurs under stringent hybridization and wash conditions.

In a further embodiment, the invention concerns an isolated nucleic acid molecule which is produced by hybridizing a test DNA molecule under stringent conditions with (a) a DNA molecule encoding a PRO-C-MG.2, PRO-C-MG.12, PRO-C-MG.45, PRO-C-MG.64 or PRO-C-MG.72 polypeptide having the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) the complement of the DNA molecule of (a), and, if the test DNA molecule has at least about an 80% sequence identity, with increasing preference for each one percent increase in sequence identity, to at least about 99% sequence identity to (a) or (b), and isolating the test DNA molecule. Such a molecule, hybridizable to the DNA molecule encoding a PRO-C-MG.2, PRO-C-MG.12, PRO-C-MG.45, PRO-C-MG.64 or PRO-C-MG.72 polypeptide having the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, have at least about 596 and 1535 nucleotides, respectively.

In another embodiment, the invention concerns an isolated PRO-C-MG.2, PRO-C-MG.12, PRO-C-MG.45, PRO-C-MG.64 or PRO-C-MG.72 nucleic acid molecule comprising (a) a nucleotide sequence encoding a polypeptide scoring at least about 80% positives, with increasing preference for each one percent increase in positives, to at least about 99% positives when compared with the amino acid sequence of residues about 1 to about 577 of SEQ ID NO:2 or about 1 to about 474 of SEQ ID NO:4, respectively, or (b) the complement of the nucleotide sequence of (a).

Another embodiment is directed to fragments of a PRO-C-MG.2, PRO-C-MG.12, PRO-C-MG.45, PRO-C-MG.64 or PRO-C-MG.72 polypeptide coding sequence that can find use as, for example, hybridization probes or for encoding fragments of a PRO-C-MG.2, PRO-C-MG.12, PRO-C-MG.45, PRO-C-MG.64 or PRO-C-MG.72 polypeptide that can optionally encode a polypeptide comprising a binding site for an anti-PRO-C-MG.2, PRO-C-MG.12, PRO-C-MG.45, PRO-C-MG.64 or PRO-C-MG.72 binding target, preferably an antibody, a natural PRO-C-MG.2, PRO-C-MG.12, PRO-C-MG.45, PRO-C-MG.64 or PRO-C-MG.72 intracellular binding target, or a nonnatural binding agent. Such nucleic acid fragments are usually at least about 20 nucleotides in length with increasing preference to at least about 1000 nucleotides in length, wherein in this context the term “about” means the referenced nucleotide sequence length plus or minus 10% of that referenced length. In a preferred embodiment, the nucleotide sequence fragment is derived from any coding region of the nucleotide sequence shown in SEQ ID NO:1 or SEQ ID NO:3. Also contemplated are the PRO-C-MG.2, PRO-C-MG.12, PRO-C-MG.45, PRO-C-MG.64 or PRO-C-MG.72 polypeptide fragments encoded by these nucleotide molecule fragments, preferably those PRO-C-MG.2, PRO-C-MG.12, PRO-C-MG.45, PRO-C-MG.64 or PRO-C-MG.72 polypeptide fragments that comprise a binding site for an anti-PRO-C-MG.2, PRO-C-MG.12, PRO-C-MG.45, PRO-C-MG.64 or PRO-C-MG.72 binding target, preferably an antibody, a natural PRO-C-MG.2, PRO-C-MG.12, PRO-C-MG.45, PRO-C-MG.64 or PRO-C-MG.72 intracellular binding target, or a nonnatural binding agent.

In another embodiment, the invention provides a vector comprising a nucleotide sequence encoding PRO-C-MG.2, PRO-C-MG.12, PRO-C-MG.45, PRO-C-MG.64 or PRO-C-MG.72 or variants thereof. The vector can comprise any of the isolated nucleic acid molecules identified herein.

A host cell comprising such a vector is also provided. The host cells can be vertebrate, mammalian, fungal, plant, or bacterial cells. Preferred are yeast cells, CHO cells, E. coli, yeast, human or mouse cells. A process for producing PRO-C-MG.2, PRO-C-MG.12, PRO-C-MG.45, PRO-C-MG.64 or PRO-C-MG.72 polypeptides is further provided and comprises culturing host cells under conditions suitable for expression of PRO-C-MG.2, PRO-C-MG.12, PRO-C-MG.45, PRO-C-MG.64 or PRO-C-MG.72 in order to produce the PRO-C-MG.2, PRO-C-MG.12, PRO-C-MG.45, PRO-C-MG.64 or PRO-C-MG.72 polypeptide. In a further embodiment, the PRO-C-MG.2, PRO-C-MG.12, PRO-C-MG.45, PRO-C-MG.64 or PRO-C-MG.72 polypeptide can be recovered from the cell culture. As used throughout, “cell culture” includes the cells or cell medium.

In another embodiment, the invention provides isolated PRO-C-MG.2, PRO-C-MG.12, PRO-C-MG.45, PRO-C-MG.64 or PRO-C-MG.72 polypeptide encoded by any of the isolated nucleic acid sequences identified herein.

In a specific embodiment, the invention provides isolated native sequence PRO-C-MG.2, PRO-C-MG.12, PRO-C-MG.45, PRO-C-MG.64 or PRO-C-MG.72 polypeptide, which in certain embodiments, includes an amino acid sequence comprising residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively.

In another embodiment, the invention concerns an isolated PRO-C-MG.2, PRO-C-MG.12, PRO-C-MG.45, PRO-C-MG.64 or PRO-C-MG.72 polypeptide, comprising an amino acid sequence having at least about 80% sequence identity, with increasing preference for each one percent increase in sequence identity, to at least about 99% sequence identity to the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively.

In a further embodiment, the invention concerns an isolated PRO-C-MG.2, PRO-C-MG.12, PRO-C-MG.45, PRO-C-MG.64 or PRO-C-MG.72 polypeptide comprising an amino acid sequence having at least about 80% sequence identity, with increasing preference for each one percent increase in sequence identity, to at least about 99% sequence identity to an amino acid sequence encoded by the human protein cDNA deposited with the ATCC as described herein.

In a further embodiment, the invention concerns an isolated PRO-C-MG.2, PRO-C-MG.12, PRO-C-MG.45, PRO-C-MG.64 or PRO-C-MG.72 polypeptide comprising an amino acid sequence scoring at least about 80% positives, with increasing preference for each one percent increase in positives, to at least about 99% positives when compared with the amino acid sequence of residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14. respectively.

In yet another embodiment, the invention concerns an isolated PRO-C-MG.2, PRO-C-MG.12, PRO-C-MG.45, PRO-C-MG.64 or PRO-C-MG.72 polypeptide, comprising the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or a fragment thereof which is biologically active or sufficient to provide a binding site for an anti-PRO-C-MG.2, PRO-C-MG.12, PRO-C-MG.45, PRO-C-MG.64 or PRO-C-MG.72 binding target, preferably an antibody, a natural PRO-C-MG.2, PRO-C-MG.12, PRO-C-MG.45, PRO-C-MG.64 or PRO-C-MG.72 intracellular binding target, or a nonnatural binding agent, wherein the identification of PRO-C-MG.2, PRO-C-MG.12, PRO-C-MG.45, PRO-C-MG.64 or PRO-C-MG.72 polypeptide fragments that possess biological activity or provide the binding site can be accomplished in a routine manner using techniques which are well known in the art. Preferably, the PRO-C-MG.2, PRO-C-MG.12, PRO-C-MG.45, PRO-C-MG.64 or PRO-C-MG.72 fragment retains a qualitative biological activity of a native PRO-C-MG.2, PRO-C-MG.12, PRO-C-MG.45, PRO-C-MG.64 or PRO-C-MG.72 polypeptide.

In a still further embodiment, the invention provides a polypeptide produced by (i) hybridizing a test DNA molecule under stringent conditions with (a) a DNA molecule encoding a PRO-C-MG.2, PRO-C-MG.12, PRO-C-MG.45, PRO-C-MG.64 or PRO-C-MG.72 polypeptide having the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) the complement of the DNA molecule of (a), and if the test DNA molecule has at least about an 80% sequence identity, with increasing preference for each one percent increase in sequence identity, to at least about 99% sequence identity to (a) or (b), (ii) culturing a host cell comprising the test DNA molecule under conditions suitable for expression of the polypeptide in order to produce the polypeptide, and then optionally (iii) recovering the polypeptide from the cell culture.

In another embodiment, the invention provides chimeric molecules comprising a PRO-C-MG.2, PRO-C-MG.12, PRO-C-MG.45, PRO-C-MG.64 or PRO-C-MG.72 polypeptide fused to a heterologous polypeptide or amino acid sequence, wherein the PRO-C-MG.2, PRO-C-MG.12, PRO-C-MG.45, PRO-C-MG.64 or PRO-C-MG.72 polypeptide can comprise any PRO-C-MG.2, PRO-C-MG.12, PRO-C-MG.45, PRO-C-MG.64 or PRO-C-MG.72 polypeptide, variant or fragment thereof as described herein. An example of such a chimeric molecule comprises a PRO-C-MG.2, PRO-C-MG.12, PRO-C-MG.45, PRO-C-MG.64 or PRO-C-MG.72 polypeptide fused to an epitope tag sequence, a Fc region of an immunoglobulin, or a secretion signal peptide.

In one embodiment, the present invention provides a composition comprising a PRO-C-MG.2, PRO-C-MG.12, PRO-C-MG.45, PRO-C-MG.64 or PRO-C-MG.72 polypeptide in admixture with a pharmaceutically acceptable carrier. In one aspect, the composition comprises a therapeutically effective amount of the polypeptide. In another aspect, the composition comprises a further active ingredient, namely, a cardiovascular, endothelial or angiogenic agent or an angiostatic agent, preferably an angiogenic or angiostatic agent. Preferably, the composition is sterile.

In a further embodiment, the present invention provides a method for preparing such a composition useful for the treatment of a cardiovascular, endothelial or angiogenic disorder comprising admixing a therapeutically effective amount of a PRO-C-MG.2, PRO-C-MG.12, PRO-C-MG.45, PRO-C-MG.64 or PRO-C-MG.72 polypeptide with a pharmaceutically acceptable carrier.

In another embodiment, the invention provides an antibody as defined herein which specifically binds to a PRO-C-MG.2, PRO-C-MG.12, PRO-C-MG.45, PRO-C-MG.64 or PRO-C-MG.72 polypeptide as described herein. Optionally, the antibody is a monoclonal antibody, an antibody fragment or a single chain antibody.

In yet another embodiment, the invention concerns agonists and antagonists of a native PRO-C-MG.2, PRO-C-MG.12, PRO-C-MG.45, PRO-C-MG.64 or PRO-C-MG.72 polypeptide as defined herein. Preferably, the agonist or antagonist is a molecule that modulates PRO-C-MG.2, PRO-C-MG.12, PRO-C-MG.45, PRO-C-MG.64 or PRO-C-MG.72 biological activity by acting at the post-translational, translational, transcriptional, or translocational level. In a particular embodiment the agonist or antagonist is an anti-PRO-C-MG.2, PRO-C-MG.12, PRO-C-MG.45, PRO-C-MG.64 or PRO-C-MG.72 antibody, an antigene molecule (sense or antisense), a PRO-C-MG.2, PRO-C-MG.12, PRO-C-MG.45, PRO-C-MG.64 or PRO-C-MG.72 gene (e.g. for gene therapy) or a small molecule.

In one such embodiment are therapeutic PRO-C-MG.2, PRO-C-MG.12, PRO-C-MG.45, PRO-C-MG.64 or PRO-C-MG.72 nucleic acids that are used to modulate cellular expression or intracellular concentration or availability of active PRO-C-MG.2, PRO-C-MG.12, PRO-C-MG.45, PRO-C-MG.64 or PRO-C-MG.72. These nucleic acids include antigene compounds, more typically antisense: single-stranded sequences comprising complements of the disclosed PRO-C-MG.2, PRO-C-MG.12, PRO-C-MG.45, PRO-C-MG.64 or PRO-C-MG.72 nucleic acids, and also include nucleic acid expressing PRO-C-MG.2 and PRO-C-MG.12 for gene therapy. Antigene modulation of PRO-C-MG.2, PRO-C-MG.12, PRO-C-MG.45, PRO-C-MG.64 or PRO-C-MG.72 expression can employ PRO-C-MG.2, PRO-C-MG.12, PRO-C-MG.45, PRO-C-MG.64 or PRO-C-MG.72 antisense nucleic acids operably linked to gene regulatory sequences. Cell are transfected with a vector comprising an PRO-C-MG.2, PRO-C-MG.12, PRO-C-MG.45, PRO-C-MG.64 or PRO-C-MG.72 sequence with a promoter sequence oriented such that transcription of the gene yields an antisense transcript capable of binding to endogenous PRO-C-MG.2, PRO-C-MG.12, PRO-C-MG.45, PRO-C-MG.64 or PRO-C-MG.72 encoding mRNA. Transcription of the antisense nucleic acid may be constitutive or inducible and the vector may provide for stable extrachromosomal maintenance or integration. In yet another embodiment, single-stranded antigene nucleic acids that bind to genomic DNA or RNA encoding a PRO-C-MG.2, PRO-C-MG.12, PRO-C-MG.45, PRO-C-MG.64 or PRO-C-MG.72 are administered to the target cell, in or temporarily isolated from a host, at a concentration that results in a substantial reduction in PRO-C-MG.2, PRO-C-MG.12, PRO-C-MG.45, PRO-C-MG.64 or PRO-C-MG.72 expression.

In one embodiment provided are PRO-C-MG.2, PRO-C-MG.12, PRO-C-MG.45, PRO-C-MG.64 or PRO-C-MG.72 compounds that have one or more PRO-C-MG.2, PRO-C-MG.12, PRO-C-MG.45, PRO-C-MG.64 or PRO-C-MG.72-specific binding affinities, including the ability to specifically bind at least one natural human intracellular PRO-C-MG.2, PRO-C-MG.12, PRO-C-MG.45, PRO-C-MG.64 or PRO-C-MG.72-specific binding target or a binding agent such as a anti-PRO-C-MG.2, PRO-C-MG.12, PRO-C-MG.45, PRO-C-MG.64 or PRO-C-MG.72-specific antibody or agent identified in assays as described herein. Natural PRO-C-MG.2, PRO-C-MG.12, PRO-C-MG.45, PRO-C-MG.64 or PRO-C-MG.72 binding targets are readily identified by screening cells. membranes and cellular extracts and fractions with the disclosed materials and methods. For example, two-hybrid screening using PRO-C-MG.2, PRO-C-MG.12, PRO-C-MG.45, PRO-C-MG.64 or PRO-C-MG.72 fragments are used to identify intracellular targets which specifically bind such fragments.

In another embodiment, the present invention provides a composition comprising an agonist or antagonist of a PRO-C-MG.2, PRO-C-MG.12, PRO-C-MG.45, PRO-C-MG.64 or PRO-C-MG.72 polypeptide in admixture with a pharmaceutically acceptable carrier. In one aspect, the composition comprises a therapeutically effective amount of the agonist or antagonist. In another aspect, the composition comprises a further active ingredient, namely, a cardiovascular, endothelial or angiogenic agent or an angiostatic agent, preferably an angiogenic or angiostatic agent.

In a further embodiment, the present invention provides a method for preparing such a composition useful for the treatment of a cardiovascular, endothelial or angiogenic disorder comprising admixing a therapeutically effective amount of a PRO-C-MG.2, PRO-C-MG.12, PRO-C-MG.45, PRO-C-MG.64 or PRO-C-MG.72 polypeptide agonist or antagonist with a pharmaceutically acceptable carrier.

In one embodiment, the invention provides efficient methods of identifying compounds active at the level of a PRO-C-MG.2, PRO-C-MG.12, PRO-C-MG.45, PRO-C-MG.64 or PRO-C-MG.72 modulatable cellular function. Generally, these screening methods involve assaying for compounds which modulate a PRO-C-MG.2, PRO-C-MG.12, PRO-C-MG.45, PRO-C-MG.64 or PRO-C-MG.72 interaction with a natural PRO-C-MG.2, PRO-C-MG.12, PRO-C-MG.45, PRO-C-MG.64 or PRO-C-MG.72 binding target. The methods are amenable to automated, cost-effective high throughput screening of chemical libraries for lead compounds. Assays for binding agents are provided including protein-protein binding assays, immunoassays, and cell based assays. A preferred assay is a high-through put cell-based or in vitro binding assay. For example, the PRO-C-MG.2, PRO-C-MG.12, PRO-C-MG.45, PRO-C-MG.64 or PRO-C-MG.72 compositions can be part of a fusion product with another peptide or polypeptide, e.g. a polypeptide that is capable of providing or enhancing protein-protein binding, stability under assay conditions, or a tag for detection or anchoring. The assay mixtures can contain a natural intracellular PRO-C-MG.2, PRO-C-MG.12, PRO-C-MG.45, PRO-C-MG.64 or PRO-C-MG.72 binding target, or active portion thereof. The assay mixture can also contain a candidate pharmacological agent. The resultant mixture is incubated under conditions where, but for the presence of the candidate pharmacological agent, the PRO-C-MG.2, PRO-C-MG.12, PRO-C-MG.45, PRO-C-MG.64 or PRO-C-MG.72 specifically binds the cellular binding target, portion or analog with a reference binding affinity. A detected difference in the binding affinity of the PRO-C-MG.2, PRO-C-MG.12, PRO-C-MG.45, PRO-C-MG.64 or PRO-C-MG.72 protein to the target in the absence of the agent as compared with the binding affinity in the presence of the agent indicates that the agent modulates the binding of the PRO-C-MG.2, PRO-C-MG.12, PRO-C-MG.45, PRO-C-MG.64 or PRO-C-MG.72 protein to the PRO-C-MG.2, PRO-C-MG.12, PRO-C-MG.45, PRO-C-MG.64 or PRO-C-MG.72 binding target. Analogously, in a cell-based transcription assay, a difference in the PRO-C-MG.2, PRO-C-MG.12, PRO-C-MG.45, PRO-C-MG.64 or PRO-C-MG.72 transcriptional induction in the presence and absence of an agent indicates the agent modulates PRO-C-MG.2, PRO-C-MG.12, PRO-C-MG.45, PRO-C-MG.64 or PRO-C-MG.72-induced transcription. A difference, as used herein, is statistically significant and preferably represents at least a 50%, more preferably at least a 90% difference.

In a further embodiment, the invention concerns a method of identifying agonists or antagonists to a PRO-C-MG.2, PRO-C-MG.12, PRO-C-MG.45, PRO-C-MG.64 or PRO-C-MG.72 polypeptide which comprises contacting either the PRO-C-MG.2, PRO-C-MG.12, PRO-C-MG.45, PRO-C-MG.64 or PRO-C-MG.72 polypeptide, a cell comprising the PRO-C-MG.2, PRO-C-MG.12, PRO-C-MG.45, PRO-C-MG.64 or PRO-C-MG.72 polypeptide, or a PRO-C-MG.2, PRO-C-MG.12, PRO-C-MG.45, PRO-C-MG.64 or PRO-C-MG.72 nucleic acid with a candidate molecule and monitoring the specific binding to the PRO-C-MG.2, PRO-C-MG.12, PRO-C-MG.45, PRO-C-MG.64 or PRO-C-MG.72 polypeptide or nucleic acid and/or monitoring a biological activity mediated by the PRO-C-MG.2, PRO-C-MG.12, PRO-C-MG.45, PRO-C-MG.64 or PRO-C-MG.72 polypeptide. Preferably, the PRO-C-MG.2, PRO-C-MG.12, PRO-C-MG.45, PRO-C-MG.64 or PRO-C-MG.72 polypeptide is a native PRO-C-MG.2, PRO-C-MG.12, PRO-C-MG.45, PRO-C-MG.64 or PRO-C-MG.72 polypeptide.

In another embodiment, the present invention provides a method for identifying an agonist of a PRO-C-MG.2, PROC-MG.12, PRO-C-MG.45, PRO-C-MG.64 or PRO-C-MG.72 polypeptide comprising: (a) contacting target cells and a test compound to be screened under conditions suitable for the induction, stimulation or dependence of a cellular response normally induced by, stimulated by or dependent on a PRO-C-MG.2, PRO-C-MG.12, PRO-C-MG.45, PRO-C-MG.64 or PRO-C-MG.72 polypeptide; and (b) determining the induction, stimulation or dependence of the cellular response to determine if the test compound is an effective agonist, wherein the induction or enhancement of the cellular response is indicative of the test compound being an effective agonist. In a preferred embodiment, the target cells have been engineered or treated to prevent expressing endogenous PRO-C-MG.2, PRO-C-MG.12, PRO-C-MG.45, PRO-C-MG.64 or PRO-C-MG.72 during the test period. The cellular response is preferably cell proliferation or tube formation.

In another embodiment, the present invention provides a method for identifying an antagonist of a PRO-C-MG.2, PRO-C-MG.12, PRO-C-MG.45, PRO-C-MG.64 or PRO-C-MG.72 polypeptide comprising: (a) contacting target cells and a test compound to be screened under conditions suitable for the induction, stimulation or dependence of a cellular response normally induced by, stimulated by or dependent on a PRO-C-MG.2, PRO-C-MG.12, PRO-C-MG.45, PRO-C-MG.64 or PRO-C-MG.72 polypeptide; and (b) determining the induction, stimulation or dependence of the cellular response to determine if the test compound is an effective agonist, wherein the induction or enhancement of the cellular response is indicative of the test compound being an effective agonist. In a preferred embodiment, the target cells have been engineered or treated to prevent expressing endogenous PRO-C-MG.2, PRO-C-MG.12, PRO-C-MG.45, PRO-C-MG.64 or PRO-C-MG.72 during the test period. The cellular response is preferably cell proliferation or tube formation.

In another embodiment, the invention provides a method for identifying a compound that inhibits the activity of a PRO-C-MG.2, PRO-C-MG.12, PRO-C-MG.45, PRO-C-MG.64 or PRO-C-MG.72 polypeptide comprising contacting a test compound with a PRO-C-MG.2, PRO-C-MG.12, PRO-C-MG.45, PRO-C-MG.64 or PRO-C-MG.72 polypeptide under conditions and for a time sufficient to allow the test compound and polypeptide to interact and determining whether the activity of the PRO-C-MG.2, PRO-C-MG.12, PRO-C-MG.45, PRO-C-MG.64 or PRO-C-MG.72 polypeptide is inhibited. In a specific preferred embodiment, either the test compound or the PRO-C-MG.2, PRO-C-MG.12, PRO-C-MG.45, PRO-C-MG.64 or PRO-C-MG.72 polypeptide is immobilized on a solid support. In another preferred aspect, the non-immobilized component carries a detectable label. In a preferred aspect, this method comprises the steps of: (a) contacting cells and a test compound to be screened in the presence of PRO-C-MG.2, PRO-C-MG.12, PRO-C-MG.45, PRO-C-MG.64 or PRO-C-MG.72 polypeptide under conditions suitable for the induction, stimulation, or dependence of a cellular response on a PRO-C-MG.2, PRO-C-MG.12, PRO-C-MG.45, PRO-C-MG.64 or PRO-C-MG.72 polypeptide; and (b) determining the induction, stimulation or dependence of the cellular response to determine if the test compound is an effective antagonist. In another preferred aspect, this process comprises the steps of: (a) contacting cells and a test compound to be screened in the presence of PRO-C-MG.2, PRO-C-MG.12, PRO-C-MG.45, PRO-C-MG.64 or PRO-C-MG.72 polypeptide under conditions suitable for the stimulation or dependence of cell proliferation or tube formation on a PRO-C-MG.2, PRO-C-MG.12, PRO-C-MG.45, PRO-C-MG.64 or PRO-C-MG.72 polypeptide; and (b) measuring the cell proliferation or tube formation to determine if the test compound is an effective antagonist.

In another embodiment, the invention provides a method for identifying a compound that inhibits the expression of a PRO-C-MG.2, PRO-C-MG.12, PRO-C-MG.45, PRO-C-MG.64 or PRO-C-MG.72 polypeptide in cells that normally expresses the polypeptide, wherein the method comprises contacting the cells with a test compound and determining whether the expression of the PRO-C-MG.2, PRO-C-MG.12, PRO-C-MG.45, PRO-C-MG.64 or PRO-C-MG.72 polypeptide is inhibited. In a preferred aspect, this method comprises the steps of: (a) contacting cells and a test compound to be screened under conditions suitable for allowing expression of the PRO-C-MG.2, PRO-C-MG.12, PRO-C-MG.45, PRO-C-MG.64 or PRO-C-MG.72 polypeptide; and (b) determining the inhibition of expression of said polypeptide.

In a still further embodiment, the invention provides a compound that inhibits the expression of a PRO-C-MG.2, PRO-C-MG.12, PRO-C-MG.45, PRO-C-MG.64 or PRO-C-MG.72 polypeptide, such as a compound that is identified by the methods set forth above.

Another aspect of the present invention is directed to an agonist or an antagonist of a PRO-C-MG.2, PRO-C-MG.12, PRO-C-MG.45, PRO-C-MG.64 or PRO-C-MG.72 polypeptide which can optionally be identified by the methods described above.

The invention also provides a microarray that comprises a polynucleotide sequence encoding PRO-C-MG.2, PRO-C-MG.12, PRO-C-MG.45, PRO-C-MG.64 or PRO-C-MG.72, optionally with a portion of the 5′ or 3′ untranslated sequence of the PRO-C-MG.2, PRO-C-MG.12, PRO-C-MG.45, PRO-C-MG.64 or PRO-C-MG.72 gene or mRNA.

In a still further embodiment, the invention concerns a composition of matter comprising a PRO-C-MG.2, PRO-C-MG.12, PRO-C-MG.45, PRO-C-MG.64 or PRO-C-MG.72 polypeptide, nucleic acid, or agonist or antagonist thereof as herein described, preferably an anti-PRO-C-MG.2, PRO-C-MG.12, PRO-C-MG.45, PRO-C-MG.64 or PRO-C-MG.72 antibody or a PRO-C-MG.2, PRO-C-MG.12, PRO-C-MG.45, PRO-C-MG.64 or PRO-C-MG.72 antigene molecule, in combination with a carrier. Optionally, the carrier is a pharmaceutically acceptable carrier.

Another embodiment of the present invention is directed to the use of a PRO-C-MG.2, PRO-C-MG.12, PRO-C-MG.45, PRO-C-MG.64 or PRO-C-MG.72 polypeptide or nucleic acid or an agonist or antagonist thereof as herein described. preferably an anti-PRO-C-MG.2, PRO-C-MG.12, PRO-C-MG.45, PRO-C-MG.64 or PRO-C-MG.72 antibody or a PRO-C-MG.2, PRO-C-MG.12, PRO-C-MG.45, PRO-C-MG.64 or PRO-C-MG.72 antigene molecule, for the preparation of a medicament useful in the treatment of a condition which is responsive to the PRO-C-MG.2, PRO-C-MG.12, PRO-C-MG.45, PRO-C-MG.64 or PRO-C-MG.72 polypeptide, nucleic acid, agonist or antagonist.

In another aspect, the present invention provides an article of manufacture comprising: (a) a composition of matter comprising a therapeutically effective dosage of a PRO-C-MG.2, PRO-C-MG.12, PRO-C-MG.45, PRO-C-MG.64 or PRO-C-MG.72 polypeptide or nucleic acid or an agonist or antagonist thereof as herein described, preferably an anti-PRO-C-MG.2, PRO-C-MG.12, PRO-C-MG.45, PRO-C-MG.64 or PRO-C-MG.72 antibody or a PRO-C-MG.2, PRO-C-MG.12, PRO-C-MG.45, PRO-C-MG.64 or PRO-C-MG.72 antigene molecule; (b) a container containing said composition; and optionally, (c) a label affixed to said container, or a package insert included in said pharmaceutical product referring to the use of the compound in the treatment of a cardiovascular, endothelial or angiogenic disorder.

In a still further aspect, the present invention provides a method for diagnosing a disease or susceptibility to a disease which is related to a mutation in a PRO-C-MG.2, PRO-C-MG.12, PRO-C-MG.45, PRO-C-MG.64 or PRO-C-MG.72 polypeptide-encoding nucleic acid sequence comprising: (a) isolating or amplifying a nucleic acid sequence encoding a PRO-C-MG.2, PRO-C-MG.12, PRO-C-MG.45, PRO-C-MG.64 or PRO-C-MG.72 polypeptide from a sample derived from a host; and (b) determining the presence or absence of said mutation in the PRO-C-MG.2, PRO-C-MG.12, PRO-C-MG.45, PRO-C-MG.64 or PRO-C-MG.72 polypeptide nucleic acid sequence, wherein the presence or absence of said mutation is indicative of the presence of said disease or susceptibility to said disease.

In a still further aspect, the invention provides a method of diagnosing a cardiovascular, endothelial or angiogenic disorder in a mammal which comprises analyzing the level of expression of a gene encoding a PRO-C-MG.2, PRO-C-MG.12, PRO-C-MG.45, PRO-C-MG.64 or PRO-C-MG.72 polypeptide (a) in a test sample of tissue cells obtained from said mammal, and (b) in a control sample of known normal tissue cells of the same cell type, wherein a higher or lower expression level in the test sample as compared to the control sample is indicative of the presence of a cardiovascular, endothelial or angiogenic disorder in said mammal. The expression of a gene encoding a PRO-C-MG.2, PRO-C-MG.12, PRO-C-MG.45, PRO-C-MG.64 or PRO-C-MG.72 polypeptide may optionally be accomplished by measuring the level of mRNA or polypeptide in the test sample as compared to the control sample.

In a still further aspect, the present invention provides a method of diagnosing a cardiovascular, endothelial or angiogenic disorder in a mammal which comprises detecting the presence or absence of a PRO-C-MG.2, PRO-C-MG.12, PRO-C-MG.45, PRO-C-MG.64 or PRO-C-MG.72 polypeptide in a test sample of tissue cells obtained from said mammal, wherein the presence or absence of said PRO-C-MG.2, PRO-C-MG.12, PRO-C-MG.45, PRO-C-MG.64 or PRO-C-MG.72 polypeptide in said test sample is indicative of the presence of a cardiovascular, endothelial or angiogenic disorder in said mammal.

In a still further embodiment, the invention provides a method of diagnosing a cardiovascular, endothelial or angiogenic disorder in a mammal comprising (a) contacting an anti-PRO-C-MG.2, PRO-C-MG.12, PRO-C-MG.45, PRO-C-MG.64 or PRO-C-MG.72 antibody with a test sample of tissue cells obtained from the mammal, and (b) detecting the formation of a complex between the antibody and the PRO-C-MG.2, PRO-C-MG.12, PRO-C-MG.45, PRO-C-MG.64 or PRO-C-MG.72 polypeptide in the test sample, wherein the formation of said complex is indicative of the presence of a cardiovascular, endothelial or angiogenic disorder in the mammal. The detection may be qualitative or quantitative, and may be performed in comparison with monitoring the complex formation in a control sample of known normal tissue cells of the same cell type. A larger or smaller quantity of complexes formed in the test sample indicates the presence of a cardiovascular, endothelial or angiogenic dysfunction in the mammal from which the test tissue cells were obtained. The antibody preferably carries a detectable label.

In another embodiment, the invention provides a method for determining the presence of a PRO-C-MG.2, PRO-C-MG.12, PRO-C-MG.45, PRO-C-MG.64 or PRO-C-MG.72 polypeptide in a sample comprising exposing a sample suspected of containing the PRO-C-MG.2, PRO-C-MG.12, PRO-C-MG.45, PRO-C-MG.64 or PRO-C-MG.72 polypeptide to an anti-PRO-C-MG.2, PRO-C-MG.12, PRO-C-MG.45, PRO-C-MG.64 or PRO-C-MG.72 antibody and determining binding of said antibody to a component of said sample.

In further aspects, the invention provides a cardiovascular, endothelial or angiogenic disorder diagnostic kit comprising an anti-PRO-C-MG.2, PRO-C-MG.12, PRO-C-MG.45, PRO-C-MG.64 or PRO-C-MG.72 antibody or nucleic acid, and a carrier, in suitable packaging. Preferably, such kit further comprises instructions for using said antibody or nucleic acid to detect the presence of the PRO-C-MG.2, PRO-C-MG.12, PRO-C-MG.45, PRO-C-MG.64 or PRO-C-MG.72 polypeptide or nucleic acid. Preferably, the carrier is a buffer, for example. Preferably, the cardiovascular, endothelial or angiogenic disorder is cancer.

In a further embodiment, the invention provides an article of manufacture, comprising: a container; a composition comprising a PRO-C-MG.2, PRO-C-MG.12, PRO-C-MG.45, PRO-C-MG.64 or PRO-C-MG.72 polypeptide contained within the container; and optionally, a label on the container, wherein the label on the container indicates that the composition can be used for treating cardiovascular, endothelial or angiogenic disorders.

In a further embodiment, the invention provides an article of manufacture, comprising: a container; a composition comprising a PRO-C-MG.2, PRO-C-MG.12, PRO-C-MG.45, PRO-C-MG.64 or PRO-C-MG.72 polypeptide agonist or antagonist contained within the container; and optionally a label on the container; wherein the label on the container indicates that the composition can be used for treating cardiovascular, endothelial or angiogenic disorders.

In a further embodiment, the invention provides an article of manufacture, comprising: a container; a composition comprising an anti-PRO-C-MG.2, PRO-C-MG.12, PRO-C-MG.45, PRO-C-MG.64 or PRO-C-MG.72 antibody or antigene compound contained within the container; and optionally a label on the container; wherein the label on the container indicates that the composition can be used for treating cardiovascular, endothelial or angiogenic disorders.

In yet another embodiment, the present invention provides a method for treating a cardiovascular, endothelial or angiogenic disorder in a mammal comprising administering to the mammal an effective amount of a PRO-C-MG.2, PRO-C-MG.12, PRO-C-MG.45, PRO-C-MG.64 or PRO-C-MG.72 polypeptide. Preferably, the disorder is cardiac hypertrophy, vascular trauma such as with wounds, bums, or surgery, or a type of cancer. In a further aspect, the mammal is further exposed to angioplasty or a drug that treats cardiovascular, endothelial or angiogenic disorders such as ACE inhibitors or chemotherapeutic agents if the cardiovascular, endothelial or angiogenic disorder is a type of cancer. Preferably, the mammal is human. Preferably it is one who is at risk of developing cardiac hypertrophy and more preferably has suffered myocardial infarction.

In another preferred embodiment, the cardiovascular, endothelial or angiogenic disorder is a cancer and the PRO-C-MG.2, PRO-C-MG.12, PRO-C-MG.45, PRO-C-MG.64 or PRO-C-MG.72 polypeptide is administered in combination with a chemotherapeutic agent, a growth inhibitory agent or a cytotoxic agent.

In a further embodiment, the invention concerns a method for treating a cardiovascular, endothelial or angiogenic disorder in a mammal comprising administering to the mammal an effective amount of an agonist of a PRO-C-MG.2, PRO-C-MG.12, PRO-C-MG.45, PRO-C-MG.64 or PRO-C-MG.72 polypeptide. Preferably, the cardiovascular, endothelial or angiogenic disorder is cardiac hypertrophy or vascular trauma. Also preferred is where the mammal is human, and where an effective amount of an angiogenic agent is administered in conjunction with the agonist.

In a further embodiment, the invention concerns a method for treating a cardiovascular, endothelial or angiogenic disorder in a mammal comprising administering to the mammal an effective amount of an antagonist of a PRO-C-MG.2, PRO-C-MG.12, PRO-C-MG.45, PRO-C-MG.64 or PRO-C-MG.72 polypeptide. Preferably, the cardiovascular, endothelial or angiogenic disorder is a cancer or age-related macular degeneration. Also preferred is where the mammal is human, and where an effective amount of an angiostatic agent is administered in conjunction with the antagonist.

In a further embodiment, the invention concerns a method for treating a cardiovascular, endothelial or angiogenic disorder in a mammal comprising administering to the mammal an effective amount of an anti-PRO-C-MG.2, PRO-C-MG.12, PRO-C-MG.45, PRO-C-MG.64 or PRO-C-MG.72 antibody or antigene compound. Preferably, the cardiovascular, endothelial or angiogenic disorder is cardiac hypertrophy, vascular trauma, a cancer, or age-related macular degeneration. Also preferred is where the mammal is human. Also preferred here is when an effective amount of an angiogenic or angiostatic agent is administered in conjunction with the antibody.

In still further embodiments, the invention provides a method for treating a cardiovascular, endothelial or angiogenic disorder in a mammal that suffers therefrom comprising administering to the mammal a nucleic acid molecule that is an antigene compound or that codes for either (a) a PRO-C-MG.2, PRO-C-MG.12, PRO-C-MG.45, PRO-C-MG.64 or PRO-C-MG.72 polypeptide (b) an agonist of a PRO-C-MG.2, PRO-C-MG.12, PRO-C-MG.45, PRO-C-MG.64 or PRO-C-MG.72 polypeptide or (c) an antagonist of a PRO-C-MG.2, PRO-C-MG.12, PRO-C-MG.45, PRO-C-MG.64 or PRO-C-MG.72 polypeptide, wherein said agonist or antagonist is preferably an anti-PRO-C-MG.2, PRO-C-MG.12, PRO-C-MG.45, PRO-C-MG.64 or PRO-C-MG.72 antibody. In a preferred embodiment the antigene compound is an antisense oligonucleotide, and more preferably a sense or antisense peptide nucleic acid. In a preferred embodiment, the mammal is human. In another preferred embodiment, the gene is administered via ex vivo gene therapy. In a further preferred embodiment, the gene is comprised within a vector, more preferably an adenoviral, adeno-associated viral, lentiviral, or retroviral vector.

In yet another aspect, the invention provides a recombinant retroviral particle comprising a retroviral vector consisting essentially of a promoter, a nucleic acid encoding (a) a PRO-C-MG.2, PRO-C-MG.12, PRO-C-MG.45, PRO-C-MG.64 or PRO-C-MG.72 polypeptide, (b) an agonist polypeptide of a PRO-C-MG.2, PRO-C-MG.12, PRO-C-MG.45, PRO-C-MG.64 or PRO-C-MG.72 polypeptide, or (c) an antagonist polypeptide of a PRO-C-MG.2, PRO-C-MG.12, PRO-C-MG.45, PRO-C-MG.64 or PRO-C-MG.72 polypeptide, and a signal sequence for cellular secretion of the polypeptide, wherein the retroviral vector is in association with retroviral structural proteins. Preferably, the signal sequence is from a mammal, such as from a native PRO-C-MG.2, PRO-C-MG.12, PRO-C-MG.45, PRO-C-MG.64 or PRO-C-MG.72 polypeptide.

In a still further embodiment, the invention supplies an ex vivo producer cell comprising a nucleic acid construct that expresses retroviral structural proteins and also comprises a retroviral vector consisting essentially of a promoter, nucleic acid encoding (a) a PRO-C-MG.2, PRO-C-MG.12, PRO-C-MG.45, PRO-C-MG.64 or PRO-C-MG.72 polypeptide, (b) an agonist polypeptide of a PRO-C-MG.2, PRO-C-MG.12, PRO-C-MG.45, PRO-C-MG.64 or PRO-C-MG.72 polypeptide or (c) an antagonist polypeptide of a PRO-C-MG.2, PRO-C-MG.12, PRO-C-MG.45, PRO-C-MG.64 or PRO-C-MG.72 polypeptide, and a signal sequence for cellular secretion of the polypeptide, wherein said producer cell packages the retroviral vector in association with the structural proteins to produce recombinant retroviral particles.

In yet another embodiment, the invention provides a method for inhibiting endothelial cell growth in a mammal comprising administering to the mammal (a) a PRO-C-MG.2, PRO-C-MG.12, PRO-C-MG.45, PRO-C-MG.64 or PRO-C-MG.72 polypeptide or (b) an antagonist of a PRO-C-MG.2, PRO-C-MG.12, PRO-C-MG.45, PRO-C-MG.64 or PRO-C-MG.72 polypeptide, where the antagonist is preferably a PRO-C-MG.2, PRO-C-MG.12, PRO-C-MG.45, PRO-C-MG.64 or PRO-C-MG.72 antigene compound or a small molecule, and wherein endothelial cell growth in said mammal is inhibited. Preferably, the mammal is human, and the endothelial cell growth is associated with a tumor.

In yet another embodiment, the invention provides a method for stimulating endothelial cell growth in a mammal comprising administering to the mammal (a) a PRO-C-MG.2, PRO-C-MG.12, PRO-C-MG.45, PRO-C-MG.64 or PRO-C-MG.72 polypeptide or (b) an agonist of a PRO-C-MG.2, PRO-C-MG.12, PRO-C-MG.45, PRO-C-MG.64 or PRO-C-MG.72 polypeptide, where preferably the agonist is a preferably a PRO-C-MG.2, PRO-C-MG.12, PRO-C-MG.45, PRO-C-MG.64 or PRO-C-MG.72 antigene compound, such as a PNA, or a small molecule, and wherein endothelial cell growth in the mammal is stimulated. Preferably, the mammal is human.

In yet another embodiment, the invention provides a method for inhibiting tube formation in a mammal comprising administering to the mammal (a) a PRO-C-MG.2, PRO-C-MG.12, PRO-C-MG.45, PRO-C-MG.64 or PRO-C-MG.72 polypeptide or (b) an antagonist of a PRO-C-MG.2, PRO-C-MG.12, PRO-C-MG.45, PRO-C-MG.64 or PRO-C-MG.72 polypeptide, wherein preferably the antagonist is a PRO-C-MG.2, PRO-C-MG.12, PRO-C-MG.45, PRO-C-MG.64 or PRO-C-MG.72 antigene compound or small molecule, and wherein tube formation in said mammal is inhibited.

In yet another embodiment, the invention provides a method for stimulating tube formation in a mammal comprising administering to the mammal (a) a PRO-C-MG.2, PRO-C-MG.12, PRO-C-MG.45, PRO-C-MG.64 or PRO-C-MG.72 polypeptide or (b) an agonist of a PRO-C-MG.2, PRO-C-MG.12, PRO-C-MG.45, PRO-C-MG.64 or PRO-C-MG.72 polypeptide, were the agonist is preferably a PRO-C-MG.2, PRO-C-MG.12, PRO-C-MG.45, PRO-C-MG.64 or PRO-C-MG.72 antigene compound or small molecule, and wherein tube formation in said mammal is stimulated.

In yet another embodiment, the invention provides a method for inhibiting angiogenesis induced by, enhanced by or dependent on a PRO-C-MG.2, PRO-C-MG.12, PRO-C-MG.45, PRO-C-MG.64 or PRO-C-MG.72 polypeptide in a mammal comprising administering to the mammal (a) a PRO-C-MG.2, PRO-C-MG.12, PRO-C-MG.45, PRO-C-MG.64 or PRO-C-MG.72 polypeptide or (b) an antagonist of a PRO-C-MG.2, PRO-C-MG.12, PRO-C-MG.45, PRO-C-MG.64 or PRO-C-MG.72 polypeptide, wherein preferably the antagonist is a PRO-C-MG.2, PRO-C-MG.12, PRO-C-MG.45, PRO-C-MG.64 or PRO-C-MG.72 antigene compound or small molecule, and wherein angiogenesis in the mammal is inhibited. Preferably, the mammal is a human, and more preferably the mammal has a tumor.

In yet another embodiment, the invention provides a method for stimulating angiogenesis induced by, enhanced by, or dependent on a PRO-C-MG.2, PRO-C-MG.12, PRO-C-MG.45, PRO-C-MG.64 or PRO-C-MG.72 polypeptide in a mammal comprising administering to the mammal (a) a PRO-C-MG.2, PRO-C-MG.12, PRO-C-MG.45, PRO-C-MG.64 or PRO-C-MG.72 polypeptide or (b) an agonist of a PRO-C-MG.2, PRO-C-MG.12, PRO-C-MG.45, PRO-C-MG.64 or PRO-C-MG.72 polypeptide, where the agonist is preferably a PRO-C-MG.2, PRO-C-MG.12, PRO-C-MG.45, PRO-C-MG.64 or PRO-C-MG.72 antigene compound or small molecule, and wherein angiogenesis in the mammal is stimulated. Preferably, the mammal is a human.

### BRIEF DESCRIPTION OF THE DRAWINGS

None.

### DETAILED DESCRIPTION OF THE PREFERRED EMBODIMENTS

1. Definitions

The terms “PRO-C-MG.2, PRO-C-MG.12, PRO-C-MG.45, PRO-C-MG.64 or PRO-C-MG.72 polypeptide”, “PRO-C-MG.2, PRO-C-MG.12, PRO-C-MG.45, PRO-C-MG.64 or PRO-C-MG.72 protein” and “PRO-C-MG.2, PRO-C-MG.12, PRO-C-MG.45, PRO-C-MG.64 or PRO-C-MG.72” when used herein encompass native sequence PRO-C-MG.2, PRO-C-MG.12, PRO-C-MG.45, PRO-C-MG.64 or PRO-C-MG.72 and PRO-C-MG.2, PRO-C-MG.12, PRO-C-MG.45, PRO-C-MG.64 or PRO-C-MG.72 polypeptide variants (which are further defined herein). The PRO-C-MG.2, PRO-C-MG.12, PRO-C-MG.45, PRO-C-MG.64 or PRO-C-MG.72 polypeptide can be isolated from a variety of sources, such as from human tissue types or from another source, or prepared by recombinant and/or synthetic methods.

A “native sequence PRO-C-MG.2, PRO-C-MG.12, PRO-C-MG.45, PRO-C-MG.64 or PRO-C-MG.72” comprises a polypeptide having the same amino acid sequence as a PRO-C-MG.2, PRO-C-MG.12, PRO-C-MG.45, PRO-C-MG.64 or PRO-C-MG.72 derived from nature. Such native sequence PRO-C-MG.2, PRO-C-MG.12, PRO-C-MG.45, PRO-C-MG.64 or PRO-C-MG.72 can be isolated from nature or can be produced by recombinant and/or synthetic means. The term “native sequence PRO-C-MG.2, PRO-C-MG.12, PRO-C-MG.45, PRO-C-MG.64 or PRO-C-MG.72” specifically encompasses naturally-occurring truncated or secreted forms (e.g., an extracellular domain sequence). Naturally-occurring variant forms (e.g., alternatively spliced forms) and naturally-occurring allelic variants of the PRO-C-MG.2, PRO-C-MG.12, PRO-C-MG.45, PRO-C-MG.64 or PRO-C-MG.72. In one embodiment of the invention, the native sequence PRO-C-MG.2, PRO-C-MG.12, PRO-C-MG.45, PRO-C-MG.64 or PRO-C-MG.72 is a mature or full-length native sequence PRO-C-MG.2, PRO-C-MG.12, PRO-C-MG.45, PRO-C-MG.64 or PRO-C-MG.72 comprising amino acids about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively. Also, while the PRO-C-MG.2, PRO-C-MG.12, PRO-C-MG.45, PRO-C-MG.64 or PRO-C-MG.72 polypeptide disclosed in SEQ ID NO:2 or SEQ ID NO:4, respectively, is shown to begin with the methionine residue designated herein as amino acid position 1, it is conceivable and possible that another methionine residue encoded by a start codon located either upstream or downstream from the codon of amino acid position 1 in SEQ ID NO:1 or SEQ ID NO:3 can be employed as the starting amino acid residue for the PRO-C-MG.2, PRO-C-MG.12, PRO-C-MG.45, PRO-C-MG.64 or PRO-C-MG.72 polypeptide.

“PRO-C-MG.2, PRO-C-MG.12, PRO-C-MG.45, PRO-C-MG.64 or PRO-C-MG.72 variant polypeptide” means an active PRO-C-MG.2, PRO-C-MG.12, PRO-C-MG.45, PRO-C-MG.64 or PRO-C-MG.72 polypeptide as defined herein having at least about 80% amino acid sequence identity with the amino acid sequence of (a) residues about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) another specifically derived fragment of the amino acid sequence shown in SEQ ID NO:2 or SEQ ID NO:4, respectively. Such PRO-C-MG.2, PRO-C-MG.12, PRO-C-MG.45, PRO-C-MG.64 or PRO-C-MG.72 variant polypeptides include, for instance, PRO-C-MG.2, PRO-C-MG.12, PRO-C-MG.45, PRO-C-MG.64 or PRO-C-MG.72 polypeptides wherein one or more amino acid residues are added, or deleted, at the N- and/or C-terminus, as well as within one or more internal domains, of the sequence of SEQ ID NO:2 or SEQ ID NO:4, respectively. Ordinarily, a PRO-C-MG.2, PRO-C-MG.12, PRO-C-MG.45, PRO-C-MG.64 or PRO-C-MG.72 variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with (a) residues about 1 to about 577 of SEQ ID NO:2, about 1 to about 474 of SEQ ID NO: 4, about 1 to about 506 of SEQ ID NO: 18. about 1 to about 344 of SEQ ID NO: 16, or about 1 to about 633 of SEQ ID NO: 14, respectively, or (b) another specifically derived fragment of the amino acid sequence shown in SEQ ID NO:2 or SEQ ID NO:4, respectively. PRO-C-MG.2, PRO-C-MG.12, PRO-C-MG.45, PRO-C-MG.64 or PRO-C-MG.72 variant polypeptides do not encompass the native PRO-C-MG.2, PRO-C-MG.12, PRO-C-MG.45, PRO-C-MG.64 or PRO-C-MG.72 polypeptide sequence. Ordinarily, PRO-C-MG.2, PRO-C-MG.12, PRO-C-MG.45, PRO-C-MG.64 or PRO-C-MG.72 variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

“Percent (%) amino acid sequence identity ” with respect to the PRO-C-MG2, PRO-C-MG.12, PRO-C-MG.45, PRO-C-MG.64 or PRO-C-MG.72 polypeptide sequences identified herein is defined as the percentage of amino acid residues in a candidate sequence that are identical with the amino acid residues in a PRO-C-MG.2, PRO-C-MG.12, PRO-C-MG.45, PRO-C-MG.64 or PRO-C-MG.72 sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2, wherein the complete source code for the ALIGN-2 program is provided in Table 1. The ALIGN-2 sequence comparison computer program was authored by Genentech, Inc. and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or can be compiled from the source code provided in Table 1. The ALIGN-2 program should be compiled for use on a UNIX operating system, preferably digital UNIX V4.0D. All sequence comparison parameters are set by the ALIGN-2 program and do not vary.

Hypothetical exemplifications are shown in Table 2, Comparisons 1 to 4, for determining % amino acid sequence identity (Table 2, Comparisons 1 and 2) and % nucleic acid sequence identity (Table 2, Comparisons 3 and 4) using the ALIGN-2 sequence comparison computer program, wherein “PRO” represents the amino acid sequence of a hypothetical PRO-C-MG.2, PRO-C-MG.12, PRO-C-MG.45, PRO-C-MG.64 or PRO-C-MG.72 polypeptide of interest, “Comparison Protein” represents the amino acid sequence of a polypeptide against which the “PRO” polypeptide of interest is being compared, “PRO-DNA” represents a hypothetical PRO-C-MG.2, PRO-C-MG.12, PRO-C-MG.45, PRO-C-MG.64 or PRO-C-MG.72-encoding nucleic acid sequence of interest, “Comparison DNA” represents the nucleotide sequence of a nucleic acid molecule against which the “PRO-DNA” nucleic acid molecule of interest is being compared, “X, “Y” and “Z” each represent different hypothetical amino acid residues and “N”, “L” and “V” each represent different hypothetical nucleotides.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations, Table 2 below presents comparisons 1 and 2 demonstrate how to calculate the % amino acid sequence identity of the amino acid sequence designated “Comparison Protein” to the amino acid sequence designated “PRO”.

TABLE 2

| Comparison 1 | PRO | XXXXXXXXXXXXXXX | (Length = 15 amino acids) |
|---|---|---|---|
| | Comparison Protein | XXXXXYYYYYYY | (Length = 12 amino acids) |
| | % amino acid sequence identity = (the | | |
| | number of identically matching amino acid residues between the two polypeptide | | |
| | sequences as determined by ALIGN-2) divided by (the total number of amino acid residues | | |
| | of the PRO polypeptide) = 5 divided by 15 = 33.3% | | |
| Comparison 2 | PRO | XXXXXXXXXX | (Length = 10 amino acids) |
| | Comparison Protein | XXXXXYYYYYYZZYZ | (Length = 15 amino acids) |
| | % amino acid sequence identity = (the | | |
| | number of identically matching amino acid residues between the two polypeptide | | |
| | sequences as determined by ALIGN-2) divided by (the total number of amino acid residues | | |
| | of the PRO polypeptide) = 5 divided by 10 = 50% | | |
| Comparison 3 | PRO-DNA | NNNNNNNNNNNNNN | (Length = 14 nucleotides) |
| | Comparison DNA | NNNNNNLLLLLLLLLL | (Length = 16 nucleotides) |
| | % nucleic acid sequence identity = (the | | |
| | number of identically matching nucleotides between the two nucleic acid sequences as | | |
| | determined by ALIGN-2) divided by (the total number of nucleotides of the PRO-DNA | | |
| | nucleic acid sequence) = 6 divided by 14 = 42.9% | | |
| Comparison 4 | PRO-DNA | NNNNNNNNNNNN | (Length = 12 nucleotides) |
| | Comparison DNA | NNNNLLLVV | (Length = 9 nucleotides) |

% nucleic acid sequence identity = (the
number of identically matching nucleotides between the two nucleic acid sequences as
determined by ALIGN-2) divided by (the total number of nucleotides of the PRO-DNA
nucleic acid sequence) = 4 divided by 12 = 33.3%

Unless specifically stated otherwise, all % amino acid sequence identity values used herein are obtained as described above using the ALIGN-2 sequence comparison computer program. However, % amino acid sequence identity can also be determined using the sequence comparison program NCBI-BLAST2 (Altschul et al., Nucleic Acids Res. 25:3389-3402 (1997)). The NCBI-BLAST2 sequence comparison program can be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“PRO-C-MG.2, PRO-C-MG.12, PRO-C-MG.45, PRO-C-MG.64 or PRO-C-MG.72 variant polynucleotide” or “PRO-C-MG.2, PRO-C-MG.12, PRO-C-MG.45, PRO-C-MG.64 or PRO-C-MG.72 variant nucleic acid sequence” means a nucleic acid molecule which encodes an active PRO-C-MG.2, PRO-C-MG.12, PRO-C-MG.45, PRO-C-MG.64 or PRO-C-MG.72 polypeptide as defined herein and which has at least about 80% nucleic acid sequence identity with either (a) a nucleic acid sequence which encodes residues about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) a nucleic acid sequence which encodes another specifically derived fragment of the amino acid sequence shown in SEQ ID NO:2 or SEQ ID NO:4, respectively. Ordinarily, a PRO-C-MG.2, PRO-C-MG.12, PRO-C-MG.45, PRO-C-MG.64 or PRO-C-MG.72 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes residues about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) a nucleic acid sequence which encodes another specifically derived fragment of the amino acid sequence shown in SEQ ID NO:2 or SEQ ID NO:4, respectively. PRO-C-MG.2, PRO-C-MG.12, PRO-C-MG.45, PRO-C-MG.64 or PRO-C-MG.72 polynucleotide variants do not encompass the native PRO-C-MG.2, PRO-C-MG.12, PRO-C-MG.45, PRO-C-MG.64 or PRO-C-MG.72 nucleotide sequence.

Ordinarily, PRO-C-MG.2, PRO-C-MG.12, PRO-C-MG.45, PRO-C-MG.64 or PRO-C-MG.72 variant polynucleotides are at least about 30 nucleotides in length, often at least about 60 nucleotides in length, more often at least about 90 nucleotides in length, more often at least about 120 nucleotides in length, more often at least about 150 nucleotides in length, more often at least about 180 nucleotides in length, more often at least about 210 nucleotides in length, more often at least about 240 nucleotides in length, more often at least about 270 nucleotides in length, more often at least about 300 nucleotides in length, more often at least about 450 nucleotides in length, more often at least about 600 nucleotides in length, more often at least about 900 nucleotides in length, or more.

“Percent (%) nucleic acid sequence identity” with respect to the PRO-C-MG.2, PRO-C-MG.12, PRO-C-MG.45, PRO-C-MG.64 or PRO-C-MG.72 polypeptide-encoding nucleic acid sequences identified herein is defined as the percentage of nucleotides in a candidate sequence that are identical with the nucleotides in a PRO-C-MG.2, PRO-C-MG.12, PRO-C-MG.45, PRO-C-MG.64 or PRO-C-MG.72 polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2, wherein the complete source code for the ALIGN-2 program is provided in Table 1. The ALIGN-2 sequence comparison computer program was authored by Genentech, Inc. and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or can be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Table 2, Comparisons 3 and 4, demonstrate how to calculate the % nucleic acid sequence identity of the nucleic acid sequence designated “Comparison DNA” to the nucleic acid sequence designated “PRO-DNA”.

Unless specifically stated otherwise, all % nucleic acid sequence identity values used herein are obtained as described above using the ALIGN-2 sequence comparison computer program. However, % nucleic acid sequence identity can also be determined using the sequence comparison program NCBI-BLAST2 (Altschul et al., Nucleic Acids Res. 25:3389-3402 (1997)). The NCBI-BLAST2 sequence comparison program can be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, PRO-C-MG.2, PRO-C-MG.12, PRO-C-MG.45, PRO-C-MG.64 or PRO-C-MG.72 variant polynucleotides are nucleic acid molecules that encode an active PRO-C-MG.2, PRO-C-MG.12, PRO-C-MG.45, PRO-C-MG.64 or PROC-MG.72 polypeptide and which are capable of hybridizing, preferably under stringent hybridization and wash conditions, to nucleotide sequences encoding the full-length PRO-C-MG.2, PRO-C-MG.12, PRO-C-MG.45, PRO-C-MG.64 or PRO-C-MG.72 polypeptide shown in SEQ ID NO:2 or SEQ ID NO:4, respectively. PRO-C-MG.2, PRO-C-MG.12, PRO-C-MG.45, PRO-C-MG.64 or PRO-C-MG.72 variant polypeptides can be those that are encoded by a PRO-C-MG.2, PRO-C-MG.12, PRO-C-MG.45, PRO-C-MG.64 or PRO-C-MG.72 variant polynucleotide.

The term “positives”, in the context of the amino acid sequence identity comparisons performed as described above, includes amino acid residues in the sequences compared that are not only identical, but also those that have similar properties. Amino acid residues that score a positive value to an amino acid residue of interest are those that are either identical to the amino acid residue of interest or are a preferred substitution (as defined in Table 3) of the amino acid residue of interest.

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows: 100 times the fraction X/Y, where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

“Isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and can include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO-C-MG.2, PRO-C-MG.12, PRO-C-MG.45, PRO-C-MG.64 or PRO-C-MG.72 natural environment will not be present. Ordinarily, however, isolated polypeptide will be prepared by at least one purification step.

An “isolated” nucleic acid molecule encoding a PRO-C-MG.2, PRO-C-MG.12, PRO-C-MG.45, PRO-C-MG.64 or PRO-C-MG.72 polypeptide is a nucleic acid molecule that is identified and separated from at least one contaminant nucleic acid molecule with which it is ordinarily associated in the natural source of the PRO-C-MG.2, PRO-C-MG.12, PRO-C-MG.45, PRO-C-MG.64 or PRO-C-MG.72-encoding nucleic acid. Preferably, the isolated nucleic is free of association with all components with which it is naturally associated. An isolated PRO-C-MG.2, PRO-C-MG.12, PRO-C-MG.45, PRO-C-MG.64 or PRO-C-MG.72-encoding nucleic acid molecule is other than in the form or setting in which it is found in nature. Isolated nucleic acid molecules therefore are distinguished from the PRO-C-MG.2, PRO-C-MG.12, PRO-C-MG.45, PRO-C-MG.64 or PRO-C-MG.72-encoding nucleic acid molecule as it exists in natural cells. However, an isolated nucleic acid molecule encoding a PRO-C-MG.2, PRO-C-MG.12, PRO-C-MG.45, PRO-C-MG.64 or PRO-C-MG.72 polypeptide includes PRO-C-MG.2, PRO-C-MG.12, PRO-C-MG.45, PRO-C-MG.64 or PRO-C-MG.72-encoding nucleic acid molecules contained in cells that ordinarily express PRO-C-MG.2, PRO-C-MG.12, PRO-C-MG.45, PRO-C-MG.64 or PRO-C-MG.72 where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PRO-C-MG.2, PRO-C-MG.12, PRO-C-MG.45, PRO-C-MG.64 or PRO-C-MG.72 monoclonal antibodies (including agonist, antagonoist, and neutralizing antibodies), anti-PRO-C-MG.2, PRO-C-MG.12, PRO-C-MG.45, PRO-C-MG.64 or PRO-C-MG.72 antibody compositions with polyepitopic specificity. Single chain anti-PRO-C-MG.2. PRO-C-MG.12, PRO-C-MG.45, PRO-C-MG.64 or PRO-C-MG.72 antibodies. and fragments of anti-PRO-C-MG.2, PRO-C-MG. 12, PRO-C-MG.45, PRO-C-MG.64 or PRO-C-MG.72 antibodies (see below). The term “monoclonal antibody” as used herein refers to an antibody obtained from a population of substantially homogeneous antibodies. i.e., the individual antibodies comprising the population are identical except for possible naturally-occurring mutations that can be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above.

An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO-C-MG.2, PRO-C-MG.12, PRO-C-MG.45, PRO-C-MG.64 or PRO-C-MG.72 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of PRO-C-MG.2, PRO-C-MG.12, PRO-C-MG.45, PRO-C-MG.64 or PRO-C-MG.72 which retain a biological and/or an immunological activity of native or naturally-occurring PRO-C-MG.2, PRO-C-MG.12, PRO-C-MG.45, PRO-C-MG.64 or PRO-C-MG.72, wherein “biological” activity refers to a biological function (either inhibitory or stimulatory), which includes enzymatic activity, caused by a native or naturally-occurring PRO-C-MG.2, PRO-C-MG.12, PRO-C-MG.45, PRO-C-MG.64 or PRO-C-MG.72 other than the ability to induce the production of an antibody against an antigenic epitope possessed by a native or naturally-occurring PRO-C-MG.2, PRO-C-MG.12, PRO-C-MG.45, PRO-C-MG.64 or PRO-C-MG.72 and an “immunological” activity refers to the ability to induce the production of an antibody against an antigenic epitope possessed by a native or naturally-occurring PRO-C-MG.2, PRO-C-MG.12, PRO-C-MG.45, PRO-C-MG.64 or PRO-C-MG.72.

The term “antagonist” is used in the broadest sense, and includes any molecule that partially or fully blocks, inhibits, or neutralizes a biological activity of a native PRO-C-MG.2, PRO-C-MG.12, PRO-C-MG.45, PRO-C-MG.64 or PRO-C-MG.72 polypeptide disclosed herein. In a similar manner, the term “agonist” is used in the broadest sense and includes any molecule that mimics a biological activity of a native PRO-C-MG.2, PRO-C-MG.12, PRO-C-MG.45, PRO-C-MG.64 or PRO-C-MG.72 polypeptide disclosed herein. Suitable agonist or antagonist molecules specifically include agonist or antagonist antibodies or antibody fragments, fragments or amino acid sequence variants of native PRO-C-MG.2, PRO-C-MG.12, PRO-C-MG.45, PRO-C-MG.64 or PRO-C-MG.72 polypeptides, peptides, antisense molecules, and small organic molecules. Methods for identifying agonists or antagonists of a PRO-C-MG.2, PRO-C-MG.12, PRO-C-MG.45, PRO-C-MG.64 or PRO-C-MG.72 polypeptide include contacting a PRO-C-MG.2, PRO-C-MG.12, PRO-C-MG.45, PRO-C-MG.64 or PRO-C-MG.72 polypeptide, mRNA or gene with a candidate agonist or antagonist molecule and measuring a detectable change in one or more biological activities normally associated with the PRO-C-MG.2, PRO-C-MG.12, PRO-C-MG.45, PRO-C-MG.64 or PRO-C-MG.72 polypeptide.

“Treatment” refers to both therapeutic treatment and prophylactic or preventative measures, wherein the object is to prevent or slow down (lessen) the targeted pathologic condition or disorder. More specifically, “treatment” is an intervention performed with the intention of preventing the development or altering the pathology of a cardiovascular, endothelial, neovascular or angiogenic disorder or condition. The concept of treatment is used in the broadest sense, and specifically includes the prevention (prophylaxis), moderation, reduction, and curing of the disorder or condition, at any stage. Accordingly, “treatment” refers to both therapeutic treatment and prophylactic or preventative measures, wherein the object is to prevent or slow down (lessen) said disorder or condition. The disorder may result from any cause, including idiopathic, cardiotrophic, or myotrophic causes, or ischemia or ischemic insults, such as myocardial infarction. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Microarray” refers to an array of distinct polynucleotides or oligonucleotides arranged on a substrate such as paper, nylon or other type of membrane, filter, gel, polymer, chip, glass slide, or any other suitable support, including solid supports. The polynucleotides or oligonucleotides (the backbone chemistry can be any available in the art) can be synthesized on a substrate or prepared before application to the substrate.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and can include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody so as to generate a “labeled” antibody. The label can be detectable by itself (e.g. radioisotope labels or fluorescent labels) or, in the case of an enzymatic label, can catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylaniides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO-C-MG.2, PRO-C-MG.12, PRO-C-MG.45, PRO-C-MG.64 or PRO-C-MG.72 polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The phrases “vascular or angiogenic disorder”, “vascular or angiogenic dysfunction” are used interchangeably and refer in part to systemic disorders that affect vessels, such as diabetes mellitus, as well as diseases of the vessels themselves, such as of the arteries, capillaries, veins, and/or lymphatics. This would include indications that stimulate angiogenesis, cardiovascularization, and/or neovascularization, and those that inhibit angiogenesis, cardiovascularization, and/or neovascularization.

“Hypertrophy”, as used herein, is defined as an increase in mass of an organ or structure independent of natural growth that does not involve tumor formation. Hypertrophy of an organ or tissue is due either to an increase in the mass of the individual cells (true hypertrophy), or to an increase in the number of cells making up the tissue (hyperplasia), or both. Certain organs, such as the heart, lose the ability to divide shortly after birth. Accordingly, “cardiac hypertrophy” is defined as an increase in mass of the heart, which, in adults, is characterized by an increase in myocyte cell size and contractile protein content without concomitant cell division. The character of the stress responsible for inciting the hypertrophy, (e.g., increased preload, increased afterload, loss of myocytes, as in myocardial infarction, or primary depression of contractility), appears to play a critical role in determining the nature of the response. The early stage of cardiac hypertrophy is usually characterized morphologically by increases in the size of myofibrils and mitochondria, as well as by enlargement of mitochondria and nuclei. At this stage, while muscle cells are larger than normal, cellular organization is largely preserved. At a more advanced stage of cardiac hypertrophy, there are preferential increases in the size or number of specific organelles, such as mitochondria, and new contractile elements are added in localized areas of the cells, in an irregular manner. Cells subjected to long-standing hypertrophy show more obvious disruptions in cellular organization, including markedly enlarged nuclei with highly lobulated membranes, which displace adjacent myofibrils and cause breakdown of normal Z-band registration. The phrase “cardiac hypertrophy” is used to include all stages of the progression of this condition, characterized by various degrees of structural damage of the heart muscle, regardless of the underlying cardiac disorder. Hence, the term also includes physiological conditions instrumental in the development of cardiac hypertrophy, such as elevated blood pressure, aortic stenosis, or myocardial infarction.

“Heart failure” refers to an abnormality of cardiac function where the heart does not pump blood at the rate needed for the requirements of metabolizing tissues. The heart failure can be caused by a number of factors, including ischemic, congenital, rheumatic, or idiopathic forms.

“Congestive heart failure” (CHF) is a progressive pathologic state where the heart is increasingly unable to supply adequate cardiac output (the volume of blood pumped by the heart over time) to deliver the oxygenated blood to peripheral tissues. As CHF progresses, structural and hemodynamic damages occur. While these damages have a variety of manifestations, one characteristic symptom is ventricular hypertrophy. CHF is a common end result of a number of various cardiac disorders.

“Myocardial infarction” generally results from atherosclerosis of the coronary arteries, often with superimposed coronary thrombosis. It may be divided into two major types: transmural infarcts, in which myocardial necrosis involves the full thickness of the ventricular wall, and subendocardial (nontransmural) infarcts, in which the necrosis involves the subendocardium, the intramural myocardium, or both, without extending all the way through the ventricular wall to the epicardium. Myocardial infarction is known to cause both a change in hemodynamic effects and an alteration in structure in the damaged and healthy zones of the heart. Thus, for example, myocardial infarction reduces the maximum cardiac output and the stroke volume of the heart. Also associated with myocardial infarction is a stimulation of the DNA synthesis occurring in the interstice as well as an increase in the formation of collagen in the areas of the heart not affected.

Supravalvular “aortic stenosis” is an inherited vascular disorder characterized by narrowing of the ascending aorta, but other arteries, including the pulmonary arteries, may also be affected. Untreated aortic stenosis may lead to increased intracardiac pressure resulting in myocardial hypertrophy and eventually heart failure and death. The pathogenesis of this disorder is not fully understood, but hypertrophy and possibly hyperplasia of medial smooth muscle are prominent features of this disorder. It has been reported that molecular variants of the elastin gene are involved in the development and pathogenesis of aortic stenosis. U.S. Pat. No. 5,650,282 issued Jul. 22, 1997.

“Valvular regurgitation” occurs as a result of heart diseases resulting in disorders of the cardiac valves. Various diseases, like rheumatic fever, can cause the shrinking or pulling apart of the valve orifice, while other diseases may result in endocarditis, an inflammation of the endocardium or lining membrane of the atrioventricular orifices and operation of the heart. Defects such as the narrowing of the valve stenosis or the defective closing of the valve result in an accumulation of blood in the heart cavity or regurgitation of blood past the valve. If uncorrected, prolonged valvular stenosis or insufficiency may result in cardiac hypertrophy and associated damage to the heart muscle, which may eventually necessitate valve replacement.

The treatment of all these, and other cardiovascular (endothelial-involved) and angiogenic disorders are encompassed by the present invention.

The terms “cancer”, “cancerous”, and “malignant” refer to or describe the physiological condition in mammals that is typically characterized by unregulated cell growth.

The term “neovascularization” refers to growth and development of blood vessels in tissue not normally containing them, or of blood vessels of a different kind than usual in tissue.

The term “cytotoxic agent” as used herein refers to a substance that inhibits or prevents the function of cells and/or causes destruction of cells. The term is intended to include radioactive isotopes (e.g., 1311, 1251, 90Y, and 186Re), chemotherapeutic agents, and toxins such as enzymatically active toxins of bacterial, fungal, plant, or animal origin, or fragments thereof.

A “chemotherapeutic agent” is a chemical compound useful in the treatment of cancer. Examples of chemotherapeutic agents include alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, or corticosteroids. Specific examples include Adriamycin, Doxorubicin, 5-Fluorouracil, Cytosine arabinoside (“Ara-C”), Cyclophosphamide, Thiotepa, Busulfan, Cytoxin, Taxol, Toxotere, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Canminomycin, Aminopterin, Dactinomycin, Mitomycins, Esperamicins (see U.S. Pat. No. 4,675,187), Melphalan, and other related nitrogen mustards. Also included in this definition are hormonal agents that act to regulate or inhibit hormone action on tumors, such as tamoxifen and onapristone.

A “growth-inhibitory agent” when used herein refers to a compound or composition that inhibits growth of a cell, such as an Wnt-overexpressing cancer cell, either in vitro or in vivo, and includes and is used interchangeably herein with angiostatic agents. Thus, the growth-inhibitory agent is one which significantly reduces the percentage of malignant cells in S phase, for example. Examples of growth-inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. Additional examples include tumor necrosis factor (TNF), an antibody capable of inhibiting or neutralizing the angiogenic activity of acidic or basic FGF or hepatocyte growth factor (HGF), an antibody capable of inhibiting or neutralizing the coagulant activities of tissue factor, protein C, or protein S (see, WO 91/01753, published 21 Feb. 1991), or an antibody capable of binding to HER2 receptor (WO 89/06692), such as the 4D5 antibody (and functional equivalents thereof) (e.g., WO 92/22653).

A “cardiovascular agent” refers generically to any drug that acts in treating cardiovascular disorders. Examples of cardiovascular agents are those that promote vascular homeostasis by modulating blood pressure, heart rate, heart contractility, and endothelial and smooth muscle biology, all of which factors have a role in cardiovascular disease. Specific examples of these include angiotensin-II receptor antagonists; endothelin receptor antagonists such as, for example, BOSENTAN™ and MOXONODIN™; interferon-gamma (IFN-γ); des-aspartate-angiotensin I; thrombolytic agents, e.g., streptokinase, urokinase, t-PA, and a t-PA variant specifically designed to have longer half-life and very high fibrin specificity, TNK t-PA (a T103N, N117Q, KHRR(296-299)AAAA t-PA variant, Keyt et al, Proc. Natl. Acad. Sci. USA 91, 3670-3674 (1994)); inotropic or hypertensive agents such as digoxigenin and β-adrenergic receptor blocking agents, e.g., propranolol, timolol, tertalolol, carteolol, nadolol, betaxolol, penbutolol, acetobutolol, atenolol, metoprolol, and carvedilol; angiotensin converting enzyme (ACE) inhibitors, e.g., quinapril, captopril, enalapril, ramipril, benazepril, fosinopril, and lisinopril; diuretics, e.g., chlorothiazide, hydrochlorothiazide, hydroflumethazide, methylchlothiazide, benzthiazide, dichlorpheiainide, acetazolamide, and indapamide; and calcium channel blockers, e.g., diltiazem, nifedipine, verapamil, nicardipine.

“Angiogenic agents” and “endothelial agents” are active agents that promote angiogenesis and/or endothelial cell growth, or, if applicable, vasculogenesis. This would include factors that accelerate wound healing, such as growth hormone, insulin-like growth factor-1 (IGF-1), VEGF, VIGF, PDGF, epidermal growth factor (EGF), CTGF and members of its family, FGF, and TGF-α and TGF-β.

“Angiostatic agents” are active agents that inhibit angiogenesis or vasculogenesis or otherwise inhibit or prevent growth of cancer cells. Examples include antibodies or other antagonists to angiogenic agents as defined above, such as antibodies to VEGF. They additionally include cytotherapeutic agents such as cytotoxic agents, chemotherapeutic agents, growth-inhibitory agents, apoptotic agents, and other agents to treat cancer, such as anti-HER-2, anti-CD20, and other bioactive and organic chemical agent.

“Endothelial cell” means the cells of endothelial tissue, which includes the membranes lining serous cavities, heart, blood and lymph vessels.

In a pharmacological sense, in the context of the present invention, a “therapeutically effective amount” of an active agent such as a PRO-C-MG.2, PRO-C-MG.12, PRO-C-MG.45, PRO-C-MG.64 or PROC-MG.72 polypeptide or agonist or antagonist thereto or an anti-PRO-C-MG.2, PRO-C-MG.12, PRO-C-MG.45, PRO-C-MG.64 or PRO-C-MG.72 antibody, refers to an amount effective in the treatment of a cardiovascular, endothelial or angiogenic disorder in a mammal and can be determined empirically. An effective amount will either prevent, lessen the worsening of, alleviate, or cure the treated condition., or stimulate, enhance, reduce or inhibit the cellular response, biological activity, or stated purpose.

As used herein, an “effective amount” of an active agent such as a PRO-C-MG.2, PRO-C-MG.12, PRO-C-MG.45, PRO-C-MG.64 or PRO-C-MG.72 polypeptide or agonist or antagonist thereto or an anti-PRO-C-MG.2, PRO-C-MG.12, PRO-C-MG.45, PRO-C-MG.64 or PRO-C-MG.72 antibody, refers to an amount effective for carrying out a stated purpose, wherein such amounts may be determined empirically for the desired effect. An effective amount can stimulate, enhance, reduce or inhibit the cellular response, biological activity, or other stated purpose.

II. Compositions and Methods of the Invention

A. Full-Length PRO-C-MG.2, PRO-C-MG.12, PRO-C-MG.45, PRO-C-MG.64 or PRO-C-MG.72 Polypeptide

The present invention provides newly identified and isolated nucleotide sequences encoding polypeptides referred to in the present application as PRO-C-MG.2, PRO-C-MG.12, PRO-C-MG.45, PRO-C-MG.64 or PRO-C-MG.72. In particular, cDNA encoding a PRO-C-MG.2, PRO-C-MG.12, PRO-C-MG.45, PRO-C-MG.64 or PRO-C-MG.72 polypeptide has been identified and isolated, as disclosed in further detail in the Examples below. For sake of simplicity, in the present specification the protein encoded by DNA-C-MG.2-1776, DNA-C-MG.12-1776, DNA-C-MG.45-1776, DNA-C-MG.64-1776 or DNA-C-MG.72-1776 as well as all further native homologues and variants included in the foregoing definition of PRO-C-MG.2, PRO-C-MG.12, PRO-C-MG.45, PRO-C-MG.64 or PRO-C-MG.72, will be referred to as “PRO-C-MG.2, PRO-C-MG.12, PRO-C-MG.45, PRO-C-MG.64 or PRO-C-MG.72”, regardless of their origin or mode of preparation.

As disclosed in the Examples below, a cDNA clone designated herein as DNA-C-MG.2-1776, DNA-C-MG.12-1776, DNA-C-MG.45-1776, DNA-C-MG.64-1776 or DNA-C-MG.72-1776 has been deposited with the ATCC. The actual nucleotide sequence of the clone can readily be determined by the skilled artisan by sequencing of the deposited clone using routine methods in the art. The predicted amino acid sequence can be determined from the nucleotide sequence using routine skill. For the PRO-C-MG.2, PRO-C-MG.12, PRO-C-MG.45, PRO-C-MG.64 or PRO-C-MG.72 polypeptide and encoding nucleic acid described herein, Applicants have identified what is believed to be the reading frame best identifiable with the sequence information available at the time.

SEQ ID NO:1 shows a cDNA containing a nucleotide sequence (nucleotides 1-2891) encoding native sequence PRO-C-MG.2, wherein the nucleotide sequence (SEQ ID NO:1) is a clone designated herein as “DNA-C-MG.2-1776.” SEQ ID NO:2 shows the amino acid sequence (SEQ ID NO:2) of a native sequence PRO-C-MG.2 polypeptide as derived from the coding sequence of SEQ ID NO:1.

SEQ ID NO:3 shows a cDNA containing a nucleotide sequence (nucleotides 1-2119) encoding native sequence PRO-C-MG.12, wherein the nucleotide sequence (SEQ ID NO:3) is a clone designated herein as “DNA-C-MG.12-1776.” SEQ ID NO:4 shows the amino acid sequence (SEQ ID NO:4) of a native sequence PRO-C-MG.12 polypeptide as derived from the coding sequence of SEQ ID NO:3.

B. PRO-C-MG.2, PRO-C-MG.12, PRO-C-MG.45, PRO-C-MG.64 or PRO-C-MG.72 Variants

In addition to the full-length native sequence PRO-C-MG.2, PRO-C-MG.12, PRO-C-MG.45, PRO-C-MG.64 or PRO-C-MG.72 polypeptides described herein, it is contemplated that PRO-C-MG.2, PRO-C-MG.12, PRO-C-MG.45, PRO-C-MG.64 or PRO-C-MG.72 variants can be prepared. PRO-C-MG.2, PRO-C-MG.12, PRO-C-MG.45, PRO-C-MG.64 or PRO-C-MG.72 variants can be prepared by introducing appropriate nucleotide changes into the PRO-C-MG.2, PRO-C-MG.12, PRO-C-MG.45, PRO-C-MG.64 or PRO-C-MG.72 DNA, and/or by synthesis of the desired PRO-C-MG.2, PRO-C-MG.12, PRO-C-MG.45, PRO-C-MG.64 or PRO-C-MG.72 polypeptide. Those skilled in the art will appreciate that amino acid changes can alter post-translational processes of the PRO-C-MG.2, PRO-C-MG.12, PRO-C-MG.45, PRO-C-MG.64 or PRO-C-MG.72, such as changing the number or position of glycosylation sites or altering the membrane anchoring characteristics.

Variations in the native full-length sequence PRO-C-MG.2, PRO-C-MG.12, PRO-C-MG.45, PRO-C-MG.64 or PRO-C-MG.72 or in various domains of the PRO-C-MG.2, PRO-C-MG.12, PRO-C-MG.45, PRO-C-MG.64 or PRO-C-MG.72 described herein, can be made, for example, using any of the techniques and guidelines for conservative and non-conservative mutations set forth, for instance, in U.S. Pat. No. 5,364,934. Variations can be a substitution, deletion or insertion of one or more codons encoding the PRO-C-MG.2, PRO-C-MG.12, PRO-C-MG.45, PRO-C-MG.64 or PRO-C-MG.72 that results in a change in the amino acid sequence of the PRO-C-MG.2, PRO-C-MG.12, PRO-C-MG.45, PRO-C-MG.64 or PRO-C-MG.72 as compared with the native sequence PRO-C-MG.2, PRO-C-MG.12, PRO-C-MG.45, PRO-C-MG.64 or PRO-C-MG.72. Optionally the variation is by substitution of at least one amino acid with any other amino acid in one or more of the domains of the PRO-C-MG.2, PRO-C-MG.12, PRO-C-MG.45, PRO-C-MG.64 or PRO-C-MG.72. Guidance in determining which amino acid residue can be inserted, substituted or deleted without adversely affecting the desired activity can be found by comparing the sequence of the PRO-C-MG.2, PRO-C-MG.12, PRO-C-MG.45, PRO-C-MG.64 or PRO-C-MG.72 with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of about 1 to 5 amino acids. The variation allowed can be determined by systematically making insertions, deletions or substitutions of amino acids in the sequence and testing the resulting variants for activity exhibited by the full-length or mature native sequence.

PRO-C-MG.2, PRO-C-MG.12, PRO-C-MG.45, PRO-C-MG.64 or PRO-C-MG.72 polypeptide fragments are provided herein. Such fragments can be truncated at the N-terminus or C-terminus, or can lack internal residues, for example, when compared with a full length native protein. Certain fragments lack amino acid residues that are not essential for a desired biological activity of the PRO-C-MG2, PRO-C-MG.12, PRO-C-MG.45, PRO-C-MG.64 or PRO-C-MG.72 polypeptide.

PRO-C-MG2, PRO-C-MG.12, PRO-C-MG.45, PRO-C-MG.64 or PRO-C-MG.72 fragments can be prepared by any of a number of conventional techniques. Desired peptide fragments can be chemically synthesized. An alternative approach involves generating PRO-C-MG.2, PRO-C-MG.12, PRO-C-MG.45, PRO-C-MG.64 or PRO-C-MG.72 fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR Preferably, PRO-C-MG.2, PRO-C-MG.12, PRO-C-MG.45, PRO-C-MG.64 or PRO-C-MG.72 polypeptide fragments share at least one biological and/or immunological activity with the native PRO-C-MG.2, PRO-C-MG.12, PRO-C-MG.45, PRO-C-MG.64 or PRO-C-MG.72 polypeptide shown in SEQ ID NO:2 or SEQ ID NO:4, respectively.

In particular embodiments, conservative substitutions of interest are shown in Table 3 under the heading of preferred substitutions. If such substitutions result in a change in biological activity, then more substantial changes, denominated exemplary substitutions in Table 3, or as further described below in reference to amino acid classes, are introduced and the products screened.

TABLE 3

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr leu | |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the PRO-C-MG.2, PRO-C-MG.12, PRO-C-MG.45, PRO-C-MG.64 or PRO-C-MG.72 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic: trp, tyr, phe.

Non-conservative substitutions will entail exchanging a member of one of these classes for another class. Such substituted residues also can be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the PRO-C-MG.2, PRO-C-MG.12, PRO-C-MG.45, PRO-C-MG.64 or PRO-C-MG.72 variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

C. Modifications of PRO-C-MG.2, PRO-C-MG.12, PRO-C-MG.45, PRO-C-MG.64 or PRO-C-MG.72

Covalent modifications of PRO-C-MG.2, PRO-C-MG.12, PRO-C-MG.45, PRO-C-MG.64 or PRO-C-MG.72 are included within the scope of this invention. One type of covalent modification includes reacting targeted amilo acid residues of a PRO-C-MG.2, PRO-C-MG.12, PRO-C-MG.45, PRO-C-MG.64 or PRO-C-MG.72 polypeptide with an organic derivatizing agent that is capable of reacting with selected side chains or the N- or C-terminal residues of the PRO-C-MG.2, PRO-C-MG.12, PRO-C-MG.45, PRO-C-MG.64 or PRO-C-MG.72. Derivatization with bifunctional agents is useful, for instance, for crosslinking PRO-C-MG.2, PRO-C-MG.12, PRO-C-MG.45, PRO-C-MG.64 or PRO-C-MG.72 to a water-insoluble support matrix or surface for use in the method for purifying anti-PRO-C-MG.2, PRO-C-MG.12, PRO-C-MG.45, PRO-C-MG.64 or PRO-C-MG.72 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioim idate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO-C-MG.2, PRO-C-MG.12, PRO-C-MG.45, PRO-C-MG.64 or PRO-C-MG.72 polypeptide included within the scope of this invention comprises altering the native glycosylation pattern of the polypeptide. “Altering the native glycosylation pattern” is intended for purposes herein to mean deleting one or more carbohydrate moieties found in native sequence PRO-C-MG.2, PRO-C-MG.12, PRO-C-MG.45, PRO-C-MG.64 or PRO-C-MG.72 (either by removing the underlying glycosylation site or by deleting the glycosylation by chemical and/or enzymatic means), and/or adding one or more glycosylation sites that are not present in the native sequence PRO-C-MG.2, PRO-C-MG.12, PRO-C-MG.45, PRO-C-MG.64 or PRO-C-MG.72. In addition, the phrase includes qualitative changes in the glycosylation of the native proteins, involving a change in the nature and proportions of the various carbohydrate moieties present.

Addition of glycosylation sites to the PRO-C-MG.2, PRO-C-MG.12, PRO-C-MG.45, PRO-C-MG.64 or PRO-C-MG.72 polypeptide can be accomplished by altering the amino acid sequence. The alteration can be made, for example, by the addition of, or substitution by, one or more serine or threonine residues to the native sequence PRO-C-MG.2, PRO-C-MG.12, PRO-C-MG.45, PRO-C-MG.64 or PRO-C-MG.72 (for O-linked glycosylation sites). The PRO-C-MG.2, PRO-C-MG.12, PRO-C-MG.45, PRO-C-MG.64 or PRO-C-MG.72 amino acid sequence can optionally be altered through changes at the DNA level, particularly by mutating the DNA encoding the PRO-C-MG.2, PRO-C-MG.12, PRO-C-MG.45, PRO-C-MG.64 or PRO-C-MG.72 polypeptide at preselected bases such that codons are generated that will translate into the desired amino acids.

Another means of increasing the number of carbohydrate moieties on the PRO-C-MG.2, PRO-C-MG.12, PRO-C-MG.45, PRO-C-MG.64 or PRO-C-MG.72 polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO-C-MG.2, PRO-C-MG.12, PRO-C-MG.45, PRO-C-MG.64 or PRO-C-MG.72 polypeptide can be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO-C-MG.2, PRO-C-MG.12, PRO-C-MG.45, PRO-C-MG.64 or PRO-C-MG.72 comprises linking the PRO-C-MG.2, PRO-C-MG.12, PRO-C-MG.45, PRO-C-MG.64 or PRO-C-MG.72 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The PRO-C-MG.2, PRO-C-MG.12, PRO-C-MG.45, PRO-C-MG.64 or PRO-C-MG.72 of the present invention can also be modified in a way to form a chimeric molecule comprising PRO-C-MG.2, PRO-C-MG.12, PRO-C-MG.45, PRO-C-MG.64 or PRO-C-MG.72 fused to another, heterologous polypeptide or amino acid sequence.

In one embodiment, such a chimeric molecule comprises a fusion of the PRO-C-MG.2, PRO-C-MG.12, PRO-C-MG.45, PRO-C-MG.64 or PRO-C-MG.72 with a tag polypeptide which provides an epitope to which an anti-tag antibody can selectively bind. The epitope tag is generally placed at the amino- or carboxyl-terminus of the PRO-C-MG.2, PRO-C-MG.12, PRO-C-MG.45, PRO-C-MG.64 or PRO-C-MG.72. The presence of such epitope-tagged forms of the PRO-C-MG.2, PRO-C-MG.12, PRO-C-MG.45, PRO-C-MG.64 or PRO-C-MG.72 can be detected using an antibody against the tag polypeptide. Also, provision of the epitope tag enables the PRO-C-MG.2, PRO-C-MG.12, PRO-C-MG.45, PRO-C-MG.64 or PRO-C-MG.72 to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule can comprise a fusion of the PRO-C-MG.2, PRO-C-MG.12, PRO-C-MG.45, PRO-C-MG.64 or PRO-C-MG.72 with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) fonn of a PRO-C-MG.2, PRO-C-MG.12, PRO-C-MG.45, PRO-C-MG.64 or PRO-C-MG.72 polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

In another embodiment, the chimeric molecule includes a fusion of a PRO-C-MG.2, PRO-C-MG.12, PRO-C-MG.45, PRO-C-MG.64 or PRO-C-MG.72 with a signal peptide to allow or enhance secretion of the PRO-C-MG.2, PRO-C-MG.12, PRO-C-MG.45, PRO-C-MG.64 or PRO-C-MG.72 peptide or even to change its localization within the host cell. The signal sequence is generally placed at the amino- or carboxyl-terminus of the PRO-C-MG.2, PRO-C-MG.12, PRO-C-MG.45, PRO-C-MG.64 or PRO-C-MG.72, more usually the N-terminus when secretion or membrane localization is desired. Such fusions are typically intermediate products, since the signal peptide is usually specifically cleaved by enzymes of the host cell. Provision of a signal peptide enables the PRO-C-MG.2, PRO-C-MG.12, PRO-C-MG.45, PRO-C-MG.64 or PRO-C-MG.72 to be readily purified following its secretion to the culture medium. Various signal polypeptides, which allow secretion or targeting to compartments within the cell, are well known in the art and are available for use with numerous host cells, including yeast and mammalian cells.

D. Preparation of PRO-C-MG.2, PRO-C-MG.12, PRO-C-MG.45, PRO-C-MG.64 or PRO-C-MG.72

The description below relates primarily to production of PRO-C-MG.2, PRO-C-MG.12, PRO-C-MG.45, PRO-C-MG.64 or PRO-C-MG.72 by culturing cells transformed or transfected with a vector containing PRO-C-MG.2, PRO-C-MG.12, PRO-C-MG.45, PRO-C-MG.64 or PRO-C-MG.72 nucleic acid. It is, of course, contemplated that alternative methods, which are well known in the art, can be employed to prepare PRO-C-MG.2, PRO-C-MG.12, PRO-C-MG.45, PRO-C-MG.64 or PRO-C-MG.72. For instance, the PRO-C-MG.2, PRO-C-MG.12, PRO-C-MG.45, PRO-C-MG.64 or PRO-C-MG.72 sequence, or portions thereof, can be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis can be performed using manual techniques or by automation. Automated synthesis can be accomplished, for instance, using an Applied Biosystems Peptide Synthesizer (Foster City, Calif.) using manufacturer's instructions. Various portions of the PRO-C-MG.2, PRO-C-MG.12, PRO-C-MG.45, PRO-C-MG.64 or PRO-C-MG.72 can be chemically synthesized separately and combined using chemical or enzymatic methods to produce the full-length PRO-C-MG.2, PRO-C-MG.12, PRO-C-MG.45, PRO-C-MG.64 or PRO-C-MG.72.

1. Isolation of DNA Encoding PRO-C-MG.2, PRO-C-MG.12, PRO-C-MG.45, PRO-C-MG.64 or PRO-C-MG.72

DNA encoding PRO-C-MG.2, PRO-C-MG.12, PRO-C-MG.45, PRO-C-MG.64 or PRO-C-MG.72 can be obtained from a cDNA library prepared from tissue believed to possess the PRO-C-MG.2, PRO-C-MG.12, PRO-C-MG.45, PRO-C-MG.64 or PRO-C-MG.72 mRNA and to express it at a detectable level. Accordingly, human PRO-C-MG.2, PRO-C-MG.12, PRO-C-MG.45, PRO-C-MG.64 or PRO-C-MG.72 DNA can be conveniently obtained from a cDNA library prepared from human tissue, such as described in the Examples. The PRO-C-MG.2, PRO-C-MG.12, PRO-C-MG.45, PRO-C-MG.64 or PRO-C-MG.72-encoding gene can also be obtained from a genomic library or by known synthetic procedures (e.g. automated nucleic acid synthesis).

Libraries can be screened with probes (such as antibodies to the PRO-C-MG.2, PRO-C-MG.12, PRO-C-MG.45, PRO-C-MG.64 or PRO-C-MG.72 or oligonucleotides of at least about 20-80 bases) designed to identify the gene of interest or the protein encoded by it. Screening the cDNA or genomic library with the selected probe can be conducted using standard procedures, such as described in Sambrook et al., Molecular Cloning: A Laboratory Manual (New York: Cold Spring Harbor Laboratory Press, 1989). An alternative means to isolate the gene encoding PRO-C-MG.2, PRO-C-MG.12, PRO-C-MG.45, PRO-C-MG.64 or PRO-C-MG.72 is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence can be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO-C-MG.2, PRO-C-MG.12, PRO-C-MG.45, PRO-C-MG.64 or PRO-C-MG.72 production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyomithine, can also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 can be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT karl; E. coli W3110 strain 37D6, which has the complete genotype tonA, ptr3 phoA E15 (argF-lac)169 degP ompT rbs7 ilvG karl; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO-C-MG.2, PRO-C-MG.12, PRO-C-MG.45, PRO-C-MG.64 or PRO-C-MG.72-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technolopy, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K bulgaricus (ATCC 16,045), K. wickeramii (ATCC 24,178), K. waltii (ATCC 56,500), K. drosophilarum (ATCC 36,906; Van den Berg et al., Bio/Technolgy, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilbum et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts can be found in C. Anthony, The Biochemistry of Methylotrophs, 269 (1982).

Suitable host cells for the expression of glycosylated PRO-C-MG.2, PRO-C-MG.12, PRO-C-MG.45, PRO-C-MG.64 or PRO-C-MG.72 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding PRO-C-MG.2, PRO-C-MG.12, PRO-C-MG.45, PRO-C-MG.64 or PRO-C-MG.72 can be inserted into a replicable vector for cloning (amplification of the DNA) or for expression. Various vectors are publicly available. The vector can, for example, be in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO-C-MG.2, PRO-C-MG.12, PRO-C-MG.45, PRO-C-MG.64 or PRO-C-MG.72 can be produced recombinantly not only directly, but also as a fusion polypeptide with a heterologous polypeptide, which can be a signal sequence or other polypeptide having a specific cleavage site at the N-terminus of the mature protein or polypeptide. In general, the signal sequence can be a component of the vector, or it can be a part of the PRO-C-MG.2, PRO-C-MG.12, PRO-C-MG.45, PRO-C-MG.64 or PRO-C-MG.72-encoding DNA that is inserted into the vector. The signal sequence can be a prokaryotic signal sequence selected, for example, from the group of the alkaline phosphatase, penicillinase, lpp, or heat-stable enterotoxin II leaders. For yeast secretion the signal sequence can be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences can be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. An example of suitable selectable markers for mammalian cells are those that enable the identification of cells competent to take up the PRO-C-MG.2, PRO-C-MG.12, PRO-C-MG.45, PRO-C-MG.64 or PRO-C-MG.72-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PRO-C-MG.2, PRO-C-MG.12, PRO-C-MG.45, PRO-C-MG.64 or PRO-C-MG.72-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgamo (S.D.) sequence operably linked to the DNA encoding PRO-C-MG.2, PRO-C-MG.12, PRO-C-MG.45, PRO-C-MG.64 or PRO-C-MG.72.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-pilosphate dehydrogenase, hexokinase, pyruvate decarboxylase. phosphofructokinase, glucose-6-phosphate isomerase, 3-phosphoglycerate mutase, pyruvate kinase. triosephosphate isomerase, phosphogucose isomerase. and glucokinase.

Other yeast promoters, which are inducible promoters having the additional advantage of transcription controlled by growth conditions. are the promoter regions for alcohol dehydrogenase 2. isocnytochrome C. acid phosphatase. degradative enzymes associated with nitrogen metabolism, metallothioncin. glyceraldehyde-3-phosphate dehydrogenase, and enzymes responsible for maltose and galactose utilization. Suitable vectors and promoters for use in yeast expression are further described in EP 73,657.

PRO-C-MG.2, PRO-C-MG.12, PRO-C-MG.45, PRO-C-MG.64 or PRO-C-MG.72 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO-C-MG.2, PRO-C-MG.12, PRO-C-MG.45, PRO-C-MG.64 or PRO-C-MG.72 by higher eukaryotes can be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer can be spliced into the vector at a position 5′ or 3′ to the PRO-C-MG.2, PRO-C-MG.12, PRO-C-MG.45, PRO-C-MG.64 or PRO-C-MG.72 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO-C-MG.2, PRO-C-MG.12, PRO-C-MG.45, PRO-C-MG.64 or PRO-C-MG.72.

Still other methods, vectors, and host cells suitable for adaptation to the synthesis of PRO-C-MG.2, PRO-C-MG.12, PRO-C-MG.45, PRO-C-MG.64 or PRO-C-MG.72 in recombinant vertebrate cell culture are described in Gething et al., Nature, 293:620-625 (198 1); Mantei et al., Nature, 281:40-46 (1979); EP 117,060; and EP 117,058.

4. Detecting Gene Amplification/Expression

Gene amplification and/or expression can be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of cells or tissue sections and assay of cell culture or body fluids, to quantitate directly the expression of gene product. Antibodies useful for immunohistochemical staining and/or assay of sample fluids can be either monoclonal or polyclonal, and can be prepared in any mammal. Conveniently, the antibodies can be prepared against a native sequence PRO-C-MG.2, PRO-C-MG.12, PRO-C-MG.45, PRO-C-MG.64 or PRO-C-MG.72 polypeptide or against a synthetic peptide based on the DNA sequences provided herein or against exogenous sequence fused to PRO-C-MG.2, PRO-C-MG.12, PRO-C-MG.45, PRO-C-MG.64 or PRO-C-MG.72 DNA and encoding a specific antibody epitope.

5. Purification of Polypeptide

Forms of PRO-C-MG.2, PRO-C-MG.12, PRO-C-MG.45, PRO-C-MG.64 or PRO-C-MG.72 can be recovered from culture medium or from host cell lysates. If membrane-bound, it can be released from the membrane using a suitable detergent solution (e.g. Triton-X 100) or by enzymatic cleavage. Cells employed in expression of PRO-C-MG.2, PRO-C-MG.12, PRO-C-MG.45, PRO-C-MG.64 or PRO-C-MG.72 can be disrupted by various physical or chemical means, such as freeze-thaw cycling, sonication, mechanical disruption, or cell lysing agents.

It can be desired to purify PRO-C-MG.2, PRO-C-MG.12, PRO-C-MG.45, PRO-C-MG.64 or PRO-C-MG.72 from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO-C-MG.2, PRO-C-MG.12, PRO-C-MG.45, PRO-C-MG.64 or PRO-C-MG.72. Various methods of protein purification can be employed and such methods are known in the art and described for example in Deutscher, Methods in Enzymology, 182 (1990); Scopes, Protein Purification: Principles and Practice. Springer-Verlag, New York (1982). The purification step(s) selected will depend, for example, on the nature of the production process used and the particular PRO-C-MG.2, PRO-C-MG.12, PRO-C-MG.45, PRO-C-MG.64 or PRO-C-MG.72 produced.

E. Uses for PRO-C-MG.2, PRO-C-MG.12, PRO-C-MG.45, PRO-C-MG.64 or PRO-C-MG.72

Nucleotide sequences (or their complement) encoding PRO-C-MG.2, PRO-C-MG.12, PRO-C-MG.45, PRO-C-MG.64 or PRO-C-MG.72 have various applications in the art of molecular biology, including uses as hybridization probes, in chromosome and gene mapping and in the generation of anti-sense RNA, DNA, and PNA (peptide nucleic acids). PRO-C-MG.2, PRO-C-MG.12, PRO-C-MG.45, PRO-C-MG.64 or PRO-C-MG.72 nucleic acid will also be useful for the preparation of PRO-C-MG.2, PRO-C-MG.12, PRO-C-MG.45, PRO-C-MG.64 or PRO-C-MG.72 polypeptides by the recombinant techniques described herein. Full-length or fragments of a PRO-C-MG.2, PRO-C-MG.12, PRO-C-MG.45, PRO-C-MG.64 or PRO-C-MG.72 polypeptide coding sequence find use as, for example, hybridization probes or for encoding a PRO-C-MG.2, PRO-C-MG.12, PRO-C-MG.45, PRO-C-MG.64 or PRO-C-MG.72 polypeptide or fragment thereof that can optionally encode a polypeptide comprising a binding site for an anti-PRO-C-MG.2, PRO-C-MG.12, PRO-C-MG.45, PRO-C-MG.64 or PRO-C-MG.72 antibody.

The full-length native sequence PRO-C-MG.2, PRO-C-MG.12, PRO-C-MG.45, PRO-C-MG.64 or PRO-C-MG.72 gene (SEQ ID NO:1 or SEQ ID NO:3, respectively), or portions thereof, can be used as hybridization probes for a cDNA library to isolate the full-length PRO-C-MG.2, PRO-C-MG.12, PRO-C-MG.45, PRO-C-MG.64 or PRO-C-MG.72 cDNA or to isolate still other cDNAs (for instance, those encoding naturally-occurring variants of PRO-C-MG.2, PRO-C-MG.12, PRO-C-MG.45, PRO-C-MG.64 or PRO-C-MG.72 or PRO-C-MG.2, PRO-C-MG.12, PRO-C-MG.45, PRO-C-MG.64 or PRO-C-MG.72 from other species) which have a desired sequence identity to the PRO-C-MG.2, PRO-C-MG.12, PRO-C-MG.45, PRO-C-MG.64 or PRO-C-MG.72 sequence disclosed in SEQ ID NO:1 or SEQ ID NO:3, respectively. The hybridization probes can be derived from at least partially novel regions of the nucleotide sequence of SEQ ID NO:1 or SEQ ID NO:3, respectively, wherein those regions can be determined without undue experimentation or from genomic sequences including promoters, enhancer elements and introns of native sequence PRO-C-MG.2, PRO-C-MG.12, PRO-C-MG.45, PROC-MG.64 or PRO-C-MG.72.

Such nucleic acid fragments are usually at least about 20 nucleotides in length, preferably at least about 30 nucleotides in length, more preferably at least about 40 nucleotides in length, yet more preferably at least about 50 nucleotides in length, yet more preferably at least about 60 nucleotides in length, yet more preferably at least about 70 nucleotides in length, yet more preferably at least about 80 nucleotides in length, yet more preferably at least about 90 nucleotides in length, yet more preferably at least about 100 nucleotides in length, yet more preferably at least about 110 nucleotides in length, yet more preferably at least about 120 nucleotides in length, yet more preferably at least about 130 nucleotides in length, yet more preferably at least about 140 nucleotides in length, yet more preferably at least about 150 nucleotides in length, yet more preferably at least about 160 nucleotides in length, yet more preferably at least about 170 nucleotides in length, yet more preferably at least about 180 nucleotides in length, yet more preferably at least about 190 nucleotides in length, yet more preferably at least about 200 nucleotides in length, yet more preferably at least about 250 nucleotides in length, yet more preferably at least about 300 nucleotides in length, yet more preferably at least about 350 nucleotides in length, yet more preferably at least about 400 nucleotides in length, yet more preferably at least about 450 nucleotides in length, yet more preferably at least about 500 nucleotides in length, yet more preferably at least about 600 nucleotides in length, yet more preferably at least about 700 nucleotides in length, yet more preferably at least about 800 nucleotides in length, yet more preferably at least about 900 nucleotides in length and yet more preferably at least about 1000 nucleotides in length, wherein in this context the term “about” means the referenced nucleotide sequence length plus or minus 10% of that referenced length. In a preferred embodiment, the nucleotide sequence fragment is derived from any coding region of the nucleotide sequence shown in SEQ ID NO: 1 or SEQ ID NO:3, respectively. In one embodiment the fragment size range is from 20 to 50 nucleotides, which is particularly useful for probe or antisense use. It is noted that novel fragments of a PRO-C-MG.2, PRO-C-MG.12, PRO-C-MG.45, PRO-C-MG.64 or PRO-C-MG.72 polypeptide-encoding nucleotide sequence can be determined in a routine manner by aligning the PRO-C-MG.2, PRO-C-MG.12, PRO-C-MG.45, PRO-C-MG.64 or PRO-C-MG.72 polypeptide-encoding nucleotide sequence with other known nucleotide sequences using any of a number of well known sequence alignment programs and determining which PRO-C-MG.2, PRO-C-MG.12, PRO-C-MG.45, PRO-C-MG.64 or PRO-C-MG.72 polypeptide-encoding nucleotide sequence fragment(s) are novel. All of such PRO-C-MG.2, PRO-C-MG.12, PRO-C-MG.45, PRO-C-MG.64 or PRO-C-MG.72 polypeptide-encoding nucleotide sequences are contemplated herein and can be determined without undue experimentation. Also contemplated are the PRO-C-MG.2, PRO-C-MG.12, PRO-C-MG.45, PRO-C-MG.64, or PRO-C-MG.72 polypeptide fragments encoded by these nucleotide molecule fragments, preferably those PRO-C-MG.2, PRO-C-MG.12, PRO-C-MG.45, PRO-C-MG.64 or PRO-C-MG.72 polypeptide fragments that comprise a binding site for an anti-PRO-C-MG.2, PRO-C-MG.12, PRO-C-MG.45, PRO-C-MG.64 or PRO-C-MG.72 antibody.

By way of example, a screening method will comprise isolating the coding region of the PRO-C-MG.2, PRO-C-MG.12, PRO-C-MG.45, PRO-C-MG.64 or PRO-C-MG.72 gene using the known DNA sequence to synthesize a selected probe of about 40 bases. Hybridization probes can be labeled by a variety of labels, including radionucleotides such as ³²p or ³⁵S, or enzymatic labels such as alkaline phosphatase coupled to the probe via avidin/biotin coupling systems. Labeled probes having a sequence complementary to that of the PRO-C-MG.2, PRO-C-MG.12, PRO-C-MG.45, PRO-C-MG.64 or PRO-C-MG.72 gene of the present invention can be used to screen libraries of human cDNA, genomic DNA or mRNA to determine which members of such libraries the probe hybridizes to. Hybridization techniques are described in further detail in the Examples below.

Any EST sequences disclosed in the present application can similarly be employed as probes, using the methods disclosed herein.

Other useful fragments of the PRO-C-MG.2, PRO-C-MG.12, PRO-C-MG.45, PRO-C-MG.64 or PRO-C-MG.72 nucleic acids include antigene (antisense or sense) oligonucleotides comprising a singe-stranded nucleic acid sequence (either RNA or DNA) capable of binding to target PRO-C-MG.2, PRO-C-MG.12, PRO-C-MG.45, PRO-C-MG.64 or PRO-C-MG.72 mRNA (sense) or PRO-C-MG.2, PRO-C-MG.12, PRO-C-MG.45, PRO-C-MG.64 or PRO-C-MG.72 DNA (antisense) sequences. Antigene compounds comprise a fragment of the sequence of PRO-C-MG.2, PRO-C-MG.12, PRO-C-MG.45, PRO-C-MG.64 or PRO-C-MG.72 gene as discussed above and in more detail below. The fragment can include either 5′ or 3′ non-coding regions.

PRO-C-MG.2, PRO-C-MG.12, PRO-C-MG.45, PRO-C-MG.64 or PRO-C-MG.72 oligonucleotides and probes can also be employed in PCR techniques to generate a pool of sequences for identification of closely related PRO-C-MG.2, PRO-C-MG.12, PRO-C-MG.45, PRO-C-MG.64 or PRO-C-MG.72 coding sequences.

Nucleotide sequences encoding a PRO-C-MG.2, PRO-C-MG.12, PRO-C-MG.45, PRO-C-MG.64 or PRO-C-MG.72 can also be used to construct hybridization probes for mapping the gene which encodes that PRO-C-MG.2, PRO-C-MG.12, PRO-C-MG.45, PRO-C-MG.64 or PRO-C-MG.72 and for the genetic analysis of individuals with genetic disorders. The nucleotide sequences provided herein can be mapped to a chromosome and specific regions of a chromosome using known techniques, such as in situ hybridization, linkage analysis against known chromosomal markers, and hybridization screening with libraries.

When the coding sequences for PRO-C-MG.2, PRO-C-MG.12, PRO-C-MG.45, PRO-C-MG.64 or PRO-C-MG.72 encode a protein which binds to another protein, the PRO-C-MG.2, PRO-C-MG.12, PRO-C-MG.45, PRO-C-MG.64 or PRO-C-MG.72 can be used in assays to identify the other proteins or molecules involved in the binding interaction. By such methods, inhibitors of the binding interaction can be identified. Proteins involved in such binding interactions can also be used to screen for peptide or small molecule inhibitors or agonists of the binding interaction. Also, the receptor PRO-C-MG.2, PRO-C-MG.12, PRO-C-MG.45, PRO-C-MG.64 or PRO-C-MG.72 can be used to isolate correlative ligand(s). Screening assays can be designed to find lead compounds that mimic the biological activity of a native PRO-C-MG.2, PRO-C-MG.12, PRO-C-MG.45, PRO-C-MG.64 or PRO-C-MG.72 or a receptor for PRO-C-MG.2, PRO-C-MG.12, PRO-C-MG.45, PRO-C-MG.64 or PRO-C-MG.72. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art. Such high- and ultra-high throughput assays are can also be used to test antisense molecules. One such assay includes the use of reporter molecules, such as beta-lactamase, in which a beta-lactamase expression cassette is integrated into the test cell genome in such a way that modulation of the biological response of interest, e.g. tube formation, is reflected as modulation of beta-lactamase activity, preferably measured by fluorescence (e.g., see WO 98/13353 and WO 98/52047). Nucleic acids which encode PRO-C-MG.2, PRO-C-MG.12, PRO-C-MG.45, PRO-C-MG.64 or PRO-C-MG.72 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding PRO-C-MG.2, PRO-C-MG.12, PRO-C-MG.45, PRO-C-MG.64 or PRO-C-MG.72 can be used to clone genomic DNA encoding PRO-C-MG.2, PRO-C-MG.12, PRO-C-MG.45, PRO-C-MG.64 or PRO-C-MG.72 in accordance with established techniques and the genomic sequences used to generate transgenic animals that contain cells which express DNA encoding PRO-C-MG.2, PRO-C-MG.12, PRO-C-MG.45, PRO-C-MG.64 or PRO-C-MG.72. Methods for generating transgenic animals, particularly animals such as mice or rats, have become conventional in the art and are described, for example, in U.S. Pat. Nos. 4,736,866 and 4,870,009. Typically, particular cells would be targeted for PRO-C-MG.2, PRO-C-MG.12, PRO-C-MG.45, PRO-C-MG.64 or PRO-C-MG.72 transgene incorporation with tissue-specific enhancers. Transgenic animals that include a copy of a transgene encoding PRO-C-MG.2, PRO-C-MG.12, PRO-C-MG.45, PRO-C-MG.64 or PRO-C-MG.72 introduced into the germ line of the animal at an embryonic stage can be used to examine the effect of increased expression of DNA encoding PRO-C-MG.2, PRO-C-MG.12, PRO-C-MG.45, PRO-C-MG.64 or PRO-C-MG.72. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of PRO-C-MG.2, PRO-C-MG.12, PRO-C-MG.45, PRO-C-MG.64 or PRO-C-MG.72 can be used to construct a PRO-C-MG.2, PRO-C-MG.12, PRO-C-MG.45, PRO-C-MG.64 or PRO-C-MG.72 “knock out” animal which has a defective or altered gene encoding PRO-C-MG.2, PRO-C-MG.12, PRO-C-MG.45, PRO-C-MG.64 or PRO-C-MG.72 as a result of homologous recombination between the endogenous gene encoding PRO-C-MG.2, PRO-C-MG.12, PRO-C-MG.45, PRO-C-MG.64 or PRO-C-MG.72 and altered genomic DNA encoding PRO-C-MG.2, PRO-C-MG.12, PRO-C-MG.45, PRO-C-MG.64 or PRO-C-MG.72 introduced into an embryonic stem cell of the animal. For example, cDNA encoding PRO-C-MG.2, PRO-C-MG.12, PRO-C-MG.45, PRO-C-MG.64 or PRO-C-MG.72 can be used to clone genomic DNA encoding PRO-C-MG.2, PRO-C-MG.12, PRO-C-MG.45, PRO-C-MG.64 or PRO-C-MG.72 in accordance with established techniques. A portion of the genomic DNA encoding PRO-C-MG.2, PRO-C-MG.12, PRO-C-MG.45, PRO-C-MG.64 or PRO-C-MG.72 can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the PRO-C-MG.2, PRO-C-MG.12, PRO-C-MG.45, PRO-C-MG.64 or PRO-C-MG.72 polypeptide.

Nucleic acid encoding the PRO-C-MG.2, PRO-C-MG.12, PRO-C-MG.45, PRO-C-MG.64 or PRO-C-MG.72 polypeptides can also be used in gene therapy. In gene therapy applications, genes are introduced into cells in order to achieve in vivo synthesis of a therapeutically effective genetic product, for example for replacement of a defective gene. Alternatively, in vivo synthesis of an antisense form of the target gene can reduce unwanted target gene expression, such as in the case of tumors, viral infections, or conditions involving gene overexpression. “Gene therapy” includes both conventional gene therapy where a lasting effect is achieved by a acute treatment (e.g., a single treatment), and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups such as in peptide nucleic acids (PNAs).

There are a variety of techniques available for introducing nucleic acids, including antigene oligonucletides, into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. In one embodiment, in vivo gene transfer techniques include transfection with viral (typically retroviral, such as adenovirus, lentivirus, Herpes simplex 1 virus, or adeno-associated virus (AAV)) vectors, viral coat protein-liposome mediated transfection (Dzau et al., Trends in Biotechnology 11:205-210 [1993]), and lipid-based systems (for example, DOTMA, DOPE, and DC-Chol; see, e.g., Tonkinson et al., Cancer Investigation 14(1): 54-65 (1996)). WO 99/22772 discloses particularly useful liposomes for use with antigene oligonucleotides. A viral vector such as a retroviral vector includes at least one transcriptional promoter/enhancer or locus-defining element(s), or other elements that control gene expression by other means such as alternate splicing, nuclear RNA export, or post-translational modification of messenger. In addition, a viral vector such as a retroviral vector includes a nucleic acid molecule that, when transcribed in the presence of a gene encoding PRO-C-MG.2, PRO-C-MG.12, PRO-C-MG.45, PRO-C-MG.64 or PRO-C-MG.72 polypeptide, is operably linked thereto and acts as a translation initiation sequence. Such vector constructs also include a packaging signal, long terminal repeats (LTRs) or portions thereof, and positive and negative strand primer binding sites appropriate to the virus used (if these are not already present in the viral vector). In addition, such vector typically includes a signal sequence for secretion of the PRO-C-MG.2, PRO-C-MG.12, PRO-C-MG.45, PRO-C-MG.64 or PRO-C-MG.72 polypeptide from a host cell in which it is placed. Preferably the signal sequence for this purpose is a mammalian signal sequence. Should the PRO-C-MG.2, PRO-C-MG.12, PRO-C-MG.45, PRO-C-MG.64 or PRO-C-MG.72 polypeptide contain an C-terminal or internal translocation peptide, it is preferable to delete it or inactivate it by mutation to avoid interference with the heterologous secretion signal peptide activity. Optionally, the vector construct may also include a signal that directs polyadenylation, as well as one or more restriction sites and a translation termination sequence. By way of example, such vectors will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis can be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87,3410-3414 (1990). For review of gene marking and gene therapy protocols see Anderson et al., Science 256. 808-813 (1992).

Chromosome Markers. The sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

Briefly, sequences can be mapped to chromosomes by preparing PCR primers (preferably 15-25 bp) from the cDNA. Computer analysis for the 3′-untranslated region is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes, and preselection by hybridization to construct chromosome-specific cDNA libraries.

Fluorescence in situ hybridization (FISH) of a cDNA clone to a metaphase chromosomal spread can be used to provide a precise chromosomal location in one step. This technique can be used with cDNA as short as 500 or 600 bases; however, clones larger than 2,000 bp have a higher likelihood of binding to a unique chromosomal location with sufficient signal intensity for simple detection. FISH requires use of the clones from which the gene encoding the PRO-C-MG.2, PRO-C-MG.12, PRO-C-MG.45, PRO-C-MG.64 or PRO-C-MG.72 polypeptide was derived, and the longer the better. For example, 2,000 bp is good, 4,000 bp is better, and more than 4,000 is probably not necessary to get good results a reasonable percentage of the time. For a review of this technique, see, Verma et al., Human Chromosomes: a Manual of Basic Techniques (Pergamon Press, New York, 1988).

Once a sequence has been mapped to a precise chromosomal location, the physical position of the sequence on the chromosome can be correlated with genetic map data. Such data are found, for example, in V. McKusick, Mendelian Inheritance in Man (available online through Johns Hopkins University Welch Medical Library). The relationship between genes and diseases that have been mapped to the same chromosomal region is then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

The PRO-C-MG.2, PRO-C-MG.12, PRO-C-MG.45, PRO-C-MG.64 or PRO-C-MG.72 polypeptides and nucleic acid molecules of the present invention can also be used for tissue typing, wherein the PRO-C-MG.2, PRO-C-MG.12, PRO-C-MG.45, PRO-C-MG.64 or PRO-C-MG.72 polypeptides of the present invention can be differentially expressed in one tissue as compared to another. PRO-C-MG.2, PRO-C-MG.12, PRO-C-MG.45, PRO-C-MG.64 or PRO-C-MG.72 nucleic acid molecules will find use for generating probes for PCR, Northern analysis, Southern analysis and Western analysis.

The PRO-C-MG.2, PRO-C-MG.12, PRO-C-MG.45, PRO-C-MG.64 or PRO-C-MG.72 polypeptides described herein can also be employed as molecular weight markers for protein electrophoresis purposes.

F. PRO-C-MG.2, PRO-C-MG.12, PRO-C-MG.45, PRO-C-MG.64 or PRO-C-MG.72 Antigene Compounds

PRO-C-MG.2, PRO-C-MG.12, PRO-C-MG.45, PRO-C-MG.64 or PRO-C-MG.72 nucleic acids include antigene compounds, particularly oligonucleotides, for use in modulating the function of PRO-C-MG.2, PRO-C-MG.12, PRO-C-MG.45, PRO-C-MG.64 or PRO-C-MG.72, modulating the amount of PRO-C-MG.2, PRO-C-MG.12, PRO-C-MG.45, PRO-C-MG.64 or PRO-C-MG.72 produced by the cell, and ultimately modulating the biological processes or responses in which PRO-C-MG.2, PRO-C-MG.12, PRO-C-MG.45, PRO-C-MG.64 or PRO-C-MG.72 is critical. This can be accomplished by providing antigene compounds which specifically hybridize with one or more nucleic acids encoding PRO-C-MG.2, PRO-C-MG.12, PRO-C-MG.45, PRO-C-MG.64 or PRO-C-MG.72. As used herein, the terms “target nucleic acid” and “nucleic acid encoding PRO-C-MG.2, PRO-C-MG.12, PRO-C-MG.45, PRO-C-MG.64 or PRO-C-MG.72” encompass DNA encoding PRO-C-MG.2, PRO-C-MG.12, PRO-C-MG.45, PRO-C-MG.64 or PRO-C-MG.72 (e.g., genomic DNA), RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense” technology, however, is now more broadly referred to as “antigene” technology, which expressly includes both sense and antisense sequences and is used herein interchangeably with “antisense.” Antigene compounds include peptide nucleic acids and ribozymes. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of PRO-C-MG.2, PRO-C-MG.12, PRO-C-MG.45, PRO-C-MG.64 or PRO-C-MG.72. In the context of the present invention, “modulation” means either an increase (stimulation) or a decrease (inhibition) in the expression of a gene. In the context of the present invention, inhibition is the preferred form of modulation of gene expression and mRNA is a preferred target.

In the present invention, the target is a nucleic acid molecule encoding PRO-C-MG.2, PRO-C-MG.12, PRO-C-MG.45, PRO-C-MG.64 or PRO-C-MG.72. Methods are available in the art to rapidly determine (within about a week) a site or sites within this gene for the antigene interaction to occur such that the desired effect, e.g., detection or modulation of expression of PRO-C-MG.2, PRO-C-MG.12, PRO-C-MG.45, PRO-C-MG.64 or PRO-C-MG.72, will result. A preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding PRO-C-MG.2, PRO-C-MG.12, PRO-C-MG.45, PRO-C-MG.64 or PRO-C-MG.72, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, can also be targeted effectively. Other target regions include the 5′ untranslated region (5′UTR), which is the portion of an mRNA in the 5′ direction from the translation initiation codon and includes nucleotides between the 5′ cap site and the translation initiation codon of an mRNA or corresponding nucleotides on the gene, and the 3′ untranslated region (3′UTR), which is the portion of an mRNA in the 3′ direction from the translation termination codon and thus includes nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region is also a preferred target region.

While some eukaryotic mRNA transcripts are directly translated, many contain one or more regions known as “introns,” which are excised from a transcript before it is translated. The remaining (and therefore translated) regions are known as “exons” and are spliced together to form a continuous mRNA sequence. When present, mRNA splice sites, i.e., intron-exon junctions, are also preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Introns are also effective target regions for antigene compounds targeted, for example, to DNA or pre-mRNA.

Once one or more target sites have been identified, using techniques in the art, oligonucleotides are chosen which are sufficiently complementary to the PRO-C-MG.2, PRO-C-MG.12, PRO-C-MG.45, PRO-C-MG.64 or PRO-C-MG.72 gene target, i.e., hybridize sufficiently well and with sufficient specificity, to give the desired effect. The ability to derive an antisense or a sense oligonucleotide, based upon a cDNA sequence encoding a given protein is described in, for example, Stein and Cohen (Cancer Res. 48:2659 (1988)) and van der Krol et al. (BioTechniques 6:958 (1988)). For example, targeting sites can be rapidly determined using combinatorial libraries, preferably in microarrays. Synthesis of peptide nucleic acid combinatorial libraries is disclosed in U.S. Pat. No. 5,864,010. Antisense or sense oligonucleotides include PNAs or other molecules having modified backbones or modified nucleosides so long as they are designed upon and specific for a PRO-C-MG.2, PRO-C-MG.12, PRO-C-MG.45, PRO-C-MG.64 or PRO-C-MG.72 nucleic acid sequence.

The sequence of a PRO-C-MG.2, PRO-C-MG.12, PRO-C-MG.45, PRO-C-MG.64 or PRO-C-MG.72 antigene compound need not be 100% complementary to that of its target nucleic acid to be specifically hybridizable, although 100% complementarity is preferred. An antigene compound is specifically hybridizable when binding of the compound to the target PRO-C-MG.2, PRO-C-MG.12, PRO-C-MG.45, PRO-C-MG.64 or PRO-C-MG.72 DNA or RNA molecule interferes with the normal function of the target DNA or RNA to cause a loss of utility, and there is a sufficient degree of complementarity to avoid non-specific binding of the PRO-C-MG.2, PRO-C-MG.12, PRO-C-MG.45, PRO-C-MG.64 or PRO-C-MG.72 antigene compound to non-target sequences under conditions in which specific binding is desired, i.e., under physiological conditions in the case of in vivo assays or therapeutic treatment, and in the case of in vitro assays, under conditions in which the assays are performed.

Methods for administration of antigene compounds to a variety of cells, including HUVEC, in order to modulate target gene function are known (e.g., Ackermann et al., J. Biol. Chem. 274(16):11245-52 (1999)).

The specificity and sensitivity of antigene is particularly suited for therapeutic uses. Antigene oligonucleotides have been employed as therapeutic moieties in the treatment of disease states in animals and man. Antigene oligonucleotides have been safely and effectively administered to humans and numerous clinical trials are presently underway. Antisense oligonucleotides have demonstrated acceptable safety and toxicity profiles in both animals and humans. Numerous antisense molecules are in Phase II and Phase III trials. An antisense compound has been approved and is marketed for treatment of CMV-induced retinitis. As a class, antisense molecules have been proven safe in animals and humans for systemic delivery. It has thus been established that antigene therapy can be a useful therapeutic modality that can be configured to be useful in treatment regimes for treatment of cells, tissues and animals, especially humans. Methods for testing toxicity and efficacy in animal models are thus well-established in the art.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as for example. enhanced cellular uptake, enhanced affinity for nucleic acid target and increased stability in the presence of nucleases.

The antigene compounds in accordance with this invention preferably comprise from about 5 to about 60 nuclcobases. Particularly preferred are antigene oligonucleotides comprising from about 8 to about 30 nuclobases (i.e. from about 8 to about 30 linked nucleosides), and most preferably from about 15 to about 25 nucleosides. Sequences of 17-18 bases are of special interest since this is the estimated length of unique sequences in tile human genome. As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the intemucleoside backbone of the oligonucleotide. The normal linkage or backbone of RNA and DNA is a 3′ to 5′ phosphodiester linkage.

Accordingly, binding of antigene oligonucleotides, either antisense or sense oligonucleotides, to target nucleic acid sequences results in the formation of duplexes or triplexes that block transcription or translation of the target sequence by one of several means, including enhanced degradation of the duplexes, premature termination of transcription or translation, or by other means. The antisense oligonucleotides thus can be used to block expression of PRO-C-MG.2, PRO-C-MG.12, PRO-C-MG.45, PRO-C-MG.64 or PRO-C-MG.72 proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences. Other examples of sense or antisense oligonucleotides include those oligonucleotides which are covalently linked to organic moieties, such as those described in WO 90/10048, and other moieties that increase affinity of the oligonucleotide for a target nucleic acid sequence, such as poly-(L-lysine). Further still, intercalating agents, such as ellipticine, and alkylating agents or metal complexes can be attached to sense or antisense oligonucleotides to modify binding specificities of the antisense or sense oligonucleotide for the target nucleotide sequence as discussed below.

Specific examples of preferred PRO-C-MG.2, PRO-C-MG.12, PRO-C-MG.45, PRO-C-MG.64 or PRO-C-MG.72 antigene compounds include oligonucleotides containing modified backbones or non-natural intemucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their intemucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. Representative United States patents that teach the preparation of the phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Preferred modified oligonucleotide backbones that do not include a phosphorus atom therein have backbones that are formed by short chain alkyl or cycloalkyl intemucleoside linkages, mixed heteroatom and alkyl or cycloalkyl internucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Representative United States patents that teach the preparation of these oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent solubility, membrane-traversing, and hybridization properties, is referred to as a peptide nucleic acid (PNA; Nielsen et al., Science 254:1497-1500 (1991)). In PNA compounds, the sugar-backbone is replaced with an amide containing backbone, e.g., an aminoethylglycine backbone. The nucleobases can be retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, and PCT publication No. WO 97/33551, each of which is herein incorporated by reference. PNA compounds recognize and bind sequence-selectively and strand-selectively to double-stranded DNA (dsDNA), which is accomplished via strand displacement, in which the PNA binds via Watson-Crick binding to its complementary strand and extrudes the other strand in a virtually single-stranded conformation. PNA compounds also recognize and bind sequence-selectively to single-stranded DNA (ssDNA) and to RNA. This recognition by PNA of RNA, ssDNA or dsDNA can take place in sequences at least 5 bases long. A more preferred recognition sequence length is 5 to 60 base pairs long, and more preferably 8 to 30 base pairs long, and most preferably from about 15 to about 25 nucleosides. For therapeutic use of PNA compounds the targets of the PNA compounds would generally be double stranded DNA—in which case the PNA is effective in both the sense and antisense forms—and RNA. For diagnostic use, investigations methods and reagents where DNA is isolated outside of a cell, the DNA can be denatured to single stranded DNA and use of the PNA compound would be targeted to such single stranded DNA as well as RNA.

PNA compounds useful to effect binding to RNA, ssDNA and dsDNA and to form duplex and triplex complexes are polymeric strands formed from a polyamide, polythioamide, polysulfinamide or polysulfonamide backbone with a plurality of ligands located at spaced locations along the backbone, at least some of the ligands capable of hydrogen bonding with other ligands either on the compounds or nucleic acid targets. The amino acids which form the backbone may be identical or different, but those based on 2-aminoethyl-glycine are preferred. In some cases it may be of interest to attach ligands at either terminus to modulate the binding characteristics of the PNAs. Representative ligands include DNA intercalators, which improve dsDNA binding or basic groups, such as lysine or polylysine, which strengthen the binding of the PNA due to electrostatic interaction. To decrease electrostatic repulsion charged groups such as carboxyl and sulfo groups could be used. Oligonucleotides and/or oligonucleoside can be covalently bound to either terminal positions to form chimeras containing PNA portions and oligonucleotide and/or oligonucleoside portions. Nucleosides and/or nucleotides (mono, di or tri-phosphates) also can be attached to the terminal positions. Moieties can also be located on non-terminal positions. In one embodiment, the PNA oligomers are conjugated to low molecular weight effector ligands such as ligands having nuclease activity or alkylating activity or reporter ligands (fluorescent, spin labels, radioactive, protein recognition ligands, for example, biotin or haptens). In another embodiment, the PNAs are conjugated to peptides or proteins, where the peptides have signaling activity and the proteins are, for example, enzymes, transcription factors or antibodies. Also, the PNAs can be attached to water-soluble or water-insoluble polymers. In yet another embodiment, the PNAs are conjugated to oligonucleotides or carbohydrates. When desired a PNA oligomer can be synthesized onto a moiety (e.g., a peptide chain, reporter, intercalator or other type of ligand-containing group) attached to a solid support.

In a further embodiment, PNA compounds also can be used as PRO-C-MG.2, PRO-C-MG.12, PRO-C-MG.45, PRO-C-MG.64 or PRO-C-MG.72 gene-sequence specific gene activators and synthetic transcription factors, useful for selectively up-regulating PRO-C-MG.2, PRO-C-MG.12, PRO-C-MG.45, PRO-C-MG.64 or PRO-C-MG.72. Transcription initiation by RNA polymerase involves the sequence specific recognition of the double-stranded DNA promoter either by the polymerase itself or by auxiliary transcription factors. Subsequently a transcription initiation open complex is formed in which about 12 base pairs of the DNA helix are melted, which exposes the bases of the template strand for base pairing with the RNA strand being synthesized. It has been demonstrated that an E. coli phage T7 RNA polymerase can utilize synthetic “RNA/DNA bubble duplex” complexes containing an RNA/DNA duplex and a single-stranded DNA D-loop for transcription elongation. In addition, homopyrimidine PNAs also form D-loop structures when binding to complimentary double-stranded DNA by strand displacement, structures that behave like RNA/DNA open complex structures and are recognized by RNA polymerase.

Preferred embodiments of the invention are PRO-C-MG.2, PRO-C-MG.12, PRO-C-MG.45, PRO-C-MG.64 or PRO-C-MG.72 oligonucleotides with phosphorothioate backbones and oligonucleosides with heteroatom backbones, and in particular —CH2-NH—O—CH2-, —CH2-N(CH3)-O—CH2- [known as a methylene (methylimino) or MM1 backbone], —CH2-O—N(CH3)-CH2-, —CH2-N(CH3)-N(CH3)-CH2- and —O—N(CH3)-CH2-CH2- [wherein the native phosphodiester backbone is represented as —O—P—O—CH2-] of the above referenced U.S. Pat. No. 5,489,677, and the amide backbones of U.S. Pat. No. 5,602,240. Also preferred are oligonucleotides having morpholino backbone structures as described in U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH2)[n]O][m]CH3, O(CH2)[n]OCH3, O(CH2)[n]NH2, O(CH2)[n]CH3, O(CH2)[n]ONH2, and O(CH2)[n]ON[(CH2)[n]CH3)]2, where n and m are from 1 to about 10. Other preferred oligonucleotides comprise one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharrnacodynamic properties of an oligonucleotide, and other substituents having similar properties. A preferred modification includes 2′-methoxyethoxy (2′-O—CH2CH2OCH3, also known as 2′-O(2-methoxyethyl) or 2′-MOE) (Martin et al., Helv. Chim. Acta, 78:486-504 (1995); McKay et al., J. Biol. Chem. 274(3):1715-22 (1999)) i.e., an alkoxyalkoxy group. The incorporation of 2′-O-(2-methoxyethyl chemistry provides a number of significant improvements in oligonucleotide characteristics, including an increase in hybridization affinity toward a complementary RNA (1.5° C. per modification) and an increase in resistance toward both 3′-exonuclease and intracellular nucleases. These improvements result in a substantial increase in oligonucleotide potency (e.g., >20-fold after 72 h). A further preferred modification includes 2′-dimethylaminooxyethoxy, i.e., a O(CH2)2ON(CH3)2 group, also known as 2′-DMAOE.

Other preferred modifications include 2′-methoxy (2′-O—CH3), 2′-aminopropoxy (2′-OCH2CH2CH2NH2) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adeniline (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, in “The Concise Encyclopedia Of Polymer Science And Engineering,” pages 858-859, Kroschwitz, ed. John Wiley & Sons, (1990), in Englisch et al., Angewandte Chemie, International Edition, 30:613 (1991), and by Sanglivi (Antisense Research and Applications, Chapter 15, pages 289-302, Crooke and Lebleu, ed., CRC Press, (1993)). Nucleobases that are particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. The 5-methylcytosine substitutions have been shown to increase nucleic acid duplex stability by 0.6-1.2° C. (Sanghvi, Id. at pp. 276-278) and are presently preferred base substitutions, even more particularly when combined with 2′-O-methoxyethyl sugar modifications. Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of the PRO-C-MG.2, PRO-C-MG.12, PRO-C-MG.45, PRO-C-MG.64 or PRO-C-MG.72 oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 86:6553-6556 (1989)), cholic acid (Manoharan et al., Bioorg. Med. Chem. Let 4:1053-1060 (1994)), a thioether, e.g., hexyl-S-tritylthiol (Manoharan et al., Ann. N. Y. Acad. Sci. 660:306-309 (1992); Manoharan et al., Bioorg. Med. Chem. Let. 3:2765-2770 (1993)), a thiocholesterol (Oberhauser et al., Nucl. Acids Res. 20:533-538 (1992)), an aliphatic chain, e.g., dodecandiol or undecyl residues (Saison-Behmoaras et al., EMBO J. 10:1111-1118 (1991); Kabanov et al., FEBS Len. 259:327-330 (1990); Svinarchuk et al., Biochimie 75:49-54 (1993)), a phospholipid, e.g., di-hexadecyl-rac-glycerol or triethylammonium 1,2-di-O-hexadecyl-rac-glycero-3-H-phosphonate (Manoharan et al., Tetrahedron Len., 36:3651-3654 (1995); Shea et al., Nucl. Acids Res. 18:3777-3783 (1990), a polyamine or a polyethylene glycol chain (Manoharan et al., Nucleosides & Nucleotides 14:969-973 (1995)), or adamantane acetic acid (Manoharan et al., Tetrahedron Len. 36:3651-3654 (1995), a palmityl moiety (Mishra et al., Biochim. Biophys. Acta 1264:229-237 (1995), or an octadecylamine or hexylamino-carbonyl-oxycholesterol moiety (Crooke et al., J. Pharmacol. Exp. Ther., 277: 923-937 (1996)). Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the modifications described can be incorporated into a single compound or even at a single nucleoside within an oligonucleotide. Accordingly, the present invention also includes PRO-C-MG.2, PRO-C-MG.12, PRO-C-MG.45, PRO-C-MG.64 or PRO-C-MG.72 antigene compounds which are chimeric compounds. By “chimeric PRO-C-MG.2, PRO-C-MG.12, PRO-C-MG.45, PRO-C-MG.64 or PRO-C-MG.72 antigene” compounds or “antigene chimeras” is meant antigene compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide can serve as a substrate for enzymes capable of cleaving RNA:DNA or RNA:RNA hybrids, such as an RNase. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. Chimeric antigene compounds of the invention can be formed as composite structures of two or more oligonucleotides, modified oligonucleotides, oligonucleosides and/or oligonucleotide mimetics as described herein. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers.” Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety. The term “prodrug” indicates a therapeutic agent that is prepared in an inactive form that is converted to an active form (i.e., drug) within the body or cells thereof by the action of endogenous enzymes or other chemicals and/or conditions. Included as PRO-C-MG.2, PRO-C-MG.12, PRO-C-MG.45, PRO-C-MG.64 or PRO-C-MG.72 antigene compounds are their prodrug versions. For example, prodrug versions of the PRO-C-MG.2, PRO-C-MG.12, PRO-C-MG.45, PRO-C-MG.64 or PRO-C-MG.72 oligonucleotides can be prepared as SATE [(S-acetyl-2-thioethyl) phosphate] derivatives according to the methods disclosed in WO 93/24510 or in WO 94/26764.

PNA compounds of the invention can be synthesized by any methodology, including those disclosed in WO 92/20702, WO/92/20703 and U.S. Pat. No. 5,641,625.

The PRO-C-MG.2, PRO-C-MG.12, PRO-C-MG.45, PRO-C-MG.64 or PRO-C-MG.72 antigene oligonucletide compounds of the invention can be conveniently and routinely made through the well-known technique of solid phase synthesis. Any other means for such synthesis known in the art can additionally or alternatively be employed.

The PRO-C-MG.2, PRO-C-MG.12, PRO-C-MG.45, PRO-C-MG.64 or PRO-C-MG.72 antigene compounds of the invention can be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The PRO-C-MG.2, PRO-C-MG.12, PRO-C-MG.45, PRO-C-MG.64 or PRO-C-MG.72 antigene compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “pharmaceutically acceptable salts” refers to physiologically and pharmaceutically acceptable salts of the compounds of the invention: i.e., salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci. 66:1-19 (1977)). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quatemary ammonium cations. Carbonates or hydrogen carbonates are also possible.

For PRO-C-MG.2, PRO-C-MG.12, PRO-C-MG.45, PRO-C-MG.64 or PRO-C-MG.72 oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The PRO-C-MG.2, PRO-C-MG.12, PRO-C-MG.45, PRO-C-MG.64 or PRO-C-MG.72 antigene compounds of the present invention can be utilized for diagnostics, therapeutics, prophylaxis and as research reagents and kits. For therapeutics, an animal, preferably a human, suspected of having a disease or disorder as discussed herein, which can be treated by modulating the expression of PRO-C-MG.2, PRO-C-MG.12, PRO-C-MG.45, PRO-C-MG.64 or PRO-C-MG.72, is treated by administering antigene compounds in accordance with the invention. The compounds of the invention can be utilized in pharmaceutical compositions by adding an effective amount of an antigene compound to a suitable pharmaceutically acceptable diluent or carrier. Use of the antigene compounds and methods of the invention can also be useful prophylactically, e.g., to prevent or delay the desired response.

The PRO-C-MG.2, PRO-C-MG.12, PRO-C-MG.45, PRO-C-MG.64 or PRO-C-MG.72 antigene compounds, as research and diagnostic agents, hybridize to nucleic acids encoding PRO-C-MG.2, PRO-C-MG.12, PRO-C-MG.45, PRO-C-MG.64 or PRO-C-MG.72, enabling sandwich and other assays to easily be constructed. Hybridization of the antigene oligonucleotides of the invention with a nucleic acid encoding PRO-C-MG.2, PRO-C-MG.12, PRO-C-MG.45, PRO-C-MG.64 or PRO-C-MG.72 can be detected by means known in the art. Such means include conjugation of an enzyme to the oligonucleotide, radiolabelling of the oligonucleotide, fluorescence reporters, or any other suitable detection means. Kits using such detection means for detecting the level of PRO-C-MG.2, PRO-C-MG.12, PRO-C-MG.45, PRO-C-MG.64 or PRO-C-MG.72 in a sample can be prepared. PRO-C-MG.2, PRO-C-MG.12, PRO-C-MG.45, PRO-C-MG.64 or PRO-C-MG.72 antigene compounds can be introduced into a cell containing the target nucleic acid sequence by any gene transfer method, including, for example, CaPO₄-mediated DNA transfection, electroporation, or by using gene transfer vectors such as Epstein-Barr virus, and those discussed in detail herein. In brief, in a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641). Sense or antisense oligonucleotides also can be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide can be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase. These and other methods are discussed is more detail herein.

Accordingly, the present invention also includes pharmaceutical compositions and formulations which include the PRO-C-MG.2, PRO-C-MG.12, PRO-C-MG.45, PRO-C-MG.64 or PRO-C-MG.72 antigene compounds of the invention. The pharmaceutical compositions of the present invention are administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. PNAs, administered i.p., have been shown to cross the blood-brain barrier and specifically reduce targeted gene expression (see e.g., Tyler et al., PNAS 96(12):7053-8 (1999)) in vivo.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients. Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions.

The compositions of the present invention may be prepared and formulated as emulsions. Emulsions are typically heterogenous systems of one liquid dispersed in another in the form of droplets usually exceeding 0.1 mu m in diameter. (Idson, in Pharmaceutical Dosage Forms, Lieberman, Rieger and Banker (Eds.), Marcel Dekker, Inc., New York, N.Y., volume 1, p. 199 (1988); Rosoff, in Pharmaceutical Dosage Forms, Lieberman, Rieger and Banker (Eds.), Marcel Dekker, Inc., New York, N.Y., Volume 1, p. 245 (1988); Block in Pharmaceutical Dosage Forms, Lieberman, Rieger and Banker (Eds.), Marcel Dekker, Inc., New York, N.Y., volume 2, p. 335 (1988); Higuchi et al., in Remington's Pharmaceutical Sciences, Mack Publishing Co., Easton, Pa., p. 301 (1985)). Emulsions are often biphasic systems comprising of two immiscible liquid phases intimately mixed and dispersed with each other. In general, emulsions may be either water in oil (w/o) or of the oil in water (o/w) variety. When an aqueous phase is finely divided into and dispersed as minute droplets into a bulk oily phase the resulting composition is called a water in oil (w/o) emulsion. Alternatively, when an oily phase is finely divided into and dispersed as minute droplets into a bulk aqueous phase the resulting composition is called an oil in water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil in water in oil (o/w/o) and water in oil in water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion. Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), Marcel Dekker, Inc., New York, N.Y., volume 1, p. 199 (1988)).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), Marcel Dekker, Inc., New York, N.Y., volume 1, p. 285 (1988); Idson, in Pharmaceutical Dosage Forms, Lieberman, Rieger and Banker (Eds.), Marcel Dekker, Inc., New York, N.Y., volume 1, p. 199 (1988)). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), Marcel Dekker, Inc., New York, N.Y., volume 1, p. 285 (1988)).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montinorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), Marcel Dekker, Inc., New York, N.Y., volume 1, p. 335 (1988); Idson, in Pharmaceutical Dosage Forms, Lieberman, Rieger and Banker (Eds.), Marcel Dekker, Inc., New York, N.Y., volume 1, p. 199 (1988)).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylc cellulose and carboxypropyl cellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), Marcel Dekker, Inc., New York, N.Y., volume 1, p. 199 (1988)). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), Marcel Dekker, Inc., New York, N.Y., volume 1, p. 245 (1988); Idson, in Pharmaceutical Dosage Forms, Lieberman, Rieger and Banker (Eds.), Marcel Dekker, Inc., New York, N.Y., volume 1, p. 199 (1988)). Mineral-oil base laxatives, oil-soluble vitamins and high fat nutritive preparations are among the materials that have commonly been administered orally as o/w emulsions.

Microemulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), Marcel Dekker, Inc., New York, N.Y., volume 1, p. 245 (1988)). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shall, in: Controlled Release of Drugs: Polymers and Aggregate Systems, Rosoff, M., Ed., VCH Publishers, New York, pages 185-215 (1989)). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schon, in Remington's Pharmaceutical Sciences, Mack Publishing Co., Easton, Pa., p. 271 (1985)).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), Marcel Dekker, Inc., New York, N.Y., volume 1, p. 245 (1988); Block, in Pharmaceutical Dosage Forms, Liebennan, Rieger and Banker (Eds.), Marcel Dekker, Inc., New York, N.Y., volume 1, p. 335 (1988)). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and triglycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 11:1385-1390 (1994); Ritschel, Meth. Find. Exp. Clin. Pharmacol. 13:205 (1993)). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 11:1385 (1994); Ho et al., J. Pharm. Sci., 85:138-143 (1996)). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate and penetration enhancers to improve the properties of the formulation and to enhance the absorption of the oligonucleotides and nucleic acids of the present invention. Penetration enhancers used in the microemulsions of the present invention may be classified as belonging to one of five broad categories-surfactants, fatty acids, bile salts, chelating agents, and non-chelating non-surfactants (Lee et al., Critical Reviews in Therapeutic Drug Carrier Systems, p. 92 (1991)), as discussed

Liposomes.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), Marcel Dekker, Inc., New York, N.Y., volume 1, p. 245 (1988)). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 147:980-985 (1987)).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19:269-274 (1992)).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 2:405-410 (1992)). Further, an additional study tested the efficacy of interferon administered as part of a liposomal formulation to the administration of interferon using an aqueous system, and concluded that the liposomal formulation was superior to aqueous administration (du Plessis et al., Antiviral Research 18:259-265 (1992)).

Non-ionic liposomal systems have also been examined to determine their utility in the delivery of drugs to the skin, in particular systems comprising non-ionic surfactant and cholesterol. Non-ionic liposomal formulations comprising Novasome TM I (glyceryl dilaurate/cholesterol/polyoxyethylene-10-stearyl ether) and Novasome TM II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G[M1], or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 223:42 (1987); Wu et al., Cancer Research 53:3765 (1993)).

Various liposomes comprising one or more glycolipids are known in the art. Papahadjopoulos et al. (Ann. N. Y. Acad. Sci. 507:64 (1987)) reported the ability of monosialoganglioside G[M1], galactocerebroside sulfate and phosphatidylinositol to improve blood half-lives of liposomes. These findings were expounded upon by Gabizon et al. (PNAS 85:6949 (1988)). U.S. Pat. No. 4,837,028 and WO 88/04924 disclose liposomes comprising (1) sphingomyelin and (2) the ganglioside G[M1]or a galactocerebroside sulfate ester. U.S. Pat. No. 5,543,152 discloses liposomes comprising sphingomyelin. Liposomes comprising 1,2-sn-dimyristoylphosphatidylcholine are disclosed in WO 97/13499. Synthetic verisons of these molecules are preferred.

Many liposomes comprising lipids derivatized with one or more hydrophilic polymers, and methods of preparation thereof, are known in the art. Sunamoto et al. (Bull Chem. Soc. Jpn. 53:2778 (1980)) described liposomes comprising a nonionic detergent, 2C121 5G, that contains a PEG moiety. Illum et al. (FEBS Lett. 167:79 (1984)) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett. 268:235 (1990)) described experiments demonstrating that liposomes comprising phosphatidylethanolamine (PE) derivatized with PEG or PEG stearate have significant increases in blood circulation half-lives. Blume et al. (Biochimica et Biophysica Acta 1029:91 (1990)) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces. A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y. p. 285 (1988)).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., p. 285 (1988)).

Penetration Enhancers.

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants (Lee et al., Crit. Rev. Ther. Drug Carrier Systems p. 92 (1991)). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Crit. Rev. Ther. Drug Carrier Systems, p. 92 (1991)); and perfluorochemical emulsions, such as FC43. Takahashi et al., J. Pharm. Pharmacol. 40:252 (1988)).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcamitines, acylcholines, C[1-10]-alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Crit. Rev. Ther. Drug Carrier Systems p. 92 (1991); Muranishi, Crit. Rev. Ther. Drug Carrier Systems 7:1-33 (1990); El Hariri et al., J. Pharm. Pharmacol. 44: 651-654 (1992)).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, N.Y. pp. 934-935 (1996)). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, page 92 (1991); Swinyard, Chapter 39 In: Remington's Pharmaceutical Sciences, 18th Ed., Gennaro, ed., Mack Publishing Co., Easton, Pa., pages 782-783 (1990); Muranishi, Critical Reviews in Therapeutic Drug Carrier Systems, 7: 1-33 (1990); Yamamoto et al., J. Pharm. Exp. Ther. 263:25 (1992); Yamashita et al., J. Pharm. Sci. 79:579-583 (1990)).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr. 618:315-339 (1993)). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, page 92 (1991); Muranishi, Critical Reviews in Therapeutic Drug Carrier Systems, 7:1-33 (1990); Buur et al., J. Control Rel. 14:43-51 (1990)).

Nonchelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 7: 1-33 (1990)). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, page 92 (1991)); and non-steroidal anti-inflammatory agents such as diclofenac sodium, indomethacin and phenylbutazone (Yamashita et al., J. Pharm. Pharmacol. 39:621-626 (1987)).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev. 5:115-121 (1995); Takakura et al., Antisense & Nucl. Acid Drug Dev. 6:177-183 (1996)).

Excipients.

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components.

The PRO-C-MG.2, PRO-C-MG.12, PRO-C-MG.45, PRO-C-MG.64 or PRO-C-MG.72 antigene compositions of the present invention can additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Administration.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 ug to 100 g per kg of body weight, and can be given once or more daily, weekly, monthly or yearly, or even once every 2 to 20 years. Preferred dosage is from 0.005 to 35 mg/kg body weight, even more preferred is 0.05 to 20 mg/kg body weight, and yet more preferred is 0.01 to 10 mg/kg body weight Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

G. Screening Assays for Drug Candidates.

This invention encompasses methods of screening compounds to identify those that mimic the PRO-C-MG.2, PRO-C-MG.12, PRO-C-MG.45, PRO-C-MG.64 or PRO-C-MG.72 polypeptide (agonists) or prevent the effect of the PRO-C-MG.2, PRO-C-MG.12, PRO-C-MG.45, PRO-C-MG.64 or PRO-C-MG.72 polypeptide (antagonists). Screening assays for antagonist drug candidates are designed to identify compounds that bind or complex with the PRO-C-MG.2, PRO-C-MG.12, PRO-C-MG.45, PRO-C-MG.64 or PRO-C-MG.72 polypeptides encoded by the genes identified herein, or with a gene and mRNAs encoding PRO-C-MG.2, PRO-C-MG. 12, PRO-C-MG.45, PRO-C-MG.64 or PRO-C-MG.72, or otherwise interfere with the interaction of the encoded polypeptides with other cellular proteins. These screening assays will include assays amenable to high- or ultra-high-throughput screening of chemical libraries, making them particularly suitable for identifying antigene (antisense or sense) and small molecule drug candidates.

Polypeptide-targeted assays can be performed in a variety of formats, including protein-protein binding assays, biochemical screening assays, immunoassays, target nucleic acid binding assays, and cell-based assays, which are well characterized in the art. A drug candidate is contacted with a PRO-C-MG.2, PRO-C-MG.12, PRO-C-MG.45, PRO-C-MG.64 or PRO-C-MG.72 polypeptide encoded by a nucleic acid identified herein under conditions and for a time sufficient to allow these two components to interact.

In binding assays, the interaction is binding and the complex formed can be isolated or detected in the reaction mixture. In a particular embodiment, the PRO-C-MG.2, PRO-C-MG.12, PRO-C-MG.45, PRO-C-MG.64 or PRO-C-MG.72 polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the PRO-C-MG.2, PRO-C-MG.12, PRO-C-MG.45, PRO-C-MG.64 or PRO-C-MG.72 polypeptide and drying. Alternatively, an immobilized antibody, e.g., a monoclonal antibody, specific for the PRO-C-MG.2, PRO-C-MG.12, PRO-C-MG.45, PRO-C-MG.64 or PRO-C-MG.72 polypeptide to be immobilized can be used to anchor it to a solid surface. The assay is perfonned by adding the non-immobilized component, which can be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular PRO-C-MG.2, PRO-C-MG.12, PRO-C-MG.45, PRO-C-MG.64 or PRO-C-MG.72 polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a PRO-C-MG.2, PRO-C-MG.12, PRO-C-MG.45, PRO-C-MG.64 or PRO-C-MG.72 polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo can be added to a third reaction mixture, to serve as positive control. The binding (complex formation) between the test compound and the intra- or extracellular component present in the mixture is monitored as described herein. The formation of a complex in the control reaction(s) but not in the reaction mixture containing the test compound indicates that the test compound interferes with the interaction of the test compound and its reaction partner. A particularly useful assay system is a microarray assay, such as chip upon which a nucleic acid fragment-sequence library—based on the PRO-C-MG.2, PRO-C-MG.12, PRO-C-MG.45, PRO-C-MG.64 or PRO-C-MG.72 gene sequence—is synthesized.

Oligonucleotides or longer fragments derived from any of the polynucleotide sequences described herein can be used as targets in a microarray. The microarray can be used to monitor the expression level of large numbers of genes simultaneously (to produce a transcript image), to identify genetic variants, mutations and polymorphisms, to identify effective nucleic acid binding molecules such as antisense molecules, regulatory proteins, ribosomes or polymerases. This information may be used to determine gene function, to understand the genetic basis of disease, to diagnose disease, to identify therapeutic molecules (e.g., antisense), and to develop, and monitor the activities of therapeutic agents.

In one embodiment, the microarray can be prepared and used according to the methods known in the art, such as those described in WO95/11995 (Chee et al.), Lockhart, D. J., et al. (Nat. Biotech. 14: 1675-1680 (1996)), and Schena, M., et al. (Proc. Natl. Acad. Sci. 93: 10614-10619 (1996)) or in WO 99/24463.

The microarray is preferably composed of a large number of unique, single-stranded nucleic acid sequences, usually either synthetic antisense oligonucleotides or fragments of cDNAs, fixed to a solid support. The oligonucleotides are preferably about 5 to 60 nucleotides in length, more preferably about 8 to 30, even more preferably about 15 to 30 nucleotides in length, even more preferably 15 to 25, and most preferably about 20 to 25 nucleotides in length. For a certain type of microarray, it may be preferable to use oligonucleotides that are only 7 to 10 nucleotides in length. The microarray can contain oligonucleotides which cover the known 5′ (or 3′) sequence or untranslated regions, sequential oligonucleotides which cover the full-length sequence or unique oligonucleotides selected from particular areas along the length of the sequence including untranslated regions. Polynucleotides used in the microarray can be oligonucleotides that are specific to a gene or genes of interest, preferably a PRO-C-MG.2, PRO-C-MG.12, PRO-C-MG.45, PRO-C-MG.64 or PRO-C-MG.72 gene, in which at least a fragment of the sequence is known or that are specific to one or more unidentified cDNAs that are common to a particular cell or tissue type or to a normal, developmental, or disease state. In certain situations, it is appropriate to use pairs of oligonucleotides on a microarray. The pairs will be identical, except for one nucleotide preferably located in the center of the sequence. The second oligonucleotide in the pair (mismatched by one) serves as a control. The number of oligonucleotide pairs may range from 2 to 1,000,000. Microarrays can also contain fragments in DNA duplex form, which are particularly useful in identifying molecules that bind to PRO-C-MG.2, PRO-C-MG.12, PRO-C-MG.45, PRO-C-MG.64 or PRO-C-MG.72 genomic DNA.

For producing oligonucleotides to a known sequence for a microarray, the gene of interest is examined using a computer algorithm which starts at the 5′ or more preferably at the 3′ end of the nucleotide sequence. The algorithm identifies oligomers of defined length that are unique to the gene, have a GC content within a range suitable for hybridization, and lack predicted secondary structure that may interfere with hybridization.

In one aspect, the oligonucleotides are synthesized at designated areas on the surface of a substrate, for example by using a light-directed chemical coupling procedure and an inkjet application apparatus, such as that described in WO95/251116 (Baldeschweiler el al.). The substrate may be paper, nylon or any other type of membrane, filter, chip, glass slide, or any other suitable solid support. In another aspect, a “gridded” array analogous to a dot or slot blot (HYBRIDOT apparatus, GIBCO/BRL) may be used to arrange and link cDNA fragments or oligonucleotides to the surface of a substrate using a vacuum system, thermal, UV, mechanical or chemical bonding procedures. In a most preferred embodiment, each of the different predefined regions is physically separated from each other of the different regions. In yet another aspect, an array may be produced by hand or by using available devices, materials, and machines (including BRINKMANN multichannel pipettors or robotic instruments). Such an array may contain 8, 24, 96, 384, 1536, or 6144 oligonucleotides, or any other multiple from 2 to 1,000,000 that lends itself to the efficient use of commercially available instrumentation. In one preferred embodiment the array includes at least 1,000 different oligonucleotides attached to surface of the solid support, and more preferably at least 10,000 different oligonucleotides. Oligonucleotides are preferably attached to the first surface of the solid support through a linker group. The oligonucleotide in the different predefined regions are at least 20% pure, more preferably are at least 50% pure, even more preferably at least 80% pure, and most preferably at least 90% pure.

In one embodiment, the array contains a planar, non-porous solid support having at least a first surface, and a plurality of different oligonucleotides attached to the first surface of the solid support at a density exceeding 400 different oligonucleotides per square cm, wherein each of the different oligonucleotides is attached to the surface of the solid support in a different predefined region, has a different determinable sequence, and is at least 6 nucleotides in length, with preferred lengths as discussed above, wherein at least one of the different oligonucletides is a PRO-C-MG.2, PRO-C-MG.12, PRO-C-MG.45, PRO-C-MG.64 or PRO-C-MG.72 sequence. In this embodiment each different oligonucleotides is from about 6 to about 20 nucleotides in length, more preferably at least 10 nucleotides in length, and most preferably at least 20 nucleotides in length. In a most preferred embodiment, each of the different predefined regions is physically separated from each other of the different regions. Oligonucleotides are preferably attached to the first surface of the solid support through a linker group. The oligonucleotide in the different predefined regions are at least 20% pure, more preferably are at least 50% pure, even more preferably at least 80% pure, and most preferably at least 90% pure.

Sample analysis using the microarrays can be conducted by extracting polynucleotides from a biological sample. The biological samples are obtained from any bodily fluid (blood, urine, saliva, phlegm, gastric juices, etc.), cultured cells, biopsies, or other tissue preparations. The polynucleotides extracted from the sample can be used to produce, as probes, nucleic acid sequences that are complementary to the nucleic acids on the microarray. If the microarray consists of cDNAs, antisense RNAs (aRNA) are appropriate probes. Therefore, in one aspect, mRNA is used to produce cDNA that, in turn and in the presence of fluorescent nucleotides, is used to produce fragment or oligonucleotide aRNA probes. These fluorescently-labeled probes are incubated with the microarray so that the probe sequences hybridize to the cDNA oligonucleotides of the microarray. In another aspect, nucleic acid sequences used as probes can include polynucleotides, fragments, and complementary or antisense sequences produced using restriction enzymes, PCR technologies, and OLIGOLABELING™ or TRANSPROBE™ kits (Pharmacia) well known in the area of hybridization technology. In an alternative microarray embodiment, oligonucleotides (preferably antisense molecules) are employed on the support and the target cDNA is the soluble binding component of the assay.

Incubation conditions are adjusted so that hybridization occurs with precise complementary matches or with various degrees of less complementarity. After removal of nonhybridized probes, a scanner is used to determine the levels and patterns of fluorescence. The scanned images are examined to determine degree of complementarity and the relative abundance of each oligonucleotide sequence on the microarray. A detection system may be used to measure the absence, presence, and amount of hybridization for all of the distinct sequences simultaneously. This data may be used for large-scale correlation studies or functional analysis of the sequences, mutations, variants, or polymorphisms among samples (Heller, R. A., et al., Proc. Natl. Acad. Sci. 94: 2150-55 (1997)).

For gene mapping, a gene or a cloned DNA fragment is hybridized to an ordered array of DNA fragments, and the identity of the DNA elements applied to the array is unambiguously established by the pixel or pattern of pixels of the array that are detected. In constructing physical maps of the genome, arrays of immobilized cloned DNA fragments are hybridized with other cloned DNA fragments to establish whether the cloned fragments in the probe mixture overlap and are therefore contiguous to the immobilized clones on the array. For example, Meier-Ewert et al., (J. Biotech. 35(2-3):191-203 (1994)) disclose such an application.

The arrays of immobilized DNA fragments may also be used for genetic diagnostics. For example, array containing multiple forms of a mutated gene or genes can be probed with a labeled mixture of a patient's DNA which will preferentially interact with only one of the immobilized versions of the gene. The detection of this interaction can provide a medical diagnosis. Arrays of immobilized DNA fragments can also be used in DNA probe diagnostics. For unambiguous genotyping or identifying a DNA- or RNA-containing sample as that of a human, the identity of the test sample can be established unambiguously by hybridizing the sample to an array containing DNA from different organisms, including human, wherein one or more PRO-C-MG.2, PRO-C-MG.12, PRO-C-MG.45, PRO-C-MG.64 or PRO-C-MG.72 genes sequences are included in the array. Other molecules of genetic interest, such as cDNAs and RNAs can be immobilized on the array or alternately used as the labeled probe mixture that is applied to the array.

In one embodiment, a potential antagonist includes a polypeptide or small molecule that binds to the fusions of immunoglobulin with PRO-C-MG.2, PRO-C-MG.12, PRO-C-MG.45, PRO-C-MG.64 or PRO-C-MG.72 polypeptide, and, in particular are antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist can be a closely related protein or peptide, for example, a mutated form of the PRO-C-MG.2, PRO-C-MG.12, PRO-C-MG.45, PRO-C-MG.64 or PRO-C-MG.72 polypeptide that recognizes a PRO-C-MG.2, PRO-C-MG.12, PRO-C-MG.45, PRO-C-MG.64 or PRO-C-MG.72 binding protein or substrate but imparts no effect, thereby competitively inhibiting the action of the PRO-C-MG.2, PRO-C-MG.12, PRO-C-MG.45, PRO-C-MG.64 or PRO-C-MG.72 polypeptide.

Another potential PRO-C-MG.2, PRO-C-MG.12, PRO-C-MG.45, PRO-C-MG.64 or PRO-C-MG.72 polypeptide antagonist is an antigene (antisense or sense) construct, as described herein, prepared using antisense technology, where, for example, the antisense molecule acts to reduces directly the translation of mRNA by hybridizing to targeted PRO-C-MG.2, PRO-C-MG.12, PRO-C-MG.45, PRO-C-MG.64 or PRO-C-MG.72 mRNA or the sense or antisense molecule reduces transcription of the mRNA by hybridizing to PRO-C-MG.2, PRO-C-MG.12, PRO-C-MG.45, PRO-C-MG.64 or PRO-C-MG.72 genomic DNA (typically through triple-helix formation), both means preventing or reducing protein translation of PRO-C-MG.2, PRO-C-MG.12, PRO-C-MG.45, PRO-C-MG.64 or PRO-C-MG.72. For example, the 5′ coding portion of the polynucleotide sequence, which encodes the mature PRO-C-MG.2, PRO-C-MG.12, PRO-C-MG.45, PRO-C-MG.64 or PRO-C-MG.72 polypeptides herein, is used to design an antisense RNA or DNA or PNA oligonucleotide of from about 5 to 60 base pairs in length. The antisense oligonucleotide hybridizes to the mRNA in vivo and blocks translation of the mRNA molecule into the PRO-C-MG.2, PRO-C-MG.12, PRO-C-MG.45, PRO-C-MG.64 or PRO-C-MG.72 polypeptide (antisense—Okano, Neurochem., 56:560 (1991); Oligodeoxynucleotides as Antisense Inhibitors of Gene Expression (CRC Press: Boca Raton, Fla., 1988). A PNA sense or antisense oligonucleotide is designed to be complementary to a region of the gene involved in transcription (triple helix—see Lee et al., Nucl. Acids Res., 6:3073 (1979); Cooney et al., Science, 241: 456 (1988); Dervan etal., Science, 251:1360 (1991)), thereby preventing transcription and the production of the PRO-C-MG.2, PRO-C-MG.12, PRO-C-MG.45, PRO-C-MG.64 or PROC-MG.72 polypeptide. The oligonucleotides described above can also be delivered to cells such that the antigene molecule can be expressed in vivo to inhibit production of the PRO-C-MG.2, PRO-C-MG.12, PRO-C-MG.45, PRO-C-MG.64 or PRO-C-MG.72 polypeptide. When antisense DNA is used, oligodeoxyribonucleotides derived from the translation-initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

As discussed herein, nucleic acid molecules in triple-helix formation used to inhibit transcription can be single-stranded and composed of deoxynucleotides. Such molecules can have backbone bonds not naturally found in DNA or RNA. A preferred form are PNAs. Such molecules that form a triplex with PRO-C-MG.2, PRO-C-MG.12, PRO-C-MG.45, PRO-C-MG.64 or PRO-C-MG.72 gene can also act as agonists to up-modulate PRO-C-MG.2, PRO-C-MG.12, PRO-C-MG.45, PRO-C-MG.64 or PRO-C-MG.72 transcription when appropriately targeted as discussed herein.

Potential antagonists include small molecules that bind to the active site, the protein binding site, or other relevant binding site (e.g., co-factor binding site, substrate binding site) of the PRO-C-MG.2, PRO-C-MG.12, PRO-C-MG.45, PRO-C-MG.64 or PRO-C-MG.72 polypeptide, thereby blocking the normal biological activity of the PRO-C-MG.2, PRO-C-MG.12, PRO-C-MG.45, PRO-C-MG.64 or PRO-C-MG.72 polypeptide. Examples of small molecules include, but are not limited to, small peptides or peptide-like molecules, preferably soluble peptides, and synthetic non-peptidyl organic or inorganic compounds.

These small molecules can be identified by any one or more of the screening assays discussed herein and/or by any other screening techniques well known for those skilled in the art.

For example, the PRO-C-MG.2, PRO-C-MG.12, PRO-C-MG.45, PRO-C-MG.64 or PRO-C-MG.72 agonist can be screened for the ability to stimulate or reduce the proliferation of or tube formation of endotlielial cells as described herein. In brief, in the proliferation assay, human umbilical vein endothelial cells are obtained and cultured in 96-well flat-bottomed culture plates (Costar, Cambridge, Mass.) and supplemented with a reaction mixture appropriate for facilitating proliferation of the cells. The compound to be screened is added and, after incubation at 37° C., cultures are pulsed with 3-H-thymidine and harvested onto glass fiber filters (phD; Cambridge Technology, Watertown, Mass.). Mean 3-H-thymidine incorporation (cpm) of triplicate cultures is determined using a liquid scintillation counter (Beckman Instruments, Irvine, Calif.). Significant 3-(H)thymidine incorporation indicates stimulation of endothelial cell proliferation. To assay for antagonists, the assays described herein can be performed. For example, in the above assay, a compound to be screened is added and its ability to inhibit 3-(H)thymidine incorporation is determined.

The compositions useful in the treatment of disorders and conditions provided herein include, without limitation, antibodies, small organic and inorganic molecules, peptides, phosphopeptides, antisense and ribozyme molecules, triple-helix molecules, etc., that inhibit the expression and/or activity of the target gene product.

The PRO-C-MG.2, PRO-C-MG.12, PRO-C-MG.45, PRO-C-MG.64 or PRO-C-MG.72 polypeptides and nucleic acid molecules of the present invention are particularly useful for detecting, monitoring, analyzing, or identifying, as described herein, the occurrence or progression of angiogenesis or vasculogenesis, as can occur, for example, in blood vessel repair and formation after trauma, such as after surgery, or during disorders or conditions such as cancer, tumor growth, or neovascularization. Angiogenesis, in which endothelial cells differentiate into endothelial cell tube-like structures that are precursor structures to vessel formation, is an important component of a variety of diseases and disorders including trauma, tumor growth and metastasis, rheumatoid arthritis, psoriasis, atherosclerosis, diabetic retinopathy, retrolental fibroplasia, neovascular glaucoma, age-related macular degeneration, hemangiomas, immune rejection of transplanted comeal tissue and other tissues, and chronic inflammation. By reducing vessel formation, the invention reduces the vasculature supporting a tumor, inhibiting tumor size or growth and reducing the tumor burden of the mammal. Conversely, by enhancing vessel formation, the invention increases or restores the vasculature supporting damaged tissue. Accordingly, the present invention provides means to detect, monitor, analyze, identify, or treat the occurrence or progression of angiogenesis or vasculogenesis in these and other related conditions, and to identify drugs, e.g., antisense, small molecule, antibody, useful to treat these and other related conditions.

Various assays can be used to test the polypeptide herein for angiogenic activity. Such assays include those provided in the Examples below.

Assays for tissue generation activity include, without limitation, those described in WO 95/16035 (bone, cartilage, tendon); WO 95/05846 (nerve, neuronal), and WO 91/07491 (skin, endothelium).

Assays for wound-healing activity include, for example, those described in Winter, Epidermal Wound Healing, Maibach, H I and Rovee, D T, eds. (Year Book Medical Publishers, Inc., Chicago), pp. 71-112, as modified by the article of Eagistein and Mertz, J. Invest. Dermatol., 71: 382-384 (1978).

Cell-Based Assays

Cell-based assays and animal models for angiogenic disorders, such as tumors, can be used to verify the findings of an angiogenic or angiostatic assay herein, and further to understand the relationship between the genes identified herein and the development and pathogenesis of undesirable angiogenic cell growth. The role of gene products identified herein in the development and pathology of desirable or undesirable angiogenic cell growth, e.g., endothelial cells, tumor cells, can be tested by using cells or cells lines that have been identified as being stimulated or inhibited by the PRO-C-MG.2, PRO-C-MG.12, PRO-C-MG.45, PRO-C-MG.64 or PRO-C-MG.72 polypeptide, or its agonists or antagonists, herein. Such cells include, for example, those set forth in the Examples below.

In a different approach, cells of a cell type known to be involved in a particular angiogenic activity or disorder are transfected with the cDNAs herein, and the ability of these cDNAs to induce excessive growth or inhibit growth is analyzed. If the angiogenic disorder is cancer, suitable tumor cells include, for example, stable tumor cells lines such as the B104-1-1 cell line (stable NIH-3T3 cell line transfected with the neu protooncogene) and ras-transfected NIH-3T3 cells, which can be transfected with the desired gene and monitored for tumorigenic growth. Such transfected cell lines can then be used to test the ability of poly- or monoclonal antibodies or antibody compositions to inhibit tumorigenic cell growth by exerting cytostatic or cytotoxic activity on the growth of the transformed cells, or by mediating antibody-dependent cellular cytotoxicity (ADCC). Cells transfected with the coding sequences of the genes identified herein can further be used to identify drug candidates for the treatment of angiogenic disorders such as cancer.

In another assay, human umbilical cord endothelial cells (HUVECS) undergoing tube formation in three-dimensional gels in the presence of growth factors, mimic the angiogenic environment of endothelial cells in vivo, providing a well-accepted system for angiogenisis and vasculogenesis, both in normal and neoplastic conditions (see for example Davis, et al. Exp. Cell Res. 1996 224:39-51 (1996) and the Examples herein). For example, in one tube formation assay, endothelial cells are suspended in a three-dimensional collagen lattice of type I collagen and undergo rapid morphogenesis. Within 4 hours numerous vacuoles are observed in the majority of endothelial cells. At 24 hours the formation of tube-like structures can be observed. And at 48 hours an interconnected network of tube-like structures is observed. In this and other tube formation assays, inhibitors of protein synthesis (cycloheximide) and mRNA synthesis (actinomycin D) completely block tube-formation. The three dimensional gel is pre-requisite for the differentiation and fusion of endothelial cells into tubes; HUVECS grown on the surface of gelatin or on plastic do not undergo tube-formation. HUVECS can be grown under various conditions, inductive or non-inductive to tube formation, either on gelatin or collagen film (non-inductive) or in collagen gels (inductive), with or without the addition of growth factors to simulate normal angiogenic- or tumor-derived factors. HUVEC cells can be transfected with the cDNAs (or their antisense) herein, and the ability of these nucleic acids to induce excessive growth or tube formation or inhibit growth or tube formation is analyzed. HUVEC cells expressing coding sequences of the genes identified herein can further be used to identify drug candidates. PCR can be used detect the expression of a PRO-C-MG.2, PRO-C-MG.12, PRO-C-MG.45, PRO-C-MG.64 or PRO-C-MG.72 mRNA in the endothelial cells cultured in 3D gels, as well as in any other cell or organism. In addition, primary cultures derived from tumors in transgenic animals (as described above) can be used in the cell-based assays herein, although stable cell lines are preferred. Techniques to derive continuous cell lines from transgenic animals are well known in the art. See, e.g., Small et al., Mol. Cell. Biol., 5: 642-648 (1985).

For cancer, a variety of well-known animal models can be used to further understand the role of the genes identified herein in the development and pathogenesis of tumors, and to test the efficacy of candidate therapeutic agents, including antibodies and other antagonists of the native PRO-C-MG.2, PRO-C-MG.12, PRO-C-MG.45, PRO-C-MG.64 or PRO-C-MG.72 polypeptides, such as small-molecule antagonists. The in vivo nature of such models makes them particularly predictive of responses in human patients. Animal models of tumors and cancers (e.g., breast cancer, colon cancer, prostate cancer, lung cancer, etc.) include both non-recombinant and recombinant (transgenic) animals. Non-recombinant animal models include, for example, rodent, e.g., murine models. Such models can be generated by introducing tumor cells into syngeneic mice using standard techniques, e.g., subcutaneous injection, tail vein injection, spleen implantation, intraperitoneal implantation, implantation under the renal capsule, or orthopin implantation, e.g., colon cancer cells implanted in colonic tissue. See, e.g., PCT publication No. WO 97/33551, published Sep. 18, 1997. Probably the most often used animal species in oncological studies are immunodeficient mice and, in particular, nude mice. The observation that the nude mouse with thymic hypo/aplasia could successfully act as a host for human tumor xenografts has lead to its widespread use for this purpose. The autosomal recessive nu gene has been introduced into a very large number of distinct congenic strains of nude mouse, including, for example, ASW, A/He, AKR, BALB/c, B10.LP, C17, C3H, C57BL, C57, CBA, DBA, DDD, I/st, NC, NFR, NFS, NFS/N, NZB, NZC, NZW, P, RIII, and SJL. In addition, a wide variety of other animals with inherited immunological defects other than the nude mouse have been bred and used as recipients of tumor xenografts. For further details see, e.g., The Nude Mouse in Oncology Research, E. Boven and B. Winograd, eds. (CRC Press, Inc., 1991).

The cells introduced into such animals can be derived from known tumor/cancer cell lines, such as any of the above-listed tumor cell lines, and, for example, the B104-1-1 cell line (stable NIH-3T3 cell line transfected with the neu protooncogene); ras-transfected NIH-3T3 cells; Caco-2 (ATCC HTB-37); or a moderately well-differentiated grade 11 human colon adenocarcinoma cell line, HT-29 (ATCC HTB-38); or from tumors and cancers. Samples of tumor or cancer cells can be obtained from patients undergoing surgery, using standard conditions involving freezing and storing in liquid nitrogen. Karmali et al., Br. J. Cancer, 48: 689-696 (1983).

Tumor cells can be introduced into animals such as nude mice by a variety of procedures. The subcutaneous (s.c.) space in mice is very suitable for tumor implantation. Tumors can be transplanted s.c. as solid blocks, as needle biopsies by use of a trochar, or as cell suspensions. For solid-block or trochar implantation, tumor tissue fragments of suitable size are introduced into the s.c. space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue.

Animal models of breast cancer can be generated, for example, by implanting rat neuroblastoma cells (from which the neu oncogene was initially isolated), or neu-transformed NIH-3T3 cells into nude mice, essentially as described by Drebin et al. Proc. Nat. Acad. Sci. USA, 83: 9129-9133 (1986).

Similarly, animal models of colon cancer can be generated by passaging colon cancer cells in animals, e.g., nude mice, leading to the appearance of tumors in these animals. An orthotopic transplant model of human colon cancer in nude mice has been described, for example, by Wang et al., Cancer Research, 54: 4726-4728 (1994) and Too et al., Cancer Research, 55: 681-684 (1995). This model is based on the so-called “METAMOUSE”™ sold by AntiCancer, Inc., (San Diego, Calif.).

Tumors that arise in animals can be removed and cultured in vitro. Cells from the in vitro cultures can then be passaged to animals. Such tumors can serve as targets for further testing or drug screening. Alternatively, the tumors resulting from the passage can be isolated and RNA from pre-passage cells and cells isolated after one or more rounds of passage analyzed for differential expression of genes of interest. Such passaging techniques can be performed with any known tumor or cancer cell lines.

For example, Meth A, CMS4, CMS5, CMS21, and WEHI-164 are chemically induced fibrosarcomas of BALB/c female mice (DeLeo et al., J. Exp. Med., 146: 720 (1977)), which provide a highly controllable model system for studying the anti-tumor activities of various agents. Palladino et al., J. Immunol., 138: 4023-4032 (1987). Briefly, tumor cells are propagated in vitro in cell culture. Prior to injection into the animals, the cell lines are washed and suspended in buffer, at a cell density of about 10×106 to 10×107 cells/ml. The animals are then infected subcutaneously with 10 to 100 μl of the cell suspension, allowing one to three weeks for a tumor to appear.

In addition, the Lewis lung (3LL) carcinoma of mice, which is one of the most thoroughly studied experimental tumors, can be used as an investigational tumor model. Efficacy in this tumor model has been correlated with beneficial effects in the treatment of human patients diagnosed with small-cell carcinoma of the lung (SCCL). This tumor can be introduced in normal mice upon injection of tumor fragments from an affected mouse or of cells maintained in culture. Zupi et al., Br. J. Cancer, 41: suppl. 4, 30 (1980). Evidence indicates that tumors can be started from injection of even a single cell and that a very high proportion of infected tumor cells survive. For further information about this tumor model see, Zacharski, Haemostasis 16: 300-320 (1986).

One way of evaluating the efficacy of a test compound in an animal model with an implanted tumor is to measure the size of the tumor before and after treatment. Traditionally, the size of implanted tumors has been measured with a slide caliper in two or three dimensions. The measure limited to two dimensions does not accurately reflect the size of the tumor; therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6^{th} Int. Workshop on Immune-Deficient Animals, Wu and Sheng eds. (Basel, 1989), p. 301. It is noted, however, that necrosis and inflammatory responses following treatment may actually result in an increase in tumor size, at least initially. Therefore, these changes need to be carefully monitored, by a combination of a morphometric method and flow cytometric analysis.

Further, recombinant (transgenic) animal models can be engineered by introducing the coding portion of the PRO-C-MG.2, PRO-C-MG.12, PRO-C-MG.45, PRO-C-MG.64 or PRO-C-MG.72 genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (U.S. Pat. No. 4,873,191); retrovirus-mediated gene transfer into germ lines (e.g., Van der Putten et al., Proc. Natl. Acad. Sci. USA, 82: 6148-615 (1985)); gene targeting in embryonic stem cells (Thompson et al., Cell, 56: 313-321 (1989)); electroporation of embryos (Lo, Mol. Cell. Biol., 3: 1803-1814 (1983)); and sperm-mediated gene transfer. Lavitrano et al., Cell, 57: 717-73 (1989). For a review, see for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89: 6232-636 (1992). The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock-out” animals can be constructed that have a defective or altered gene encoding a PRO-C-MG.2, PRO-C-MG.12, PRO-C-MG.45, PRO-C-MG.64 or PRO-C-MG.72 polypeptide identified herein, as a result of homologous recombination between the endogenous gene encoding the PRO-C-MG.2, PRO-C-MG.12, PRO-C-MG.45, PRO-C-MG.64 or PRO-C-MG.72 polypeptide and altered genomic DNA encoding the same polypeptide introduced into an embryonic cell of the animal. For example, cDNA encoding a particular PRO-C-MG.2, PRO-C-MG.12, PRO-C-MG.45, PRO-C-MG.64 or PRO-C-MG.72 polypeptide can be used to clone genomic DNA encoding that polypeptide in accordance with established techniques. A portion of the genomic DNA encoding a particular PRO-C-MG.2, PRO-C-MG.12, PRO-C-MG.45, PRO-C-MG.64 or PRO-C-MG.72 polypeptide can be deleted or replaced with another gene, such as a gene encoding a selectable marker that can be used to monitor integration. Typically, several kilobases of unaltered flanking DNA (both at the 5′ and 3′ ends) are included in the vector. See, e.g., Thomas and Capecchi, Cell, 51: 503 (1987) for a description of homologous recombination vectors. The vector is introduced into an embryonic stem cell line (e.g., by electroporation) and cells in which the introduced DNA has homologously recombined with the endogenous DNA are selected. See, e.g., Li et al., Cell, 69: 915 (1992). The selected cells are then injected into a blastocyst of an animal (e.g., a mouse or rat) to form aggregation chimeras. See, e.g., Bradley, in Teratocarcinomas and Embryonic Stem Cells: A Practical Approach, E. J. Robertson, ed. (IRL: Oxford, 1987), pp. 113-152. A chimeric embryo can then be implanted into a suitable pseudopregnant female foster animal and the embryo brought to term to create a “knock-out” animal. Progeny harboring the homologously recombined DNA in their germ cells can be identified by standard techniques and used to breed animals in which all cells of the animal contain the homologously recombined DNA. Knock-out animals can also be generated, as is well knows in the art, by administering an antisense molecule of the invention. Animals comprising such antisense molecules are specifically contemplanted as an embodiment of the invention. Knockout animals can be characterized, for instance, by their ability to defend against certain pathological conditions and by their development of pathological conditions due to absence (knock-out) of the PRO-C-MG.2, PRO-C-MG.12, PRO-C-MG.45, PRO-C-MG.64 or PRO-C-MG.72 polypeptide.

The efficacy of antibodies specifically binding the PRO-C-MG.2, PRO-C-MG.12, PRO-C-MG.45, PRO-C-MG.64 or PRO-C-MG.72 polypeptides identified herein, and other drug candidates, can be tested also in the treatment of spontaneous animal tumors. A suitable target for such studies is the feline oral squamous cell carcinoma (SCC). Feline oral SCC is a highly invasive, malignant tumor that is the most common oral malignancy of cats, accounting for over 60% of the oral tumors reported in this species. It rarely metastasizes to distant sites, although this low incidence of metastasis may merely be a reflection of the short survival times for cats with this tumor. These tumors are usually not amenable to surgery, primarily because of the anatomy of the feline oral cavity. At present, there is no effective treatment for this tumor. Prior to entry into the study, each cat undergoes complete clinical examination and biopsy, and is scanned by computed tomography (CT). Cats diagnosed with sublingual oral squamous cell tumors are excluded from the study. The tongue can become paralyzed as a result of such tumor, and even if the treatment kills the tumor, the animals may not be able to feed themselves. Each cat is treated repeatedly, over a longer period of time. Photographs of the tumors will be taken daily during the treatment period, and at each subsequent recheck. After treatment, each cat undergoes another CT scan. CT scans and thoracic radiograms are evaluated every 8 weeks thereafter. The data are evaluated for differences in survival, response, and toxicity as compared to control groups. Positive response may require evidence of tumor regression, preferably with improvement of quality of life and/or increased life span.

In addition, other spontaneous animal tumors, such as fibrosarcoma, adenocarcinoma, lymphoma, chondroma, or leiomyosarcoma of dogs, cats, and baboons can also be tested. Of these, mammary adenocarcinoma in dogs and cats is a preferred model as its appearance and behavior are very similar to those in humans. However, the use of this model is limited by the rare occurrence of this type of tumor in animals. Other in vitro and in vivo angiogenic tests known in the art are also suitable herein.

H. Anti-PRO-C-MG.2, PRO-C-MG.12, PRO-C-MG.45, PRO-C-MG.64 or PRO-C-MG.72 Antibodies

The present invention further provides anti-PRO-C-MG.2, PRO-C-MG.12, PRO-C-MG.45, PRO-C-MG.64 or PRO-C-MG.72 antibodies. Exemplary antibodies include polyclonal, monoclonal, humanized, bispecific, and heteroconjugate antibodies.

1. Polyclonal Antibodies

The anti-PRO-C-MG.2, PRO-C-MG.12, PRO-C-MG.45, PRO-C-MG.64 or PRO-C-MG.72 antibodies can comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent can include the PRO-C-MG.2, PRO-C-MG.12, PRO-C-MG.45, PRO-C-MG.64 or PRO-C-MG.72 polypeptide or a fusion protein thereof. It can be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which can be employed include Freund's complete adjuvant and MPL-TDM adjuvant (monophosphoryl Lipid A, synthetic trehalose dicorynomycolate). The immunization protocol can be selected by one skilled in the art without undue experimentation.

2. Monoclonal Antibodies

The anti-PRO-C-MG.2, PRO-C-MG.12, PRO-C-MG.45, PRO-C-MG.64 or PRO-C-MG.72 antibodies can, alternatively, be monoclonal antibodies. Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the PRO-C-MG.2, PRO-C-MG.12, PRO-C-MG.45, PRO-C-MG.64 or PRO-C-MG.72 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against PRO-C-MG.2, PRO-C-MG.12, PRO-C-MG.45, PRO-C-MG.64 or PRO-C-MG.72. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No.4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-PRO-C-MG.2, PRO-C-MG.12, PRO-C-MG.45, PRO-C-MG.64 or PRO-C-MG.72 antibodies of the invention can further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human imniunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

4. Bispecific Antibodies

Bispecific antibodies are monoclonal, preferably human or humanized, antibodies that have binding specificities for at least two different antigens. In the present case. one of the binding specificities is for the PRO-C-MG.2, PRO-C-MG.12, PRO-C-MG.45, PRO-C-MG.64 or PRO-C-MG.72, the other one is for any other antigen, and preferably for a cell-surface protein or receptor or receptor subunit.

Methods for making bispecific antibodies are known in the art. Traditionally, the recombinant production of bispecific antibodies is based on tile co-expression of two immunoglobulin heavy-chain/light-chiain pairs. where the two heavy chains have different specificities [Milstein and Cuello, Nature. 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber el al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes on a given PRO-C-MG.2, PRO-C-MG.12, PRO-C-MG.45, PRO-C-MG.64 or PRO-C-MG.72 polypeptide herein. Alternatively, an anti-PRO-C-MG.2, PRO-C-MG.12, PRO-C-MG.45, PRO-C-MG.64 or PRO-C-MG.72 polypeptide arm can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular PRO-C-MG.2, PRO-C-MG.12, PRO-C-MG.45, PRO-C-MG.64 or PRO-C-MG.72 polypeptide. Bispecific antibodies can also be used to localize cytotoxic agents to cells which express a particular PRO-C-MG.2, PRO-C-MG.12, PRO-C-MG.45, PRO-C-MG.64 or PRO-C-MG.72 polypeptide. These antibodies possess a PRO-C-MG.2, PRO-C-MG.12, PRO-C-MG.45, PRO-C-MG.64 or PRO-C-MG.72-binding arm and an arm which binds a cytotoxic agent or a radionuclide chelator, such as EOTUBE, DPTA, DOTA, or TETA. Another bispecific antibody of interest binds the PRO-C-MG.2, PRO-C-MG.12, PRO-C-MG.45, PRO-C-MG.64 or PRO-C-MG.72 polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry. including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed. for example. in U.S. Pat. No. 4.676.980.

6. Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelon in, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA. 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

9. Pharmaceutical Compositions of Antibodies

Antibodies specifically binding a PRO-C-MG.2, PROC-MG.12, PRO-C-MG.45, PRO-C-MG.64 or PRO-C-MG.72 polypeptide identified herein, as well as other molecules identified by the screening assays disclosed herein, can be administered for the treatment of various disorders in the form of pharmaceutical compositions.

If the PRO-C-MG.2, PRO-C-MG.12, PRO-C-MG.45, PRO-C-MG.64 or PRO-C-MG.72 polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization can be achieved by modifying sulfhydryl residues, lyophilizing from acidic solutions, controlling moisture content, using appropriate additives, and developing specific polymer matrix compositions.

I. Uses for Anti-PRO-C-MG.2, PRO-C-MG.12, PRO-C-MG.45, PRO-C-MG.64 or PRO-C-MG.72 Antibodies

The anti-PRO-C-MG.2, PRO-C-MG.12, PRO-C-MG.45, PRO-C-MG.64 or PRO-C-MG.72 antibodies of the invention have various utilities. For example, anti-PRO-C-MG.2, PRO-C-MG.12, PRO-C-MG.45, PRO-C-MG.64 or PRO-C-MG.72 antibodies can be used in diagnostic assays for PRO-C-MG.2, PRO-C-MG.12, PRO-C-MG.45, PRO-C-MG.64 or PRO-C-MG.72, e.g., detecting its expression in specific cells, tissues, or serum. Various diagnostic assay techniques known in the art can be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety can be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety can be employed, including those methods described by Hunter et al., Nature, 144:945 (1962); David et al., Biochemistry, 13:1014 (1974); Pain et al., J. Immunol. Meth., 40:219 (1981); and Nygren, J. Histochem and Cytochem., 30:407 (1982).

Anti-PRO-C-MG.2, PRO-C-MG.12, PRO-C-MG.45, PRO-C-MG.64 or PRO-C-MG.72 antibodies also are useful for the affinity purification of PRO-C-MG.2, PRO-C-MG.12, PRO-C-MG.45, PRO-C-MG.64 or PRO-C-MG.72 from recombinant cell culture or natural sources. In this process, the antibodies against PRO-C-MG.2, PRO-C-MG.12, PRO-C-MG.45, PRO-C-MG.64 or PRO-C-MG.72 are immobilized on a suitable support, such a Sephadex resin or filter paper, using methods well known in the art. The immobilized antibody then is contacted with a sample containing tie PRO-C-MG.2, PRO-C-MG.12, PRO-C-MG.45, PRO-C-MG.64 or PRO-C-MG.72 to be purified, and thereafter the support is washed with a suitable solvent that will remove substantially all the material in the sample except the PRO-C-MG.2, PRO-C-MG.12, PRO-C-MG.45, PRO-C-MG.64 or PRO-C-MG.72, which is bound to the immobilized antibody. Finally, the support is washed with another suitable solvent that will release the PRO-C-MG.2, PRO-C-MG.12, PRO-C-MG.45, PRO-C-MG.64 or PRO-C-MG.72 from the antibody.

J. Use of Gene as Diagnostic

This invention is also related to the use of the gene encoding the PRO-C-MG.2, PRO-C-MG.12, PRO-C-MG.45, PRO-C-MG.64 or PRO-C-MG.72 polypeptide as a diagnostic. Detection of a mutated form of the PRO-C-MG.2, PRO-C-MG.12, PRO-C-MG.45, PRO-C-MG.64 or PRO-C-MG.72 polypeptide will allow a diagnosis of an angiogenic disease or a susceptibility to a angiogenic disease, such as a tumor, since mutations in the PRO-C-MG.2, PRO-C-MG.12, PRO-C-MG.45, PRO-C-MG.64 or PRO-C-MG.72 polypeptide may cause tumors.

Individuals carrying mutations in the genes encoding a human PRO-C-MG.2, PRO-C-MG.12, PRO-C-MG.45, PRO-C-MG.64 or PRO-C-MG.72 polypeptide may be detected at the DNA level by a variety of techniques. Nucleic acids for diagnosis may be obtained from a patient's cells, such as from blood, urine, saliva, tissue biopsy, and autopsy material. The genomic DNA may be used directly for detection or may be amplified enzymatically by using PCR (Saiki et al., Nature, 324: 163-166 (1986)) prior to analysis. RNA or cDNA may also be used for the same purpose. As an example, PCR primers complementary to the nucleic acid encoding the PRO-C-MG.2, PRO-C-MG.12, PRO-C-MG.45, PRO-C-MG.64 or PRO-C-MG.72 polypeptide can be used to identify and analyze PRO-C-MG.2, PRO-C-MG.12, PRO-C-MG.45, PRO-C-MG.64 or PRO-C-MG.72 polypeptide mutations. For example, deletions and insertions can be detected by a change in size of the amplified product in comparison to the normal genotype. Point mutations can be identified by hybridizing amplified DNA to radiolabeled RNA encoding the PRO-C-MG.2, PRO-C-MG.12, PRO-C-MG.45, PRO-C-MG.64 or PRO-C-MG.72 polypeptide, or alternatively, radiolabeled antisense DNA sequences encoding the PRO-C-MG.2, PRO-C-MG.12, PRO-C-MG.45, PRO-C-MG.64 or PRO-C-MG.72 polypeptide. Perfectly matched sequences can be distinguished from mismatched duplexes by RNase A digestion or by differences in melting temperatures.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamidine gradient gels in which the mobilities of different DNA fragments are retarded in the gel at different positions according to their specific melting or partial melting temperatures. See, e.g., Myers et al., Science, 230: 1242 (1985).

Sequence changes at specific locations may also be revealed by nuclease protection assays, such as RNase and S1 protection or the chemical cleavage method, for example, Cotton et al, Proc. Natl. Acad. Sci. USA, 85: 4397-4401 (1985).

Thus, the detection of a specific DNA sequence may be achieved by methods such as hybridization, RNase protection, chemical cleavage, direct DNA sequencing, or the use of restriction enzymes, e.g., restriction fragment length polymorphisms (RFLP), and Southern blotting of genomic DNA.

K. Use to Detect PRO-C-MG.2, PRO-C-MG.12, PRO-C-MG.45, PRO-C-MG.64 or PRO-C-MG.72 Polypentide Levels

In addition to more conventional gel-electrophoresis and DNA sequencing, mutations can also be detected by in situ analysis. Expression of nucleic acid encoding the PRO-C-MG.2, PRO-C-MG.12, PRO-C-MG.45, PRO-C-MG.64 or PRO-C-MG.72 polypeptide can be linked to vascular disease or neovascularization associated with tumor formation. A sample, e.g. biopsy, of the suspected tissue or tumor mass can be contacted with an anti-PRO-C-MG.2, PRO-C-MG.12, PRO-C-MG.45, PRO-C-MG.64 or PRO-C-MG.72 polypeptide antibody to diagnose vascular disease or neovascularization associated with tumor formation, since an altered level of this PRO-C-MG.2, PRO-C-MG.12, PRO-C-MG.45, PRO-C-MG.64 or PRO-C-MG.72 polypeptide can be indicative of such disorders. A competition assay can be employed wherein antibodies specific to the PRO-C-MG.2, PRO-C-MG.12, PRO-C-MG.45, PRO-C-MG.64 or PRO-C-MG.72 polypeptide are attached to a solid support and the labeled PRO-C-MG.2, PRO-C-MG.12, PRO-C-MG.45, PRO-C-MG.64 or PRO-C-MG.72 polypeptide and an appropriately processed sample derived from the subject are passed over the solid support, wherein the amount of label detected attached to the solid support is correlated to a quantity of PRO-C-MG.2, PRO-C-MG.12, PRO-C-MG.45, PRO-C-MG.64 or PRO-C-MG.72 polypeptide in the sample.

Since expression of PRO-C-MG.2, PRO-C-MG.12, PRO-C-MG.45, PRO-C-MG.64 or PRO-C-MG.72 mRNA is correlated with angiogenesis as disclosed herein, in anther embodiment a PRO-C-MG.2, PRO-C-MG.12, PRO-C-MG.45, PRO-C-MG.64 or PRO-C-MG.72 specific nucleic acid of the invention can be used in an RNA detection or quantification method, such as in situ hybridization or PCR amplification, to diagnose or detect vascular disease or neovascularization associated with tumor formation.

L. Types of Angiogenic Disorders to be Treated

The PRO-C-MG.2, PRO-C-MG.12, PRO-C-MG.45, PRO-C-MG.64 or PRO-C-MG.72 polypeptides, or agonists or antagonists thereto, that have activity in the cardiovascular, angiogenic, and endothelial assays described herein, are likely to have therapeutic uses in a variety of angiogenic disorders, including systemic disorders that affect vessels, such as diabetes mellitus. Their therapeutic utility could include diseases of the arteries, capillaries, veins, and/or lymphatics. The compounds of the invention thus have use in treatment of diseases or disorders characterized by undesirable excessive neovascularization. Vascular or angiogenic dysfunction further includes diseases of the vessels themselves, such as of the arteries, capillaries, veins, and/or lymphatics. This would include indications that stimulate angiogenesis, cardiovascularization, and/or neovascularization, and those that inhibit angiogenesis, cardiovascularization, and/or neovascularization. Such disorders include, for example, arterial disease, such as atherosclerosis, hypertension, inflammatory vasculitides, Reynaud's disease and Reynaud's phenomenon, aneurysms, and arterial restenosis; venous and lymphatic disorders such as thrombophlebitis, lymphangitis, and lymphedema; and other vascular disorders such as peripheral vascular disease, cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma, tumor angiogenesis, trauma such as wounds, burns, and other injured tissue, implant fixation, scarring, ischemia reperfusion injury, rheumatoid arthritis, psoriasis, retinopathy, retrolental fibroplasia, neovascular glaucoma, age-related macular degeneration, thyroid hyperplasias, Grave's disease, tissue transplantation, chronic inflammation, lung inflammation, obesity, cerebrovascular disease, renal diseases such as acute renal failure, and osteoporosis. This would also include angina, myocardial infarctions such as acute myocardial infarctions and heart failure such as congestive heart failure.

The PRO-C-MG.2, PRO-C-MG.12, PRO-C-MG.45, PRO-C-MG.64 or PRO-C-MG.72 polypeptides or agonists or antagonists thereto may also be employed to stimulate wound healing or tissue regeneration and associated therapies concerned with re-growth of tissue, such as connective tissue, skin, bone, cartilage, muscle, lung, or kidney, to promote angiogenesis, and to proliferate the growth of vascular smooth muscle and endothelial cell production, and improving allograft and xenograft success. The increase in angiogenes is mediated by the PRO-C-MG.2, PRO-C-MG.12, PRO-C-MG.45, PRO-C-MG.64 or PRO-C-MG.72 polypeptide or antagonist would be beneficial to ischemic tissues and to collateral coronary development in the heart subsequent to coronary stenosis. Antagonists are used to inhibit the action of such polypeptides, for example, to limit the production of excess connective tissue during wound healing or pulmonary fibrosis if the PROC-MG.2, PRO-C-MG.12, PRO-C-MG.45, PRO-C-MG.64 or PRO-C-MG.72 polypeptide promotes such production. This would include treatment of acute myocardial infarction and heart failure, other trauma of the vasculature, and muscle wasting disease.

Moreover, the present invention concerns the treatment of cardiac hypertrophy, regardless of the underlying cause, by administering a therapeutically effective dose of the PRO-C-MG.2, PRO-C-MG.12, PRO-C-MG.45, PRO-C-MG.64 or PRO-C-MG.72 polypeptide, or agonist or antagonist thereto. If the objective is the treatment of human patients, the PRO-C-MG.2, PRO-C-MG.12, PRO-C-MG.45, PRO-C-MG.64 or PRO-C-MG.72 polypeptide preferably is recombinant human PRO-C-MG.2, PRO-C-MG.12, PROC-MG.45, PRO-C-MG.64 or PRO-C-MG.72 polypeptide (rhPRO-C-MG.2, PRO-C-MG.12, PRO-C-MG.45, PRO-C-MG.64 or PRO-C-MG.72 or rhPRO-C-MG.2, PRO-C-MG.12, PRO-C-MG.45, PRO-C-MG.64 or PRO-C-MG.72 polypeptide). The treatment for cardiac hypertrophy can be performed at any of its various stages, which may result from a variety of diverse pathologic conditions, including myocardial infarction, hypertension, hypertrophic cardiomyopathy, and valvular regurgitation. The treatment extends to all stages of the progression of cardiac hypertrophy, with or without structural damage of the heart muscle, regardless of the underlying cardiac disorder.

The decision of whether to use the molecule itself or an agonist thereof for any particular indication, as opposed to an antagonist to the molecule, would depend mainly on whether the molecule herein promotes cardiovascularization, genesis of endothelial cells, or angiogenesis or inhibits these conditions. For example, if the molecule promotes angiogenesis, an antagonist thereof would be useful for treatment of disorders where it is desired to limit or prevent angiogenesis. Examples of such disorders include vascular tumors such as haemangioma, tumor angiogenesis, neovascularization in the retina, choroid, or cornea, associated with diabetic retinopathy or premature infant retinopathy or macular degeneration and proliferative vitreoretinopathy, rheumatoid arthritis, Crohn's disease, atherosclerosis, ovarian hyperstimulation, psoriasis, endometriosis associated with neovascularization, restenosis subsequent to balloon angioplasty, scar tissue overproduction, for example, that seen in a keloid that forms after surgery, fibrosis after myocardial infarction, or fibrotic lesions associated with pulmonary fibrosis.

Eexcessive endometrial angiogenesis has been proposed as an important mechanism in the pathogenesis of endometriosis. The endometrium of women with endometriosis has an increased capacity to proliferate, implant and grow in the peritoneal cavity. The endometrium of patients with endometriosis shows enhanced endothelial cell proliferation. Cell adhesion molecule integrin alphavbeta3 is expressed in more blood vessels in the endometrium of women with endometriosis when compared with normal women. Taken together, these results provide evidence for increased endometrial angiogenesis in women with endometriosis when compared with normal subjects (Healy et al. Hum. Reprod. Update 4(5):736-40 (1998)). Endometriosis is one of the family of angiogenic diseases, as discussed herein. Inhibition of angiogenesis as taught herein will provide benefit in treating such a disease.

If, however, the molecule inhibits angiogenesis, it would be expected to be used directly for treatment of the above conditions.

On the other hand, if the molecule stimulates angiogenesis it would be used itself (or an agonist thereof) for indications where angiogenesis is desired such as peripheral vascular disease, hypertension, inflammatory vasculitides, Reynaud's disease and Reynaud's phenomenon, aneurysms, arterial restenosis, thrombophlebitis, lymphangitis, lymphedema, wound healing and tissue repair, ischemia reperfusion injury, angina, myocardial infarctions such as acute myocardial infarctions, chronic heart conditions, heart failure such as congestive heart failure, and osteoporosis.

If, however, the molecule inhibits angiogenesis, an antagonist thereof would be used for treatment of those conditions where angiogenesis is desired.

Specific types of diseases are described herein, where the PRO-C-MG.2, PRO-C-MG.12, PRO-C-MG.45, PRO-C-MG.64 or PRO-C-MG.72 polypeptide or antagonists thereof may serve as useful for vascular-related drug targeting or as therapeutic targets for the treatment or prevention of the disorders. Atherosclerosis is a disease characterized by accumulation of plaques of intimal thickening in arteries, due to accumulation of lipids, proliferation of smooth muscle cells, and formation of fibrous tissue within the arterial wall. The disease can affect large, medium, and small arteries in any organ. Changes in endothelial and vascular smooth muscle cell function are known to play an important role in modulating the accumulation and regression of these plaques.

Hypertension is characterized by raised vascular pressure in the systemic arterial, pulmonary arterial, or portal venous systems. Elevated pressure may result from or result in impaired endothelial function and/or vascular disease.

Inflammatory vasculitides include giant cell arteritis, Takayasu's arteritis, polyarteritis nodosa (including the microangiopathic form), Kawasaki's disease, microscopic polyangiitis, Wegener's granulomatosis, and a variety of infectious-related vascular disorders (including Henoch-Schonlein prupura). Altered endothelial cell function has been shown to be important in these diseases.

Reynaud's disease and Reynaud's phenomenon are characterized by intermittent abnormal impairment of the circulation through the extremities on exposure to cold. Altered endothelial cell function has been shown to be important in this disease.

Aneurysms are saccular or fusiform dilatations of the arterial or venous tree that are associated with altered endothelial cell and/or vascular smooth muscle cells.

Arterial restenosis (restenosis of the arterial wall) may occur following angioplasty as a result of alteration in the function and proliferation of endothelial and vascular smooth muscle cells.

Thrombophlebitis and lymphangitis are inflammatory disorders of veins and lymphatics, respectively, that may result from, and/or in, altered endothelial cell function. Similarly, lymphedema is a condition involving impaired lymphatic vessels resulting from endothelial cell function.

Another use for the PRO-C-MG.2, PRO-C-MG.12, PRO-C-MG.45, PRO-C-MG.64 or PRO-C-MG.72 polypeptides herein or antagonists thereto is in the prevention or treatment of cancer, and preferably vascular tumors. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. The preferred cancers for treatment herein are breast, colon, lung, melanoma, ovarian, and others involving vascular tumors as noted above of tumor angiogenesis, which involves vascularization of a tumor to enable it to growth and/or metastasize. This process is dependent on the growth of new blood vessels. Further examples of preferred neoplasms and related conditions that involve tumor angiogenesis include breast carcinomas, gastric carcinomas, esophageal carcinomas, colorectal carcinomas, liver carcinomas, thecomas, arrhenoblastomas, cervical carcinomas, endometrial carcinoma, endometrial hyperplasia, endometriosis, fibrosarcomas, choriocarcinoma, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome. The family of benign vascular tumors, also included herein for treatment, are also characterized by abnormal proliferation and growth of cellular elements of the vascular system. For example, lymphangiomas are benign tumors of the lymphatic system that are congenital, often cystic, malformations of the lymphatics that usually occur in newborns. Cystic tumors tend to grow into the adjacent tissue. Cystic tumors usually occur in the cervical and axillary region. They can also occur in the soft tissue of the extremities. The main symptoms are dilated, sometimes reticular, structured lymphatics and lymphocysts surrounded by connective tissue. Lymphangiomas are assumed to be caused by improperly connected embryonic lymphatics or their deficiency. The result is impaired local lymph drainage (Griener et al., Lymphology 4:140-144 (1971)).

In one embodiment of the method of the invention, a PRO-C-MG.2, PRO-C-MG.12, PRO-C-MG.45, PRO-C-MG.64 or PRO-C-MG.72 antagonist is administered to a mammal, e.g. a human patient, in need thereof to reduce the tumor burden in the mammal. The compound, by inhibiting angiogenesis, is useful for the treatment of diseases or disorders characterized by undesirable excessive neovascularization, including by way of example tumors, and especially solid malignant tumors as mentioned herein, and non-neoplastic disorders including angina, myocardial infarctions such as acute myocardial infarctions, and heart failure such as congestive heart failure, psoriasis, diabetic and other proliferative retinopathies including retinopathy of prematurity, retrolental fibroplasia, neovascular glaucoma, thyroid hyperplasias (including Grave's disease), comeal and other tissue transplantation, chronic inflammation, lung inflammation, nephrotic syndrome, preeclampsia, ascites, pericardial effusion (such as that associated with pericarditis), pleural effusion, rheumatoid arthritis, atherosclerosis, hemangiomas, obesity, and age-related macular degeneration.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the PRO-C-MG.2, PRO-C-MG.12, PRO-C-MG.45, PRO-C-MG.64 or PRO-C-MG.72 polypeptides or antagonist thereto is expected to be useful in reducing the severity of AMD.

Healing of trauma such as wound healing and tissue repair is also a targeted use for the PRO-C-MG.2, PRO-C-MG.12, PRO-C-MG.45, PRO-C-MG.64 or PRO-C-MG.72 polypeptides herein or their antagonists. Formation and regression of new blood vessels is essential for tissue healing and repair. This category includes bone, cartilage, tendon, ligament, and/or nerve tissue growth or regeneration, as well as wound healing and tissue repair and replacement, and in the treatment of bums, incisions, and ulcers. A PRO-C-MG.2, PRO-C-MG.12, PRO-C-MG.45, PRO-C-MG.64 or PRO-C-MG.72 polypeptide or antagonist thereof that induces cartilage and/or bone growth in circumstances where bone is not normally formed has application in the healing of bone fractures and cartilage damage or defects in humans and other animals. Such a preparation employing a PRO-C-MG.2, PRO-C-MG.12, PRO-C-MG.45, PRO-C-MG.64 or PRO-C-MG.72 polypeptide or antagonist thereof may have prophylactic use in closed as well as open fracture reduction and also in the improved fixation of artificial joints. De novo bone formation induced by an osteogenic agent contributes to the repair of congenital, trauma-induced, or oncologic, resection-induced craniofacial defects, and also is useful in cosmetic plastic surgery. PRO-C-MG.2, PRO-C-MG.12, PRO-C-MG.45, PRO-C-MG.64 or PRO-C-MG.72 polypeptides or antagonists thereto may also be useful to promote better or faster closure of non-healing wounds, including without limitation pressure ulcers, ulcers associated with vascular insufficiency, surgical and traumatic wounds, and the like.

It is expected that a PRO-C-MG.2, PRO-C-MG.12, PRO-C-MG.45, PRO-C-MG.64 or PRO-C-MG.72 polypeptide or antagonist thereto may also exhibit activity for generation or regeneration of other tissues, such as organs (including, for example, pancreas, liver, intestine, kidney, skin, or endothelium), muscle (smooth, skeletal, or cardiac), and vascular (including vascular endothelium) tissue, or for promoting the growth of cells comprising such tissues. Part of the desired effects may be by inhibition or modulation of fibrotic scarring to allow normal tissue to regenerate.

A PRO-C-MG.2, PRO-C-MG.12, PRO-C-MG.45, PRO-C-MG.64 or PRO-C-MG.72 polypeptide herein or antagonist thereto may also be useful for gut protection or regeneration and treatment of lung or liver fibrosis, reperfusion injury in various tissues, and conditions resulting from systemic cytokine damage. Also, the PRO-C-MG.2, PRO-C-MG.12, PRO-C-MG.45, PRO-C-MG.64 or PRO-C-MG.72 polypeptide or antagonist thereto may be useful for promoting or inhibiting differentiation of tissues described above from precursor tissues or cells, or for inhibiting the growth of tissues described above.

A PRO-C-MG.2, PRO-C-MG.12, PRO-C-MG.45, PRO-C-MG.64 or PRO-C-MG.72 polypeptide or antagonist thereto may also be used in the treatment of periodontal diseases and in other tooth-repair processes. Such agents may provide an environment to attract bone-forming cells, stimulate growth of bone-forming cells, or induce differentiation of progenitors of bone-forming cells. A PRO-C-MG.2, PRO-C-MG.12, PRO-C-MG.45, PRO-C-MG.64 or PRO-C-MG.72 polypeptide herein or an antagonist thereto may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes, since blood vessels play an important role in the regulation of bone turnover and growth.

Another category of tissue regeneration activity that may be attributable to the PRO-C-MG.2, PRO-C-MG.12, PRO-C-MG.45, PRO-C-MG.64 or PRO-C-MG.72 polypeptide herein or antagonist thereto is tendon/ligament formation. A protein that induces tendon/ligament-like tissue or other tissue formation in circumstances where such tissue is not normally formed has application in the healing of tendon or ligament tears, deformities, and other tendon or ligament defects in humans and other animals. Such a preparation may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the PRO-C-MG.2, PRO-C-MG.12, PRO-C-MG.45, PRO-C-MG.64 or PRO-C-MG.72 polypeptide herein or antagonist thereto contributes to the repair of congenital, trauma-induced, or other tendon or ligament defects of other origin, and is also useful in cosmetic plastic surgery for attachment or repair of tendons or ligaments. The compositions herein may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions herein may also be useful in the treatment of tendinitis, carpal tunnel syndrome, and other tendon or ligament defects. The compositions may also include an appropriate matrix and/or sequestering agent as a carrier as is well known in the art.

Ischemia-reperfusion injury is another indication. Endothelial cell dysfunction may be important in both the initiation of, and in regulation of the sequelae of events that occur following ischemia-reperfusion injury.

Rheumatoid arthritis is a further indication. Blood vessel growth and targeting of inflammatory cells through the vasculature is an important component in the pathogenesis of rheumatoid and sero-negative forms of arthritis.

In view of the above, the PRO-C-MG.2, PRO-C-MG.12, PRO-C-MG.45, PRO-C-MG.64 or PRO-C-MG.72 polypeptides or agonists or antagonists thereof described herein, which are shown to alter or impact endothelial cell function, proliferation, and/or form, are likely to play an important role in the etiology and pathogenesis of many or all of the disorders noted above, and as such can serve as therapeutic targets to augment or inhibit these processes or for vascular-related drug targeting in these disorders.

M. Administration Protocols, Schedules, Doses, and Formulations

The molecules herein and agonists and antagonists thereto, including antigene compounds, are pharmaceutically useful as a prophylactic and therapeutic agent for various disorders and diseases as set forth above. Antigene compounds, such as antisense oligonucleotides, are more preferably formulated and administered as discussed above.

Therapeutic compositions of the PRO-C-MG.2, PRO-C-MG.12, PRO-C-MG.45, PRO-C-MG.64 or PRO-C-MG.72 polypeptides or agonists or antagonists are prepared for storage by mixing the desired molecule having the appropriate degree of purity with optional pharmaceutically acceptable carriers, excipients, or stabilizers (Remington's Pharmaceutical Sciences, 16th edition, Osol, A. ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; lipids such as cationic lipids; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of antagonist include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The PRO-C-MG.2, PRO-C-MG.12, PRO-C-MG.45, PRO-C-MG.64 or PRO-C-MG.72 polypeptides or agonists or antagonists will typically be formulated in such vehicles at a concentration of about 0.1 mg/ml to 100 mg/ml.

PRO-C-MG.2, PRO-C-MG.12, PRO-C-MG.45, PRO-C-MG.64 or PRO-C-MG.72 polypeptide or antagonist to be used for in vivo administration must be sterile. This is readily accomplished by filtration through sterile filtration membranes, prior to or following lyophilization and reconstitution. Therapeutic compositions herein generally are placed into a container having a sterile access port, for example, an intravenous solution bag or vial having a stopper pierceable by a hypodermic injection needle. PRO-C-MG.2, PRO-C-MG.12, PRO-C-MG.45, PRO-C-MG.64 or PRO-C-MG.72 polypeptide ordinarily will be stored in lyophilized form or in solution if administered systemically. If in lyophilized form, PRO-C-MG.2, PRO-C-MG.12, PRO-C-MG.45, PRO-C-MG.64 or PRO-C-MG.72 polypeptide or antagonist thereto is typically formulated in combination with other ingredients for reconstitution with an appropriate diluent at the time for use. An example of a liquid formulation of PRO-C-MG.2, PRO-C-MG.12, PRO-C-MG.45, PRO-C-MG.64 or PRO-C-MG.72 polypeptide or antagonist is a sterile, clear, colorless unpreserved solution filled in a single-dose vial for subcutaneous injection. Preserved pharmaceutical compositions suitable for repeated use may contain, for example, depending mainly on the indication and type of polypeptide: a) PRO-C-MG.2, PRO-C-MG.12, PRO-C-MG.45, PRO-C-MG.64 or PRO-C-MG.72 polypeptide or agonist or antagonist thereto; b)a buffer capable of maintaining the pH in a range of maximum stability of the polypeptide or other molecule in solution, preferably about 4-8; c) a detergent/surfactant primarily to stabilize the polypeptide or molecule against agitation-induced aggregation; d) an isotonifier; e) a preservative selected from the group of phenol, benzyl alcohol and a benzethonium halide, e.g., chloride; and f) water.

If the detergent employed is non-ionic, it may, for example, be polysorbates (e.g., POLYSORBATE™ (TWEEN™) 20, 80, etc.) or poloxamers (e.g., POLOXAMER™ 188). The use of non-ionic surfactants permits the formulation to be exposed to shear surface stresses without causing denaturation of the polypeptide. Further, such surfactant-containing formulations may be employed in aerosol devices such as those used in a pulmonary dosing, and needleless jet injector guns (see, e.g., EP 257,956).

An isotonifier may be present to ensure isotonicity of a liquid composition of the PRO-C-MG.2, PRO-C-MG.12, PRO-C-MG.45, PRO-C-MG.64 or PRO-C-MG.72 polypeptide or antagonist thereto, and includes polyhydric sugar alcohols, preferably trihydric or higher sugar alcohols, such as glycerin, erythritol, arabitol, xylitol, sorbitol, and mannitol. These sugar alcohols can be used alone or in combination. Alternatively, sodium chloride or other appropriate inorganic salts may be used to render the solutions isotonic.

The buffer may, for example, be an acetate, citrate, succinate, or phosphate buffer depending on the pH desired. The pH of one type of liquid formulation of this invention is buffered in the range of about 4 to 8, preferably about physiological pH.

The preservatives phenol, benzyl alcohol and benzethonium halides, e.g., chloride, are known antimicrobial agents that may be employed.

Examples of pharmacologically acceptable salts of molecules that form salts and are useful hereunder include alkali metal salts (e.g., sodium salt, potassium salt), alkaline earth metal salts (e.g., calcium salt, magnesium salt), ammonium salts, organic base salts (e.g., pyridine salt, triethylamine salt), inorganic acid salts (e.g., hydrochloride, sulfate, nitrate), and salts of organic acid (e.g., acetate, oxalate, p-toluenesulfonate).

The route of administration is in accord with known methods, e.g. injection or infusion by intravenous, intraperitoneal, intracerebral, intracerobrospinal, intraocular, intraarticular, intrasynovial, intrathecal, intraarterial or intralesional routes, oral, topical administration, or by sustained release systems. The compounds of the invention are also suitably administered by intratumoral, peritumoral, intralesional, or perilesional routes, to exert local as well as systemic therapeutic effects. The intraperitoneal route is expected to be particularly useful, for example, in the treatment of ovarian tumors. The formulations can also be administered as repeated intravenous (i.v.), subcutaneous (s.c.), or intramuscular (i.m.) injections, or as aerosol formulations suitable for intranasal or intrapulmonary delivery (for intrapulmonary delivery see, e.g. EP 257,956). If a peptide or small molecule is employed as an antagonist or agonist, it is preferably administered orally in the form of a liquid or solid.

PRO-C-MG.2, PRO-C-MG.12, PRO-C-MG.45, PRO-C-MG.64 or PRO-C-MG.72 polypeptide can also be administered in the form of sustained-released preparations. Suitable examples of sustained-release preparations include semipermeable matrices of solid hydrophobic polymers containing the protein, which matrices are in the form of shaped articles, e.g., films, or microcapsules. Microencapsulation of recombinant proteins for sustained release has been successfully performed with human growth hormone (rhGH), interferon- (rhIFN-), interleukin-2, and MN rgp120, Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid (EP 133,988). The sustained-release formulations developed using poly-lactic-coglycolic acid (PLGA) polymer are preferred due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release PRO-C-MG.2, PRO-C-MG.12, PRO-C-MG.45, PRO-C-MG.64 or PRO-C-MG.72 polypeptide compositions also include liposomally entrapped PRO-C-MG.2, PRO-C-MG.12, PRO-C-MG.45, PRO-C-MG.64 or PRO-C-MG.72 polypeptides. Liposomes containing the PRO-C-MG.2, PRO-C-MG.12, PRO-C-MG.45, PRO-C-MG.64 or PRO-C-MG.72 polypeptide are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal therapy.

The therapeutically effective dose of PRO-C-MG.2, PRO-C-MG.12, PRO-C-MG.45, PRO-C-MG.64 or PRO-C-MG.72 polypeptide, agonist, or antagonist thereto will, of course, vary depending on such factors as the pathological condition to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect. The progress of this therapy is easily monitored by appropriate clinical diagnostic methods. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a PRO-C-MG.2, PRO-C-MG.12, PRO-C-MG.45, PRO-C-MG.64 or PRO-C-MG.72 polypeptide, agonist, or antagonist thereof is employed, normal dosage amounts can vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, can necessitate delivery in a manner different from that to another organ or tissue.

Another formulation comprises incorporating a compound of the invention into formed articles. Such articles can be used in modulating endothelial cell growth, angiogenesis, and tumor invasion and metastasis. The therapeutic method includes administering the composition topically, systemically, or locally as an implant or device. When administered, the therapeutic composition for use is in a pyrogen-free, physiologically acceptable form. Further, the composition may desirably be encapsulated or injected in a viscous form for delivery to the targeted site. Topical administration may be suitable for wound healing, tissue repair, and skin and oral lesions and cancers. A kit, contains at least an article of manufacture comprising a PRO-C-MG.2, PRO-C-MG.12, PRO-C-MG.45, PRO-C-MG.64 or PRO-C-MG.72 polypeptide or an agonist or an antagonist thereof useful for the diagnosis or treatment of the disorders described above, and a label. The article of manufacture can be a container, including, for example, bottles, vials, syringes, test tubes, implants, and pumps. The containers may be formed from a variety of materials such as glass or plastic. The container holds a composition that is effective for diagnosing or treating the condition and may have a sterile access port (for example, the container may be an intravenous solution bag or a vial having a stopper pierceable by a hypodermic injection needle). The active agent in the composition is a compound of the invention. The label on, or associated with, the container indicates that the composition is used for diagnosing or treating the condition of choice. The kit can further comprise a second container comprising a pharmaceutically-acceptable buffer, such as phosphate-buffered saline, Ringer's solution, and dextrose solution. It can further include other materials desirable from a commercial and user standpoint, including other buffers, diluents, filters, needles, syringes, and package inserts with instructions for use. The kit can also comprise a second or third container with another active agent as described herein.

N. Combination Therapies

The effectiveness of the PRO-C-MG.2, PRO-C-MG.12, PRO-C-MG.45, PRO-C-MG.64 or PRO-C-MG.72 polypeptide or an agonist or antagonist thereof in preventing or treating the disorder in question may be improved by administering the active agent serially or in combination with another agent that is effective for those purposes, either in the same composition or as separate compositions. Positive regulators of angiogenesis include aFGF, bFGF, TGF-α, TGF-β, HGF, TNF-α, angiogenin, IL-8, etc. (see for example, Folkman et al., J. Biol. Chem., 267: 10931-10934 (1992) and Klagsbrun et al., Annu. Rev. Physiol., 53: 217-239 (1991)) and VEGF (Ferrara et al., Endocr. Rev., 18: 4-25 (1997)). Negative regulators include thrombospondin (Good et al., Proc. Natl. Acad. Sci. USA., 87: 6624-6628 (1990)), the 16-kilodalton N-terminal fragment of prolactin (Clapp et al., Endocrinology, 133: 1292-1299 (1993)), angiostatin (O'Reilly et al. Cell, 79: 315-328 (1994)), and endostatin (O'Reilly et al., Cell, 88: 277-285 (1996)). The effectiveness of the agonist in preventing or treating disease may be improved by administering the agonist serially or in combination with yet another agent that is effective for those purposes, such as immunoadhesins, ribozymes, antisense agents, tumor necrosis factor (TNF), an antibody capable of inhibiting or neutralizing the angiogenic activity of acidic or basic fibroblast growth factor (FGF), vascular endothelial growth factor (VEGF), or hepatocyte growth factor (HGF), an antibody capable of inhibiting or neutralizing the coagulant activities of tissue factor, protein C, or protein S (see Esmon, et al., PCT Patent Publication No. WO 91/01753, published 21 Feb. 1991), an antibody capable of binding to HER2 receptor (see Hudziak, et al., PCT Patent Publication No. WO 89/06692, published 27 Jul. 1989), or one or more conventional therapeutic agents such as, for example, alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, corticosteroids and proteins such as angiostatin, endostatin, thrombospondin, and platelet factor 4. For example, vascularization of tumors can be blocked with combination therapy, in which one or more antagonists are administered to tumor-bearing patients at therapeutically effective doses as determined for example by observing necrosis of the tumor or its metastatic foci, if any. This therapy is continued until such time as no further beneficial effect is observed or clinical examination shows no trace of the tumor or any metastatic foci. The antagonist is administered, alone or in combination, with an auxiliary agent such as α-, β-, or γ-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, such as anti-protein C antibody, anti-protein S antibody, or C4b binding protein (Esmon, et al., PCT Patent Publication No. WO 91/01753). Such other agents may be present in the composition being administered or may be administered separately. Also, the antagonist is suitably administered serially or in combination with heat or radiological treatments, whether involving irradiation or administration of radioactive substances. Examples of chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228).

Since the auxiliary agents will vary in their effectiveness it is desirable to compare their impact on the tumor by matrix screening in conventional fashion. For example, the administration of an antagonist of the invention and auxiliary agent is repeated until the desired clinical effect is achieved. In instances where solid tumors are found in the limbs or in other locations susceptible to isolation from the general circulation, the therapeutic agents described herein are administered to the isolated tumor or organ. In a preferred embodiment, a FGF, platelet-derived growth factor (PDGF), or VEGF antagonist, such as an anti-FGF, anti-VEGF, or an anti-PDGF neutralizing antibody, is administered to the patient in conjunction with an antagonist compound of the invention. Treatment with an antagonist can be suspended during periods of wound healing or desirable vascularization, or alternatively an agonist of the invention can be used to promote such benefit.

For other indications, PRO-C-MG.2, PRO-C-MG.12, PRO-C-MG.45, PRO-C-MG.64 or PRO-C-MG.72 polypeptides or their agonists can be combined with other agents beneficial to the treatment of the defect, wound, or tissue in question. These agents include various growth factors such as EGF, PDGF, TGF-α or TGF-β, IGF, FGF, and CTGF, as discussed herein.

In addition, PRO-C-MG.2, PRO-C-MG.12, PRO-C-MG.45, PRO-C-MG.64 or PRO-C-MG.72 polypeptides or their antagonists used to treat cancer may be combined with cytotoxic, chemotherapeutic, or growth-inhibitory agents as identified above. Also, for cancer treatment, the PRO-C-MG.2, PRO-C-MG.12, PRO-C-MG.45, PRO-C-MG.64 or PRO-C-MG.72 polypeptide or antagonist thereof is suitably administered serially or in combination with radiological treatments, whether involving irradiation or administration of radioactive substances.

Certain preferred embodiments of the invention provide pharmaceutical compositions containing (a) one or more antigene compounds and (b) one or more other agents which function by a non-antigene mechanism, such as chemothreapeutic, angiogenic, or angiostatic agents as discussed. Two or more combined compounds may be used together or sequentially.

In another related embodiment, compositions of the invention may contain one or more antigene compounds, particularly oligonucleotides, targeted to a PRO-C-MG.2, PRO-C-MG.12, PRO-C-MG.45, PRO-C-MG.64 or PRO-C-MG.72 nucleic acid and one or more additional antigene compounds targeted to a second nucleic acid target. Numerous examples of antigene compounds are known in the art. Two or more combined compounds may be used together or sequentially. For example, Im et al., Cancer Research 59(4):895-900 (1999) reported inhibition of tumor growth using antsense VEGF. The recombinant adenoviral vector Ad5CMV carried the coding sequence of wild-type VEGF165 cDNA in an antisense orientation. Infection of U-87 MG malignant glioma cells with the vector resulted in reduction of the level of the endogenous VEGF mRNA and drastically decreased the production of the targeted secretory form of the VEGF protein. Treatment of s.c. human glioma tumors established in nude mice with intralesional injection of antisense VEGF vector inhibited tumor growth. Sharmia et al. (J. Clin. Invest. 102(8):1599-608 (1998)) reparted that by blocking perlecan expression by using either constitutive CMV-driven or doxycycline-inducible antisense constructs, growth of colon carcinoma cells was markedly attenuated. In both tumor xenografts induced by human colon carcinoma cells and tumor allografts induced by highly invasive mouse melanoma cells, perlecan suppression caused substantial inhibition of tumor growth and neovascularization. In is noted that the mouse system, in combination with an appropriate human tumor or tumor cell line xenograft or mouse allograft, also provides a rapid means to screen for maxmimally effective PRO-C-MG.2, PRO-C-MG.12, PRO-C-MG.45, PRO-C-MG.64 or PRO-C-MG.72 antigene compounds in vivo.

The effective amounts of the therapeutic agents administered in combination with the compounds of the invention will be at the physician's or veterinarian's discretion. Dosage administration and adjustment is done to achieve maximal management of the conditions to be treated. The dose will additionally depend on such factors as the type of the therapeutic agent to be used and the specific patient being treated. Typically, the amount employed, at least as a starting point, will be the same dose as that used, if the given therapeutic agent is administered without a compound of the invention.

According to the present invention, angiogenesis, vascular and neovascular conditions are particularly well-suited to antigene therapy (see e.g., Thomas et al. Radiographics 18(6):1373-94 (1998)). Accordingly, local gene transfer into the vascular wall offers a promising alternative to treat angiogenic-related diseases and disorders described herein. Blood vessels, and the vascular wall, are among the easiest targets for gene therapy because of ready accessibility and of novel percutaneous, catheter-based treatment methods. Vascular and interventional radiology techniques also are ideally suited for minimally invasive, readily monitored gene delivery, of either viral or nonviral vectors or of syntehetic oligo compounds. Recombinant genes can be delivered ex vivo and in vivo; the latter approaches can involve well-known open surgical, percutaneous injection, or endovascular catheter-based methods. Perforated, hydrogel-coated, and double balloon catheters can also be readily used. Catheter systems for gene transfer enable delivery of the vector to the precise anatomic location with transfection limited to the cells of interest and will minimize shedding of the vector to distal sites, systemic effects of the therapeutic agent, and morbidity from the delivery method. On the other hand, gene transfer to the artery wall can also be accomplished from adventitia, and in some situations intramuscular gene delivery. Promising therapeutic effects have been obtained in animal models of restenosis with the transfer of genes for vascular endothelial growth factor, fibroblast growth factor, thymidine kinase, p53, bcl-x, nitric oxide synthase and retinoblastoma. Also, growth arrest homeobox gene and antisense oligonucleotides against transcription factors or cell cycle regulatory proteins have produced beneficial therapeutic effects. Antiangiogenic tumor therapies using antigen technology can provide broad-spectrum action, low toxicity, and, in the case of direct endothelial targeting, an absence of drug resistance. Gene therapy offers a potential way to achieve sustained therapeutic release of potent antiangiogenic substances. An alternative for longer term administration (or as combination therapy) are recombinant vectors carrying antisense genes. For example, adeno-associated virus (rAAV) vectors carrying genes coding for angiostatin, endostatin, and an antisense mRNA species against vascular endothelial growth factor (VEGF), efficiently transduced three human tumor cell lines tested. Transduction with an rAAV-encoding antisense VEGF mRNA inhibited the production of endogenous tumor cell VEGF (Nguyen et al., Cancer Research, 58(24):5673-7 (1998)). The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

Isolation of cDNA Clones Encoding a Human PRO-C-MG.2, PRO-C-MG.12, PRO-C-MG.45, PRO-C-MG.64 or PRO-C-MG.72

Formation of Three-Dimensional Collagen Gels: Tube formation by endothelial cells is a critical process in the development of a blood vessel during angiogenesis and vasculogenesis. Human umbilical cord endothelial cells (HUVEC) undergoing tube formation in collagen gels in the presence of growth factors, mimic the angiogenic environment of endothelial cells in vivo, providing a well-accepted system for angiogenisis and vasculogenesis, both in normal and neoplastic conditions. The three dimensional gel is prerequisite for the differentiation and fusion of endothelial cells into tubes, as HUVECs grown on the surface of gelatin or on plastic do not undergo tube-formation.

In brief, HUVECs were grown under various conditions, inductive or non-inductive to tube formation, either on collagen film (non-inductive) or in collagen gels (inductive), with or without the addition of growth factors to simulate induction by normal angiogenic factors or by tumor-derived factors. Differential cDNA screening was used to identify genes critical to this process. The particular method used to quantitate endothelial cell gene expression was Quantitative Expression Analysis (QEA; U.S. Pat. No. 5,871,697). HUVEC total RNA was prepared, followed by mRNA purification and double stranded cDNA synthesis. The cDNA was digested with restriction enzyme pairs to produce cDNA fragments, which were then ligated with linkers. Primer pairs bearing the specific sequences of the linkers were used to amplify the restricted products in a PCR reaction. Quantification and identification of amplified products revealed modulated genes, thus identifying genes critical to angiogenesis.

Tube formation was achieved as follows. Collagen gels were formed by mixing together an ice-cold gelation solution of a 100:27.7:50:10:750:62.5 ratio (by volume) of a ten-fold-concetrated M199 stock. water, 0.53 M NaHCO3, 200 mM L-glutamine, type I collagen, and 0.1 M NaOH. This was mixed with HUVEC cells (in 1× basal medium at a concentration of 3×10⁶ cells/ml) at a ratio of 4 volumes gelation solution to 1 volume of cells. The gels were allowed to form by incubation in a CO₂-free incubator at 37° C. for 30 min to 1 hour. The gels were then overlaid with 1× basal medium consisting of M199 supplemented with 1% FBS, 1×ITS, 2 mM L-glutamine, 50 mg/ml ascorbic acid, 26.5 mM NaHCO3, 100 U/ml penicillin and 100 U/ml streptomycin. In the tube-forming experiments, the culture media was supplemented with 80 nM PMA, 40 ng/ml bFGF and 40 ng/ml VEGF. In a parallel set of experiments, endothelial cells were cultured on the surface of type I collagen, or on pig skin gelatin (DIFCO, USA) in 1× basal medium consisting of M199 supplemented with 1% FBS, 1×ITS, 2 mM L-glutamine, 50 mg/ml ascorbic acid, 26.5 mM NaHCO3, 100 U/ml penicillin and 100 U/ml streptomycin, without or with 80 nM PMA, 40 ng/ml bFGF and 40 ng/ml VEGF. For the differential cDNA screening experiment (by QEA, also referred to as GeneCalling™, Curagen Corp., USA), mRNA was isolated from cells incubated in the above conditions for 4 hr, 24 hr and 48 hr.

mRNA was isolated and cDNA synthesized as follows. Media was aspirated from the surface of the collagen gels and the gels were scraped into a 50 ml polypropylene tube containing 3 volumes of Tri-Reagent-LS (Molecular Research Center, Cincinnati, Ohio). The tubes were incubated 10 min at 23° C. with intermittent gentle agitation. The tubes were stored at −80° C. until all experimental samples had been collected. The tubes were then thawed at room temperature and the mRNA extracted following manufacturer's specifications. The RNA pellets were resuspended in DEPC-treated water and RNA content quantified spectroscopically at 260 nm. RNA samples were stored −20° C. Samples used for GeneCalling™ analysis were shipped on dry ice to Curagen Corp. (New Haven, Conn.). Samples from time points of 4, 24 and 48 hrs were used for the GeneCalling™ analysis, and in a separate experiments, samples cells grown in collagen gels and on the surface of type I collage in 1× basal medium supplemented with 80 nM PMA, 40 ng/ml bFGF and 40 ng/ml VEGF from time points of 30 min, and 2, 4, 8, 16, 24, 38 and 46.5 hrs were prepared for Taqman PCR confirmation. For the quantitative expression analysis, contaminating DNA was removed by treatment of the isolated RNA with DNAse I (Promega, Madison, Wis.). Poly-A+RNA was prepared by fractionation of total RNA using an mRNA purification kit that utilized biotinylated oligo-dT-Streptavidin magnetic bead method (MPG, LincolnPark, N.J.) followed by cDNA synthesis by reverse transcription of oligo-dT primed mRNA (Superscript II, Life Technologies) and second strand synthesis. Terminal phosphate removal was achieved by treatment with Artic Shrimp Alkaline Phosphtase (Amersham Life Sciences, Piscataway, N.J.) followed by purification of cDNA by phenol-chlorofonm extraction. Yield of cDNA was quantitated by fluorometry using PicoGreen dye (Molecular Probes, Eugene Oreg.). Double stranded DNA was digested using pairs of restriction enzymes with 6 base-pair recognition sites. More than 48 enzyme pairs were used and were chosen such that a representative coverage of most of the possible sequences in a given DNA sample was achieved. PCR amplification using specific linkers was carried out as described in U.S. Pat. No. 5,871,697. The final DNA products were denatured by heating to 96° C. and electrophoresed on ultra-thin polyacrylamide gels under denaturing conditions in 6M urea. PCR products were visualized by the presence of FAM label on the product using a multi-color laser excitation Niagara (Curagen Corp., New Haven Conn.) imaging system.

GeneCalling analysis used a fully integrated Web-based interactive bioinformatics data gathering and analysis suite called “GeneScape.” The data obtained from Niagara gels were GeneCalled against public and proprietary databases using present statistical and mathematical criteria (U.S. Pat. No. 5,871,697) and a gene list was generated from the cDNA fragment data that is a list of likely genes that the cDNA fragment can belong to based on the size of the fragment and the position of the restriction enzyme pair that produced it in the known sequence. If a gene candidate could not be obtained, the cDNA fragment was designated as belonging to a putative novel gene.

A GeneCall was defined as the probability of a cDNA fragment belonging to a known gene. GeneCalls were confirmed in a poisoning reaction where the known sequence of the likely gene of interest is used to design poisoning primers as previously described (U.S. Pat. No. 5,871,697; Shimkets et al. Nature Biotechnology 17(8):798-803 (1999)). Ablation of the cDNA fragment of interest confirmed that the cDNA fragment belonged to the gene for which the primer was designed.

If no GeneCall was obtained for a cDNA fragment, the putative novel cDNA fragment was eluted from, and subcloned into E. coli using standard TA-cloning vector (Invitrogen, Palo Alto, Calif.). The putative novel cDNA fragment was then sequenced and the resulting sequence used to design poison primers for confirmation as described above.

To confirm the expression data from GeneCalling by an independent technique, Quantitative RT-PCR (Taqman), in which gene specific PCR oligonucleotide primer pairs and oligonucleotide probes labeled with a reporter fluorescent dye at the 5′ end and quencher fluorescent dye at the 3′ end were designed using the Oligo 4.0 software (National Bioscience, Plymouth Minn.). Total RNA (50 ng) was added to a 50 ml RT-PCR reaction mixture according to the manufacturer's protocol (Roche Molecular Systems Inc., Branchburg, N.J.). The thermal cycling conditions included 1 cycle at 48° C. for 30 min, 1 cycle at 95° C. for 10 min, 40 cycles at 95° C. for 15s, annealing at 60° C. for 1 min, and a final hold at 25° for 2 min. Standard curves for the expression of each gene were generated by serial dilution of a standard preparation of total RNA isolated from quiescent HUVEC grown in monolayer culture. Data were expressed as the fold induction normalized to the same gene from quiescent HUVEC RNA.

The GeneCalling process was used in the selection (gating) of differentially expressed genes in tube formation. The experimental design was based on the observation that endothelial cells grown on the surface of type I collagen in 1× basal medium supplemented with 80 nM PMA, 40 ng/ml bFGF and 40 ng/ml VEGF do not form tubes, but rather remain as a monolayer. This result also occurs if the cells are grown on gelatin, a form of denatured collagen. However, if the cells are suspended in a three dimensional collagen gel, and grown in 1× basal media supplemented with 80 nM PMA, 40 ng/ml bFGF and 40 ng/ml VEGF, the cells undergo a synchronous differentiation into an interconnected tube like network. The tubular structures contain lumen-like structures. At 4 hours, large intracellular vacuoles are forming, but the cells are still round. At 24 hrs, the cells have become elongated and many cells are touching other cells. By 48 hrs, the cells have become interconnected and share common lumens. To select for genes that play a role in this differentiation, an array of GeneCalling differences was set up such that cDNA fragments that changed more than 2 fold between 24 hours and 4 hours between 48 hours and 4 hours, or between 48 hours and 24 hrs, in the 3-D gel environment, but which were unchanged or changed less than 2 fold in the 2D (surface of type I collagen or gelatin) environment at the same pair-wise time comparisons were preferentially selected and identified. In addition, those cDNA fragments which demonstrate large (greater than 8 fold) changes in gene expression vere also identified.

Full length cDNAs corresponding to the differentially expressed genes identified by their GeneCalled fragments were prepared and sequenced as follows. An oligo dT primed cDNA library was prepared from mRNA isolated from human HUVEC cells as above, using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to greater than 1000 bp and the SalI/NotI linkered cDNA was cloned into XhoI/NotI cleaved vector. pRK5D is a cloning vector that has an sp6 transcription initiation site followed by an SfiI restriction enzyme site preceding the XhoI/NotI cDNA cloning sites.

Oligonucleotides probes based upon the above described GeneCall fragment sequence were then synthesized to identify by PCR a cDNA library that contained the sequence of interest, and to use as probes to isolate a clone of the full-length coding sequence for the differentially expressed gene of interest. Forward and reverse PCR primers generally range from 20 to 30 nucleotides and are often designed to give a PCR product of about 100-1000 bp in length. The probe sequences are typically 40-55 bp in length. In order to screen several libraries for a full-length clone, DNA from the libraries was screened by PCR amplification, as per Ausubel et al., Current Protocols in Molecular Biology,John Wiley and Sons (1997), with the PCR primer pair. A positive library was then used to isolate clones encoding the gene of interest using the probe oligonucleotide and one of the primer pairs.

PRO-C-MG.2.

The initial GeneCall assembled fragment sequence was SEQ ID No. 5. To obtain the full-length clone, oligonucleotide probes based on this sequence were as follows:

(SEQ ID NO:6)

forward PCR primer
5′ GGAGGACACGGTGCCGCTGACAGC 3′

(SEQ ID NO:8)

reverse PCR primer
5′ GTTTTCCAGAGAAATTCCTCTTTGCACTCGA 3′

(SEQ ID NO:7)

hybridization probe
5′ GCGATCGAGGCGAGCCAGAGCCTGCAGTCCCACACGGAATATATTA
TTCGA 3′

A full length clone was identified that contained a single open reading frame with an apparent translational initiation site at nucleotide positions 66-68 and a stop signal at nucleotide positions 1794-1796 (SEQ ID NO:1). The predicted polypeptide is 577 amino acids long, has a calculated molecular weight of approximately 64935.06 daltons and an estimated pI of approximately 9.94. Analysis of the full-length PRO-C-MG.2 sequence shown in SEQ ID NO:2 evidences the presence of a variety of important polypeptide domains. The locations given for those important polypeptide domains are approximate as described: cAMP- and cGMP-dependent protein kinase phosphorylation site at amino acid positions 54-58, 441-445, and 464-468; casein kinase II phosphorylation site at amino acid positions 32-36, 57-61, 110-114, 179-183, 190-194, 216-220, 233-237, 402-406, 452-456, 470-474; tyrosine kinase phosphorylation site at amino acid positions 116-125 and 117-125; N-myristoylation site at amino acid positions 489-495, 545-551, and 549-555; leucine zipper pattern at amino acid positions 289-311; a PX kinase domain at amino acid position 16-122; and, a pkinase domain from amino acid positions 230-284. This gene had a greater than 4-fold increase in gene expression as determend by the GeneCalling approach. clone 12 is +2.5× by GeneCalling

Clone DNA-C-MG.2-1776 has been deposited with ATCC on Sep. 28, 1999, and is assigned ATCC deposit no. PTA-799.

PRO-C-MG.12.

The initial GeneCall assembled fragment sequence was SEQ ID No. 9. To obtain the full-length clone, oligonucleotide probes based on this sequence were as follows:

(SEQ ID NO:10)

forward PCR primer
5′ GACCTATTGGGACACCTTCTGGAG 3′

(SEQ ID NO:12)

reverse PCR primer
5′ CTTGGTCAGACGAGAGGAGCTGATC 3′

(SEQ ID NO:11)

hybridization probe
5′ CCCGCCTAGTGCCAAGCAACCCTCCAAGATGCTAGTTATCAAA 3′

A full length clone was identified that contained a single open reading frame with an apparent translational initiation site at nucleotide positions 465-467 and a stop signal at nucleotide positions 1884-1886 (SEQ ID NO:3). The predicted polypeptide is 474 amino acids long, has a calculated molecular weight of approximately 52573.30 daltons and an estimated pI of approximately 6.66. Analysis of the full-length PRO-C-MG.12 sequence shown in SEQ ID NO:4 evidences the presence of a variety of important polypeptide domains. The locations given for those important polypeptide domains are approximate as described: cAMP- and cGMP-dependent protein kinase phosphorylation site at amino acid positions 199-203 and 316-320; casein kinase II phosphorylation site at amino acid positions 61-65, 81-85, 202-206, 266-270, 292-296, 328-332, 353-357, 411-415, 458-462, 463-467, 467-471, and 468-472; N-myristoylation site at amino acid positions 112-118, 122-128, 177-183, 218-224, 224-230, 262-268, 287-293, and 364-370; and a potential autocatalytic peptide splicing site at position 99-104 (LPRGhD; see Gu et al., J. Biol. Chem. 268(10):7372-81 (1993)). This gene had a greater than 2.5-fold increase in gene expression as determined by the GeneCalling approach.

Clone DNA-C-MG.12-1776 has been deposited with ATCC on Sep. 28, 1999, and is assigned ATCC deposit no. PTA-798.

An analysis of the Dayhoff database (version 35.45 SwissProt 35), using the ALIGN-2 sequence alignment analysis of the full-length sequence shown in SEQ ID NO:4, evidenced sequence identity between the PRO-C-MG.12 amino acid sequence and the following Dayhoff sequences: Accession P_Y00281. P_Y00281 was reported as a 337amino acid long human allegedly secreted protein encoded by gene 24 in WO9906423. This 337 amino acid sequence matches PRO-C-MG.12 from amino acid position 138 to 474. WO9906423 alleges that P_Y00281 gene maps to chormosome 1, and was expressed primarily in brain, and to a lesser extent in ovaries and activated T-cells, and consequently was alleged to find use primarily in brain neurodegenerative disorders, immune deficiencies and reproduction.

PRO-C-MG.45.

The initial GeneCall assembled fragment sequence was SEQ ID No. 24. This was similar to EST accession # AA461480. To obtain a longer sequence, SEQ ID NO: 24 was used to search public EST and other sequence databases (e.g., GenBank). The search was performed using the computer program BLAST or BLAST-2 (Altschul et al., Methods in Enzymology, 266:460-480 (1996)). Comparisons with a BLAST score of preferably 70 (or in some cases, 90) or greater that did not encode known proteins were clustered and assembled into a consensus DNA sequences with the program “phrap” (Phil Green, University of Washington, Seattle, Wash.). Using this homology screen, consensus DNA sequences were assembled relative to other identified EST sequences using phrap. In addition, the consensus DNA sequences obtained were often (but not always) extended using repeated cycles of BLAST and phrap to extend the consensus sequence as far as possible using the sources of EST sequences discussed above.

From either the GeneCalled sequence or the consensus sequence or both, oligonucleotides are then synthesized and used to identify by PCR a cDNA library that contains the sequence of interest and for use as probes to isolate a clone of the full-length coding sequence for a PRO polypeptide. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs. To obtain the full-length clone, preferred oligonucleotide primers are preferably as follows:
forward PCR primer 5′ atgcagttct ttttcaactt cc (SEQ ID NO:26)
reverse PCR primer 5′ ctagagacca atctaagtaa 3′ (SEQ ID NO:27)
   The probe can be a unique region preferably from about 19 to about 100 base pairs.

The cDNA libraries used to isolate the cDNA clones are constructed by standard methods using commercially available reagents, such as those from Invitrogen, San Diego, Calif. The cDNA is primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

PRO-C-MG.64.

The initial GeneCall assembled fragment sequence was SEQ ID No. 5. This was similar to EST accession # AA913939. To obtain a longer sequence, SEQ ID NO: 24 was used to search public EST and other sequence databases (e.g., GenBank). The search was performed using the computer program BLAST or BLAST-2 (Altschul et al., Methods in Enzymology, 266:460-480 (1996)). Comparisons with a BLAST score of preferably 70 (or in some cases, 90) or greater that did not encode known proteins were clustered and assembled into a consensus DNA sequences with the program “phrap” (Phil Green, University of Washington, Seattle, Wash.). Using this homology screen, consensus DNA sequences were assembled relative to other identified EST sequences using phrap. In addition, the consensus DNA sequences obtained were often (but not always) extended using repeated cycles of BLAST and phrap to extend the consensus sequence as far as possible using the sources of EST sequences discussed above. To obtain the full-length clone, preferred oligonucleotide primers are as follows:

| | |
|---|---|
| forward PCR primer | |
| 5′ atggtggagtggaggacctg | (SEQ ID NO:28) |
| reverse PCR primer | |
| 5′ ctccaacacc aagtactctt ga 3′ | (SEQ ID NO:29) |

The probe can be a unique region preferably from about 19 to about 100 base pairs.

The cDNA libraries use to isolate the cDNA clones are constructed by standard methods using, commercially available reagents, such as those from Invitrogen, San Diego, Calif. The cDNA is primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

PRO-C-MG.72.

The initial GeneCall assembled fragment sequence was SEQ ID No. 5. This was similar to EST accession # aa771960. To obtain the full-length clone, oligonucleotide probes based on this sequence were as follows:

(SEQ ID NO:20)

forward PCR primer
5′ gctgcttcttggttggaagattctgg 3′

(SEQ ID NO:21)

reverse PCR primer
5′ ccagaatcttccaaccaagaagcagc 3′

hybridization probe 5′ gcatcatgctgtttgacactttcccaattaaaagtcccttcataaaactt tgc 3′ (SEQ ID NO:22), and a reverse hybridization probe was ggagctgccattagaatcaagaatctttgc (SEQ ID NO:23)

A full length clone was identified that contained a single open reading frame with an apparent translational initiation site at nucleotide positions 71-73 and a stop signal at nucleotide positions 2060-2062 (SEQ ID NO:13). The predicted polypeptide is 663 amino acids long. Analysis of the full-length PRO-C-MG.72 sequence shown in SEQ ID NO:14 evidences the presence of a variety of important polypeptide domains. The most interesting of which is the RhoGap domain approximate from about amino acid 343 to about 494, as determined by the Pfam algorithm, giving a very significant E-value of 8.2×10⁻²⁸ and a score of 105.8. Accordingly, PRO-C-MG.72 is believed to have activity as a GTPase-activating protein, preferably of the Rho-type. In this regard a particularly important regions, that contain GTPase-activating active domains, are from about amino acid position 206 to about 553, to about 307 to about 500, and to about 341 to about 533, in SEQ ID NO:14.

### Example 2

Use of PRO-C-MG.2, PRO-C-MG.12, PRO-C-MG.45, PRO-C-MG.64 or PRO-C-MG.72 as a Hybridization Probe

The following method describes use of a nucleotide sequence encoding PRO-C-MG.2, PRO-C-MG.12, PRO-C-MG.45, PRO-C-MG.64 or PRO-C-MG.72 as a hybridization probe.

DNA comprising the coding sequence of full-length or mature PRO-C-MG.2, PRO-C-MG.12, PRO-C-MG.45, PRO-C-MG.64 or PRO-C-MG.72 is employed as a probe to screen for homologous DNAs (such as those encoding naturally-occurring variants of PRO-C-MG.2, PRO-C-MG.12, PRO-C-MG.45, PRO-C-MG.64 or PRO-C-MG.72) in human tissue cDNA libraries or human tissue genonic libraries.

Hybridization and washing of filters containing either library DNAs is performed under the following high stringency conditions. Hybridization of radiolabeled PRO-C-MG.2, PRO-C-MG.12, PRO-C-MG.45, PRO-C-MG.64 or PRO-C-MG.72-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performied in an aqueous solution of 0.1×SSC and 0.1% SDS at 42° C.

DNAs having a desired sequence identity with the DNA encoding full-length native sequence PRO-C-MG.2, PRO-C-MG.12, PRO-C-MG.45, PRO-C-MG.64 or PRO-C-MG.72 can then be identified Using standard techniques known in the art.

### Example 3

Expression of PRO-C-MG.2, PRO-C-MG.12, PRO-C-MG.45, PRO-C-MG.64 or PRO-C-MG.72 in E. coli

This example illustrates preparation of an unglycosylated form of PRO-C-MG.2, PRO-C-MG.12, PRO-C-MG.45, PRO-C-MG.64 or PRO-C-MG.72 by recombinant expression in E. coli.

The DNA sequence encoding PRO-C-MG.2, PRO-C-MG.12, PRO-C-MG.45, PRO-C-MG.64 or PRO-C-MG.72 is initially amplified using selected PCR primers. The primers should contain restriction enzyme sites which correspond to the restriction enzyme sites on the selected expression vector. A variety of expression vectors can be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO-C-MG.2, PRO-C-MG.12, PRO-C-MG.45, PRO-C-MG.64 or PRO-C-MG.72 coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture can subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO-C-MG.2, PRO-C-MG.12, PRO-C-MG.45, PRO-C-MG.64 or PRO-C-MG.72 protein can then be purified using a metal chelating column under conditions that allow tight binding of the protein.

PRO-C-MG.2, PRO-C-MG.12, PRO-C-MG.45, PRO-C-MG.64 or PRO-C-MG.72 can be expressed in E. coli in a poly-His tagged form, using the following procedure. The DNA encoding PRO-C-MG.2, PRO-C-MG.12, PRO-C-MG.45, PRO-C-MG.64 or PRO-C-MG.72 is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) Ion galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂ SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1 M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO-C-MG.2, PRO-C-MG.12, PRO-C-MG.45, PRO-C-MG.64 or PRO-C-MG.72 polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 4

Expression of PRO-C-MG.2, PRO-C-MG.12, PRO-C-MG.45, PRO-C-MG.64 or PRO-C-MG.72 in Mammalian Cells

This example illustrates preparation of a potentially glycosylated form of PRO-C-MG.2, PRO-C-MG.12, PRO-C-MG.45, PRO-C-MG.64 or PRO-C-MG.72 by recombinant expression in mammalian cells.

The vector, pRK5 (see EP 307,247, published Mar. 15, 1989), is employed as the expression vector. Optionally, the PRO-C-MG.2, PRO-C-MG.12, PRO-C-MG.45, PRO-C-MG.64 or PRO-C-MG.72 DNA is ligated into pRK5 with selected restriction enzymes to allow insertion of the PRO-C-MG.2, PRO-C-MG.12, PRO-C-MG.45, PRO-C-MG.64 or PRO-C-MG.72 DNA using ligation methods such as described in Sambrook et al., supra. The resulting vector is called pRK5-PRO-C-MG.2, PRO-C-MG.12, PRO-C-MG.45, PRO-C-MG.64 or PRO-C-MG.72.

In one embodiment, the selected host cells can be HUVEC cells as described above, using the vectors and transfection methods described herein for other mammalian cells. Transfected HUVEC cells over-expressing PRO-C-MG.2, PRO-C-MG.12, PRO-C-MG.45, PRO-C-MG.64 or PRO-C-MG.72 or expressing PRO-C-MG.2, PRO-C-MG.12, PRO-C-MG.45, PRO-C-MG.64 or PRO-C-MG.72 antisense are tested, for example, in the tube formation assay.

In one embodiment, the selected host cells can be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO-C-MG.2, PRO-C-MG.12, PROC-MG.45, PRO-C-MG.64 or PRO-C-MG.72 DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel can be dried and exposed to film for a selected period of time to reveal the presence of PRO-C-MG.2, PRO-C-MG.12, PRO-C-MG.45, PRO-C-MG.64 or PRO-C-MG.72 polypeptide. The cultures containing transfected cells can undergo further incubation (in serum free medium) and the medium is tested in selected bioassays.

In an alternative technique, PRO-C-MG.2, PRO-C-MG.12, PRO-C-MG.45, PRO-C-MG.64 or PRO-C-MG.72 can be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO-C-MG.2, PRO-C-MG.12, PRO-C-MG.45, PRO-C-MG.64 or PRO-C-MG.72 DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO-C-MG.2, PRO-C-MG.12, PRO-C-MG.45, PRO-C-MG.64 or PRO-C-MG.72 can then be concentrated and purified by any selected method, such as dialysis and/or column chromatography.

In another embodiment, PRO-C-MG.2, PRO-C-MG.12, PRO-C-MG.45, PRO-C-MG.64 or PRO-C-MG.72 can be expressed in CHO cells. The pRK5-PRO-C-MG.2, PRO-C-MG.12, PRO-C-MG.45, PRO-C-MG.64 or PRO-C-MG.72 can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO-C-MG.2, PRO-C-MG.12, PRO-C-MG.45, PRO-C-MG.64 or PRO-C-MG.72 polypeptide, the culture medium can be replaced with serum free medium. Preferably, the cultures are incubated for about 6 days, and then the conditioned medium is harvested. The medium containing the expressed PRO-C-MG.2, PRO-C-MG.12, PRO-C-MG.45, PRO-C-MG.64 or PRO-C-MG.72 can then be concentrated and purified by any selected method. Epitope-tagged PROC-MG.2, PROC-MG.12, PRO-C-MG.45, PRO-C-MG.64 or PROC-MG.72 can also be expressed in host CHO cells. The PROC-MG.2, PRO-C-MG.12, PRO-C-MG.45, PRO-C-MG.64 or PRO-C-MG.72 can be subcloned out of the pRK5 vector. The subclone insert can undergo PCR to fuse in frame with a selected epitope tag such as a poly-his tag into a Baculovirus expression vector. The poly-his tagged PRO-C-MG.2, PRO-C-MG.12, PRO-C-MG.45, PRO-C-MG.64 or PRO-C-MG.72 insert can then be subcloned into a SV40 driven vector containing a selection marker such as DHFR for selection of stable clones. Finally, the CHO cells can be transfected (as described above) with the SV40 driven vector. Labeling can be performed, as described above, to verify expression. The culture medium containing the expressed poly-His tagged PRO-C-MG.2, PRO-C-MG.12, PRO-C-MG.45, PRO-C-MG.64 or PRO-C-MG.72 can then be concentrated and purified by any selected method, such as by Ni²-chelate affinity chromatography.

PRO-C-MG.2, PRO-C-MG.12, PRO-C-MG.45, PRO-C-MG.64 or PRO-C-MG.72 can also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁷ cells are frozen in an ampule for further growth and production as described herein.

The ampules containing the plasimid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media can be employed, a production medium described in U.S. Pat. No. 5,122,469, issued Jun. 16, 1992 can actually be used. A 3 L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Coming 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### Example 5

Expression of PRO-C-MG.2, PRO-C-MG.12, PRO-C-MG.45, PRO-C-MG.64 or PRO-C-MG.72 in Yeast

The following method describes recombinant expression of PRO-C-MG.2, PRO-C-MG.12, PRO-C-MG.45, PRO-C-MG.64 or PRO-C-MG.72 in yeast.

First, yeast expression vectors are constructed for intracellular production or secretion of PRO-C-MG.2, PRO-C-MG.12, PRO-C-MG.45, PRO-C-MG.64 or PRO-C-MG.72 from the ADH2/GAPDH promoter. DNA encoding PRO-C-MG.2, PRO-C-MG.12, PRO-C-MG.45, PRO-C-MG.64 or PRO-C-MG.72 and the promoter is inserted into suitable restriction enzyme sites in the selected plasmid to direct intracellular expression of PRO-C-MG.2, PRO-C-MG.12, PRO-C-MG.45, PRO-C-MG.64 or PRO-C-MG.72. For secretion, DNA encoding PRO-C-MG.2, PRO-C-MG.12, PRO-C-MG.45, PRO-C-MG.64 or PRO-C-MG.72 can be cloned into the selected plasmid, together with DNA encoding the ADH2/GAPDH promoter, a mammalian signal peptide, or, for example, a yeast alpha-factor or invertase secretory signal/leader sequence, and linker sequences (if needed) for expression of PRO-C-MG.2, PRO-C-MG.12, PRO-C-MG.45, PRO-C-MG.64 or PRO-C-MG.72.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO-C-MG.2, PRO-C-MG.12, PRO-C-MG.45, PRO-C-MG.64 or PRO-C-MG.72 can subsequently be isolated and purified by removing the yeast cells from the fermentation medium by centrifugation and then concentrating the medium using selected cartridge filters. The concentrate containing PRO-C-MG.2, PRO-C-MG.12, PRO-C-MG.45, PRO-C-MG.64 or PRO-C-MG.72 can further be purified using selected column chromatography resins.

### Example 6

Expression of PRO-C-MG.2, PRO-C-MG.12, PRO-C-MG.45, PRO-C-MG.64 or PRO-C-MG.72 in Baculovirus-Infected Insect Cells

The following method describes recombinant expression of PRO-C-MG.2, PRO-C-MG.12, PRO-C-MG.45, PRO-C-MG.64 or PRO-C-MG.72 in Baculovirus-infected insect cells.

The sequence coding for PRO-C-MG.2, PRO-C-MG.12, PRO-C-MG.45, PRO-C-MG.64 or PRO-C-MG.72 is fused upstream of an epitope tag contained within a baculovirus expression vector. Such epitope tags include poly-his tags and immunoglobulin tags (like Fc regions of IgG). A variety of plasmids can be employed, including plasmids derived from commercially available plasmids such as pVL1393 (Novagen). Briefly, the sequence encoding PRO-C-MG.2, PRO-C-MG.12, PRO-C-MG.45, PRO-C-MG.64 or PRO-C-MG.72 or the desired portion of the coding sequence of PRO-C-MG.2, PRO-C-MG.12, PRO-C-MG.45, PRO-C-MG.64 or PRO-C-MG.72 such as the sequence encoding the extracellular domain of a transmembrane protein or the sequence encoding the mature protein if the protein is extracellular is amplified by PCR with primers complementary to the 5′ and 3′ regions. The 5′ primer can incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are perfomied as described by O'Reilley et al., Baculovirus expression vectors: A Laboratory Manual, Oxford: Oxford University Press (1994).

Expressed poly-his tagged PRO-C-MG.2, PRO-C-MG.12, PRO-C-MG.45, PRO-C-MG.64 or PRO-C-MG.72 can then be purified, for example, by Ni²⁺-chelate affinity chromatography as follows. Extracts are prepared from recombinant virus-infected Sf9 cells as described by Rupert et al., Nature, 362:175-179 (1993). Brielly, Sf9 cells are washed, resuspended in sonication buffer (25 mL Hepes, pH7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM lmidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO-C-MG.2, PRO-C-MG.12, PRO-C-MG.45, PRO-C-MG.64 or PRO-C-MG.72 are pooled and dialyzed against loading buffer.

Alternatively, purification of the IgG tagged (or Fc tagged) PRO-C-MG.2, PRO-C-MG.12, PRO-C-MG.45, PRO-C-MG.64 or PRO-C-MG.72 can be performed using known chromatography techniques, including for instance, Protein A or protein G column chromatography.

### Example 7

Preparation of Antibodies that Bind PRO-C-MG.2, PRO-C-MG.12, PRO-C-MG.45, PRO-C-MG.64 or PRO-C-MG.72

This example illustrates preparation of monoclonal antibodies which can specifically bind PRO-C-MG.2, PRO-C-MG.12, PRO-C-MG.45, PRO-C-MG.64 or PRO-C-MG.72.

Techniques for producing the monoclonal antibodies are known in the art and are described, for instance, in Goding, supra. Immunogens that can be employed include purified PRO-C-MG.2, PRO-C-MG.12, PRO-C-MG.45, PRO-C-MG.64 or PRO-C-MG.72, fusion proteins containing PRO-C-MG.2, PRO-C-MG.12, PRO-C-MG.45, PRO-C-MG.64 or PRO-C-MG.72, and cells expressing recombinant PRO-C-MG.2, PRO-C-MG.12, PRO-C-MG.45, PRO-C-MG.64 or PRO-C-MG.72 on the cell surface. Selection of the immunogen can be made by the skilled artisan without undue experimentation.

Mice, such as Balb/c, are immunized with the PRO-C-MG.2, PRO-C-MG.12, PRO-C-MG.45, PRO-C-MG.64 or PRO-C-MG.72 immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice can also be boosted with additional immunization injections. Serum samples can be periodically obtained from the mice by retro-orbital bleeding for testing in ELISA assays to detect anti-PRO-C-MG.2, PRO-C-MG.12, PRO-C-MG.45, PRO-C-MG.64 or PRO-C-MG.72 antibodies.

After a suitable antibody titer has been detected, the animals “positive” for antibodies can be injected with a final intravenous injection of PRO-C-MG.2, PRO-C-MG.12, PRO-C-MG.45, PRO-C-MG.64 or PRO-C-MG.72. Three to four days later, the mice are sacrificed and the spleen cells are harvested. The spleen cells are then fused (using 35% polyethylene glycol) to a selected murine myeloma cell line such as P3×63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO-C-MG.2, PRO-C-MG.12, PRO-C-MG.45, PRO-C-MG.64 or PRO-C-MG.72. Determination of “positive” hybridoma.cells secreting the desired monoclonal antibodies against PRO-C-MG.2, PRO-C-MG.12, PRO-C-MG.45, PRO-C-MG.64 or PRO-C-MG.72 is within the skill in the art.

The positive hybridoma cells can be injected intraperitoneally into syngeneic Balb/c mice to produce ascites containing the anti-PRO-C-MG.2, PRO-C-MG.12, PRO-C-MG.45, PROC-MG.64 or PRO-C-MG.72 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 8

Purification of PRO-C-MG.2, PRO-C-MG.12, PRO-C-MG.45, PRO-C-MG.64 or PRO-C-MG.72 Polypeptides Using Specific Antibodies

Native or recombinant PRO-C-MG.2, PRO-C-MG.12, PRO-C-MG.45, PRO-C-MG.64 or PRO-C-MG.72 polypeptides can be purified by a variety of standard techniques in the art of protein purification. For example, pro-PRO-C-MG.2, PRO-C-MG.12, PRO-C-MG.45, PRO-C-MG.64 or PRO-C-MG.72 polypeptide, mature PRO-C-MG.2, PRO-C-MG.12, PRO-C-MG.45, PRO-C-MG.64 or PRO-C-MG.72 polypeptide, or pre-PRO-C-MG.2, PRO-C-MG.12, PRO-C-MG.45, PRO-C-MG.64 or PRO-C-MG.72 polypeptide is purified by immunoaffinity chromatography using antibodies specific for the PRO-C-MG.2, PRO-C-MG.12, PRO-C-MG.45, PRO-C-MG.64 or PRO-C-MG.72 polypeptide of interest. In general, an immunoaffinity column is constructed by covalently coupling the anti-PRO-C-MG.2, PRO-C-MG.12, PRO-C-MG.45, PRO-C-MG.64 or PRO-C-MG.72 polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PRO-C-MG.2, PRO-C-MG.12, PRO-C-MG.45, PRO-C-MG.64 or PRO-C-MG.72 polypeptide by preparing a fraction from cells containing PRO-C-MG.2, PRO-C-MG.12, PRO-C-MG.45, PRO-C-MG.64 or PRO-C-MG.72 polypeptide in a soluble form. This preparation is derived by solubilization of the whole cell or of a subcellular fraction obtained via differential centrifugation by the addition of detergent or by other methods well known in the art. Alternatively, soluble PRO-C-MG.2, PRO-C-MG.12, PRO-C-MG.45, PRO-C-MG.64 or PRO-C-MG.72 polypeptide can be secreted in useful quantity into the medium in which the cells are grown, by employing a heterologous secretion signal peptide.

A soluble PRO-C-MG.2, PRO-C-MG.12, PRO-C-MG.45, PRO-C-MG.64 or PRO-C-MG.72 polypeptide-containing preparation is passed over the immunoaffinity column, and the column is washed under conditions that allow the preferentia I absorbance of PRO-C-MG.2, PRO-C-MG.12, PRO-C-MG.45, PRO-C-MG.64 or PRO-C-MG.72 polypeptide (e.g., high ionic strength buffers in the presence of detergent). Then, the column is eluted under conditions that disrupt antibody/PRO-C-MG.2, PRO-C-MG.12, PRO-C-MG.45, PRO-C-MG.64 or PRO-C-MG.72 polypeptide binding (e.g., a low pH buffer such as approximately pH 2-3, or a high concentration of a chaotrope such as urea or thiocyanate ion), and PRO-C-MG.2, PRO-C-MG.12, PRO-C-MG.45, PRO-C-MG.64 or PRO-C-MG.72 polypeptide is collected.

### Example 9

### Drug Screening

This invention is particularly useful for screening compounds by using PRO-C-MG.2, PRO-C-MG.12, PRO-C-MG.45, PRO-C-MG.64 or PRO-C-MG.72 polypeptides or binding fragment thereof in any of a variety of drug screening techniques. The PRO-C-MG.2, PRO-C-MG.12, PRO-C-MG.45, PRO-C-MG.64 or PRO-C-MG.72 polypeptide or fragment employed in such a test can either be free in solution, affixed to a solid support, borne on a cell surface, or located intracellularly. One method of drug screening utilizes eukaryotic or prokaryotic host cells which are stably transformed with recombinant nucleic acids expressing the PRO-C-MG.2, PRO-C-MG.12, PRO-C-MG.45, PRO-C-MG.64 or PRO-C-MG.72 polypeptide or fragment. Drugs are screened against such transformed cells in competitive binding assays. Such cells, either in viable or fixed form, can be used for standard binding assays. One can measure, for example, the formation of complexes between PRO-C-MG.2, PRO-C-MG.12, PRO-C-MG.45, PRO-C-MG.64 or PRO-C-MG.72 polypeptide or a fragment and the agent being tested. Alternatively, one can examine the diminution in complex formation between the PRO-C-MG.2, PRO-C-MG.12, PRO-C-MG.45, PRO-C-MG.64 or PRO-C-MG.72 polypeptide and its target cell or target receptors caused by the agent being tested.

Thus, the present invention provides methods of screening for drugs or any other agents which can affect a PRO-C-MG.2, PRO-C-MG.12, PRO-C-MG.45, PRO-C-MG.64 or PRO-C-MG.72 polypeptide-associated disease or disorder. These methods comprise contacting such an agent with an PRO-C-MG.2, PRO-C-MG.12, PRO-C-MG.45, PRO-C-MG.64 or PRO-C-MG.72 polypeptide or fragment thereof and assaying (1) for the presence of a complex between the agent and the PRO-C-MG.2, PRO-C-MG.12, PRO-C-MG.45, PRO-C-MG.64 or PRO-C-MG.72 polypeptide or fragment, or (ii) for the presence of a complex between the PRO-C-MG.2, PRO-C-MG.12, PRO-C-MG.45, PRO-C-MG.64 or PRO-C-MG.72 polypeptide or fragment and the cell, by methods well known in the art. In such competitive binding assays, the PRO-C-MG.2, PRO-C-MG.12, PRO-C-MG.45, PRO-C-MG.64 or PRO-C-MG.72 polypeptide or fragment is typically labeled. Afier suitable incubation, free PRO-C-MG.2, PRO-C-MG.12, PRO-C-MG.45, PRO-C-MG.64 or PRO-C-MG.72 polypeptide or fragment is separated from that present in bound form, and the amount of free or uncomplexed label is a measure of the ability of the particular agent to bind to PRO-C-MG.2, PRO-C-MG.12, PRO-C-MG.45, PRO-C-MG.64 or PRO-C-MG.72 polypeptide or to interfere with the PRO-C-MG.2, PRO-C-MG.12, PRO-C-MG.45, PRO-C-MG.64 or PRO-C-MG.72 polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such asplastic pins or some other surface. As applied to a PRO-C-MG.2, PRO-C-MG.12, PRO-C-MG.45, PRO-C-MG.64 or PRO-C-MG.72 polypeptide, the peptide test compounds are reacted with PRO-C-MG.2, PRO-C-MG.12, PRO-C-MG.45, PRO-C-MG.64 or PRO-C-MG.72 polypeptide and washed. Bound PRO-C-MG.2, PRO-C-MG.12, PRO-C-MG.45, PRO-C-MG.64 or PRO-C-MG.72 polypeptide is detected by methods well known in the art. Purified PRO-C-MG.2, PRO-C-MG.12, PRO-C-MG.45, PRO-C-MG.64 or PRO-C-MG.72 polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO-C-MG.2, PRO-C-MG.12, PRO-C-MG.45, PRO-C-MG.64 or PRO-C-MG.72 polypeptide specifically compete with a test compound for binding to PRO-C-MG.2, PRO-C-MG.12, PRO-C-MG.45, PRO-C-MG.64 or PRO-C-MG.72 polypeptide or fragments thereof. In this manner, the antibodies can be used to detect the presence of any peptide which shares one or more antigenic determinants with PRO-C-MG.2, PRO-C-MG.12, PRO-C-MG.45, PRO-C-MG.64 or PRO-C-MG.72 polypeptide.

### Example 10

### Rational Drug Design

The goal of rational drug design is to produce structural analogs of biologically active polypeptide of interest (ie., a PRO-C-MG.2, PRO-C-MG.12, PRO-C-MG.45, PRO-C-MG.64 or PRO-C-MG.72 polypeptide) or of small molecules with which they interact, e.g., agonists, antagonists, or inhibitors. Any of these examples can be used to fashion drugs which are more active or stable forms of the PRO-C-MG.2, PRO-C-MG.12, PRO-C-MG.45, PRO-C-MG.64 or PRO-C-MG.72 polypeptide or which enhance or interfere with the function of the PRO-C-MG.2, PRO-C-MG.12, PRO-C-MG.45, PRO-C-MG.64 or PRO-C-MG.72 polypeptide in vivo (cf, Hodgson, Bio/Technology, 9: 19-21 (1991)).

In one approach, the three-dimensional structure of the PRO-C-MG.2, PRO-C-MG.12, PRO-C-MG.45, PRO-C-MG.64 or PRO-C-MG.72 polypeptide, or of an PRO-C-MG.2, PRO-C-MG.12, PRO-C-MG.45, PRO-C-MG.64 or PRO-C-MG.72 polypeptide-inhibitor complex, is determined by x-ray crystallography, by computer modeling or, most typically, by a combination of the two approaches. Both the shape and charges of the PRO-C-MG.2, PRO-C-MG.12, PRO-C-MG.45, PRO-C-MG.64 or PRO-C-MG.72 polypeptide must be ascertained to elucidate the structure and to determine active site(s) of the molecule. Less often, useful information regarding the structure of the PRO-C-MG.2, PRO-C-MG.12, PRO-C-MG.45, PRO-C-MG.64 or PRO-C-MG.72 polypeptide can be gained by modeling based on the structure of homologous proteins. In both cases, relevant structural information is used to design analogous PRO-C-MG.2, PRO-C-MG.12, PRO-C-MG.45, PRO-C-MG.64 or PRO-C-MG.72 polypeptide-like molecules or to identify efficient inhibitors. Useful examples of rational drug design can include molecules which have improved activity or stability as shown by Braxton and Wells, Biochemistry, 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO-C-MG.2, PRO-C-MG.12, PRO-C-MG.45, PRO-C-MG.64 or PRO-C-MG.72 polypeptide can be made available to perform such analytical studies as X-ray crystallography. In addition, knowledge of the PRO-C-MG.2, PRO-C-MG.12, PRO-C-MG.45, PRO-C-MG.64 or PRO-C-MG.72 polypeptide amino acid sequence provided herein will provide guidance to those employing computer modeling techniques in place of or in addition to x-ray crystallography.

### Example 11

### Preparation of Antisense Oligonucleotides

Oligonucleotide Synthesis. Unsubstituted and substituted phosphodiester (P═O) oligonucleotides are synthesized on an automated DNA synthesizer (Applied Biosystems model 380B) using standard phosphoramidite chemistry with oxidation by iodine. Phosphorothioates (P═S) are synthesized as for the phosphodiester oligonucleotides except the standard oxidation bottle is replaced by 0.2 M solution of 3H-1,2-benzodithiole-3-one 1,1-dioxide in acetonitrile for the stepwise thiation of the phosphite linkages. The thiation wait step is increased to 68 sec and is followed by the capping step. After cleavage from the CPG column and deblocking in concentrated ammonium hydroxide at 55° C. (18 hr), the oligonucleotides are purified by precipitating twice with 2.5 volumes of ethanol from a 0.5 M NaCl solution. Phosphinate oligonucleotides are prepared as described in U.S. Pat. No. 5,508,270, herein incorporated by reference. Alkyl phosphonate oligonucleotides are prepared as described in U.S. Pat. No.4,469,863, herein incorporated by reference. 3′-Deoxy-3′-methylene phosphonate oligonucleotides are prepared as described in U.S. Pat. Nos. 5,610,289 or 5,625,050, herein incorporated by reference. Phosphoramidite oligonucleotides are prepared as described in U.S. Pat. No., 5,256,775 or U.S. Pat. No. 5,366,878, herein incorporated by reference. Alkylphosphonothioate oligonucleotides are prepared as described in published PCT applications PCT/US94/00902 and PCT/US93/06976 (published as WO 94/17093 and WO 94/02499, respectively), herein incorporated by reference. 3′-Deoxy-3′-amino phosphoramidate oligonucleotides are prepared as described in U.S. Pat. No. 5,476.925, herein incorporated by reference. Phosphotriester oligonucleotides are prepared as described in U.S. Pat. No. 5,023,243, herein incorporated by reference. Borano phosphate oligonucleotides are prepared as described in U.S. Pat. Nos. 5,130,302 and 5,177,198, both herein incorporated by reference.

Oligonucleoside Synthesis. Methylenemethylimino linked oligonucleosides (MMI linked oligonucleosides), methylenedimethylhydrazo linked oligonucleosides (MDH linked oligonucleosides), and methylenecarbonylamino linked oligonucleosides (amide-3 linked oligonucleosides), and methyleneaminocarbonyl linked oligonucleosides (amide-4 linked oligonucleosides), as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference. Formacetal and thioformacetal linked oligonucleosides are prepared as described in U.S. Pat. Nos. 5,264,562 and 5,264,564, herein incorporated by reference. Ethylene oxide linked oligonucleosides are prepared as described in U.S. Pat. No. 5,223,618, herein incorporated by reference.

PNA Synthesis. Peptide nucleic acids (PNAs) are prepared in accordance with any of the various procedures referred to in Peptide Nucleic Acids (PNA): Synthesis, Properties and Potential Applications, Bioorganic & Medicinal Chemistry, 4:5-23 (1996). They are also prepared in accordance with U.S. Pat. Nos. 5,539,082, 5,700,922, and 5,719,262, herein incorporated by reference.

Synthesis of Chimeric Oligonucleotides. [2′-O-Me]-[2′-deoxy]-[2′-O-Me] Chimeric Phosphorothioate Oligonucleotides: Chimeric oligonucleotides having 2′-O-alkyl phosphorothioate and 2′-deoxy phosphorothioate oligonucleotide segments are synthesized using an Applied Biosystems automated DNA synthesizer Model 380B, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by increasing the wait step after the delivery of tetrazole and base to 600 sec repeated four times for RNA and twice for 2′-O-methyl. The fully protected oligonucleotide is cleaved from the support and the phosphate group is deprotected in 3:1 ammonia/ethanol at room temperature overnight then lyophilized to dryness. Treatment in methanolic ammonia for 24 hrs at room temperature is then done to deprotect all bases and sample is again lyophilized to dryness. The pellet is resuspended in 1 M TBAF in THF for 24 hrs at room temperature to deprotect the 2′ positions. The reaction is then quenched with 1 M TEAA and the sample is then reduced to ½ volume by rotovac before being desalted on a G25 size exclusion column. The oligo recovered is then analyzed spectrophotometrically for yield and for purity by capillary electrophoresis and by mass spectrometry. [2′-O-(2-Methoxyethyl)]-[2′-deoxy]-[2′-O-(Methoxyethyl)] Chimeric Phosphorothioate Oligonucleotides: [2′-O-(2-methoxyethyl)]-[2′-deoxy]-[-2′-O-(methoxyethyl)] chimeric phosphorothioate oligonucleotides are prepared as per the procedure above for the 2′-O-methyl chimeric oligonucleotide, with the substitution of 2′-O-(methoxyethyl) amidites for the 2′-O-methyl amidites. [2′-O-(2-Methoxyethyl)Phosphodiester]-[2′-deoxy Phosphorothioate]-[2′-O-(2-Methoxyethyl) Phosphodiester] Chimeric Oligonucleotides: [2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidization with iodine to generate the phosphodiester internucleotide linkages with the wing portions of the chimeric structures and sulfurization utilizing 3H-1.2 benzodithiole-3-one 1,1 dioxide (Beaucage Reagent) to generate the phosphorotioate internucleotide linkages for the center gap. Other chimeric oligonucleotides, chimieric oligonucleosides and mixed chimieric oligonucleotides/oligonucleosides are synthesized according to U.S. Pat. No. 5,623,065, herein incorporated by reference.

### Example 12

### Corneal Angiogenesis Assay

A corneal angiogenesis assay, using VEGF as an inducer of angiogenesis, can be used to test molecules as an antagonist of PRO-C-MG.2, PRO-C-MG.12, PRO-C-MG.45, PRO-C-MG.64 or PRO-C-MG.72 for anti-angiogenic activity, or as an agonist of PRO-C-MG.2, PRO-C-MG.12, PRO-C-MG.45, PRO-C-MG.64 or PRO-C-MG.72 for enhancing angiogenesis. A 1.5 mm incision is made approximately 1 mm from the center of the cornea of isoflurane-ketamine (60-80 mg/kg) xylazine (10-15 mg/kg) anesthetized Sprague-Dawley rats. Using a curved spatula, the incision is bluntly dissected through the stroma towards the outer canthus of the eye. A Hydron pellet (2×20 mm) containing VEGF (200 ng), sucralfate (100 μg) with or without (control) test molecule, at various amounts, is inserted into the base of the pocket. After surgery, the eyes are coated with gentamicin ointment. Animals are observed at 24-48 hr for the occurrence of nonspecific inflammation and then daily thereafter. At day 6, the animals are euthanized and injected with FITC-dextran to allow for visualization of the vasculature. Corneal whole mounts are made of the enucleated eyes and analyzed for neovascular area using the computer assisted image analysis.

The in vivo anti-angiogenic effects of a PRO-C-MG.2, PRO-C-MG.12, PRO-C-MG.45, PRO-C-MG.64 or PRO-C-MG.72 antagonist are shown using Hydron pellets containing 200 ng recombinant VEGF, with or without various amounts of PRO-C-MG.2, PRO-C-MG.12, PRO-C-MG.45, PRO-C-MG.64 or PRO-C-MG.72 antagonist, implanted into the corneas of Sprague-Dawley rats as described above. In vivo enhancement of angiogenisis can also be studied in this system. Data from this experiment will show that pellets containing the combination PRO-C-MG.2, PRO-C-MG.12, PRO-C-MG.45, PRO-C-MG.64 or PRO-C-MG.72 antagonist and VEGF can produce a significant reduction in vessel length compared to the VEGF only (positive) controls. These in vivo data are consistent with the in vitro results, i.e., antagonism of PRO-C-MG.2, PRO-C-MG.12, PRO-C-MG.45, PRO-C-MG.64 or PRO-C-MG.72 produces strong inhibition of angiogenesis in endothelial tissue. Conversley, agonism of PRO-C-MG.2, PRO-C-MG.12, PRO-C-MG.45, PRO-C-MG.64 or PRO-C-MG.72 produces enhancement of angiogenesis in endothelial tissue.

### Example 13

### Endothelial Proliferation Assay

HUVEC are seeded on collagen-coated 96-well plates at 6,000 cells/cm² in Clonetics EGM supplemented with 10% FBS, 2 mM L-glutamine, 100 U/ml penicillin and 100 U/ml streptomycin and allowed to attach for 4 hr. Medium is then replaced with 1× basal medium consisting of M199 supplemented with 1% FBS, 1×ITS, 2 mM L-glutamine, 50 mg/ml ascorbic acid, 26.5 mM NaHCO₃, 100 U/ml penicillin and 100 U/ml streptomycin supplemented with 40 ng/ml bFGF, 40 ng/ml VEGF and 80 nM PMA. Cells are cultured in above medium in the presence of test drugs or vehicle for 4 hr. Then 5 ml (100 mM) of 5′-bromo-2′-deoxyuride (BrdU) is added in a final volume of 100 ml/well and cells are incubated for another 20 hr. BrdU incorporation is evaluated by an ELISA kit from Boehringer Mannheim (Indianapolis, Ind.).

Data are expressed as the mean±standard error. Statistic analysis is perfomied using one-way ANOVA (INSTAT, Graph Pad Software, Sorrento Valley, Calif.). Multiple comparisons against the control are analyzed using Bonferroni modification of Student's t-test to determine differences between groups. A p value <0.05 is accepted as significant.

This assay can indicate that PRO-C-MG.2, PRO-C-MG.12, PRO-C-MG.45, PRO-C-MG.64 or PRO-C-MG.72 antagonists can repress or inhibit growth factor-induced endothelial cell proliferation. BrdU incorporation assays are performed to detect proliferation of endothelial cells cultured on type I collagen-coated surface in medium containing the growth factors, e.g. VEGF, bFGF and PMA, in the presence of vehicle or PRO-C-MG.2, PRO-C-MG.12, PRO-C-MG.45, PRO-C-MG.64 or PRO-C-MG.72 antagonists. The present invention, therefore, is also directed to a method of inhibiting growth factor induced endothelial cell proliferation by contacting endothelial cells with an effective amount of a PRO-C-MG.2, PRO-C-MG.12, PRO-C-MG.45, PRO-C-MG.64 or PRO-C-MG.72 antagonist. Conditions associated with undesired vascularization and angiogenesis resulting from growth factor induced endothelial cell proliferation can be treated by administering the antagonists in the manner described herein.

PRO-C-MG.2, PRO-C-MG.12, PRO-C-MG.45, PRO-C-MG.64 or PRO-C-MG.72 also regulate gene expression events associated with angiogenesis. Flk/KDR and Flt-1 are two structurally related endothelial cell tyrosine kinase receptors for VEGF. The importance of these two receptors during angiogenesis has been clearly demonstrated by the findings that KDR functions as a transducer to signal endothelial cell proliferation and differentiation and that Flt-1 is a critical survival factor involved in endothelial cell morphogenesis (Fong, G. H. et al., (1995) Nature (London) 376:66-70; Ferrara, N. et al., (1997) Endocr. Rev. 18:4-25; Ilan, N. et al., (1998) J. Cell Sci. 111 :3621-3631). Whether enhancing or blocking PRO-C-MG.2, PRO-C-MG.12, PRO-C-MG.45, PRO-C-MG.64 or PRO-C-MG.72 activity alters Flt-1 and KDR gene expression can be readily determined using real time quantitative RT-PCR, in the system using a mixture of growth factors in HUVEC grown in three dimensional collagen gels. It is also well known that the production of proteases (e.g. plasminogen activators) and their inhibitors (e.g. plasminogen activator inhibitor I, PAI-1) is correlated with endothelial cell degradation of extracellular matrix and migration, the two critical steps of the angiogenic processes. Consequently, the effects of PRO-C-MG.2, PRO-C-MG.12, PRO-C-MG.45, PRO-C-MG.64 or PRO-C-MG.72 agonism or antagonism on gene expression of urokinase type plasminogen (uPA) and PAI-1 in three dimensional collagen gels can be readily determined. Treatment of HUVEC with test drug can either reduced or enhance uPA mRNA at about 4 hr and reduce or enhance PAI-1 gene expression at about 24 hr.

### Example 14

### Stimulation of Endothelial Cell Proliferation

This assay is designed to determine whether PRO-C-MG.2, PRO-C-MG.12, PRO-C-MG.45, PRO-C-MG.64 or PRO-C-MG.72 shows the ability to stimulate adrenal cortical capillary endothelial cell (ACE) growth.

Bovine adrenal cortical capillary endothelial cells (ACE) (from primary culture, maximum of 12-14 passages) are plated in 96-well plates at 500 cells/well per 100 microliter. Assay media included low glucose DMEM, 10% calf serum, 2 mM glutamine, and 1× penicillin/streptomycin/fungizone. Control wells included the following: (1) no ACE cells added; (2) ACE cells alone; (3) ACE cells plus VEGF (5 ng/ml); and (4) ACE cells plus FGF (5 ng/ml). The control or test sample, (in 100 microliter volumes), is then added to the wells (at dilutions of 1%, 0.1% and 0.01%, respectively). The cell cultures are incubated for 6-7 days at 37° C./5% CO₂. After the incubation, the media in the wells is aspirated, and the cells are washed 1× with PBS. An acid phosphatase reaction mixture (100 microliter; 0.1M sodium acetate, pH 5.5, 0.1% Triton X-100, 10 mM p-nitrophenyl phosphate) is then added to each well. After a 2 hour incubation at 37° C., the reaction is stopped by addition of 10 microliters 1N NaOH. Optical density (OD) is measured on a microplate reader at 405 nm.

The activity of PRO-C-MG.2, PRO-C-MG.12, PRO-C-MG.45, PRO-C-MG.64 or PRO-C-MG.72 is calculated as the fold increase in proliferation (as determined by the acid phosphatase activity, OD 405 nm) relative to (1) cell only background, and (2) relative to maximum stimulation by VEGF. VEGF (at 3-10 ng/ml) and FGF (at 1-5 ng/ml) are employed as an activity reference for maximum stimulation. Results of the assay are considered “positive” if the observed stimulation is ≧50% increase over background.

### Example 15

### Inhibition of Vascular Endothelial Growth Factor (VEGF) Stimulated Proliferation of Endothelial Cell Growth

The ability of various PRO-C-MG.2, PRO-C-MG.12, PRO-C-MG.45, PRO-C-MG.64 or PRO-C-MG.72 polypeptides to inhibit VEGF stimulated proliferation of endothelial cells can be tested. Specifically, bovine adrenal cortical capillary endothelial cells (ACE) (from primary culture, maximum of 12-14 passages) are plated in 96-well plates at 500 cells/well per 100 microliter. Assay media include low glucose DMEM, 10% calf serum, 2 mM glutamine, and 1× penicillin/streptomycin/fungizone. Control wells include the following: (1) no ACE cells added; (2) ACE cells alone; (3) ACE cells plus 5 ng/ml FGF; (4) ACE cells plus 3 ng/ml VEGF; (5) ACE cells plus 3 ng/ml VEGF plus 1 ng/ml TGF-beta; and (6) ACE cells plus 3 ng/ml VEGF plus 5 ng/ml LIF. The test samples, poly-his tagged PRO-C-MG.2, PRO-C-MG.12, PRO-C-MG.45, PRO-C-MG.64 or PRO-C-MG.72 polypeptides (in 100 microliter volumes), are then added to the wells (at dilutions of 1%, 0.1% and 0.01%, respectively). The cell cultures are incubated for 6-7 days at 37° C./5% CO₂. After the incubation, the media in the wells is aspirated, and the cells are washed 1× with PBS. An acid phosphatase reaction mixture (100 microliter; 0.1M sodium acetate, pH 5.5, 0.1% Triton X-100, 10 mM p-nitrophenyl phosphate) is then added to each well. After a 2 hour incubation at 37° C., the reaction is stopped by addition of 10 microliters 1N NaOH. Optical density (OD) is measured on a microplate reader at 405 nm.

The activity of PRO-C-MG.2, PRO-C-MG.12, PRO-C-MG.45, PRO-C-MG.64 or PRO-C-MG.72 polypeptides is calculated as the percent inhibition of VEGF (3 ng/ml) stimulated proliferation (as determined by measuring acid phosphatase activity at OD 405 nm) relative to the cells without stimulation. TGF-beta is employed as an activity reference at 1 ng/ml, since TGF-beta blocks 70-90% of VEGF-stimulated ACE cell proliferation. The results are indicative of the utility of the PRO-C-MG.2, PRO-C-MG.12, PRO-C-MG.45, PRO-C-MG.64 or PRO-C-MG.72 polypeptides in cancer therapy and specifically in inhibiting tumor angiogenesis. The results are considered positive if the PRO-C-MG.2, PRO-C-MG.12, PRO-C-MG.45, PRO-C-MG.64 or PRO-C-MG.72 polypeptide exhibits 30% or greater inhibition of VEGF stimulation of endothelial cell growth (relative inhibition 30% or greater).

### Example 16

### Induction of c-fos in Endotlielial Cells

This assay is designed to determine whether PRO-C-MG.2, PRO-C-MG.12, PRO-C-MG.45, PRO-C-MG.64 or PRO-C-MG.72 polypeptides show the ability to induce c-fos in endothelial cells.

Human venous umbilical vein endothelial cells (HUVEC, Cell Systems) in growth media (50% Ham's F12 w/o GHT: low glucose, and 50% DMEM without glycine: with NaHCO3, 1% glutamine, 10 mM HEPES, 10% FBS, 10 ng/ml bFGF) are plated on 96-well microtiter plates at a cell density of 1×104 cells/well. The day after plating, the cells are starved by removing the growth media and treating the cells with 100 μl/well test samples and controls (positive control: growth media; negative control: 10 mM HEPES, 140 mM NaCl, 4% (w/v) mannitol, pH 6.8). The cells are incubated for 30 minutes at 37° C., in 5% CO₂. The samples are removed, and the first part of the bDNA kit protocol (Chiron Diagnostics, cat. #6005-037) is followed, where each capitalized reagent/buffer listed below is available from the kit.

Briefly, the amounts of the TM Lysis Buffer and Probes needed for the tests are calculated based on information provided by the manufacturer. The appropriate amounts of thawed Probes are added to the TM Lysis Buffer. The Capture Hybridization Buffer is warmed to room temperature. The bDNA strips are set up in the metal strip holders, and 100 μl of Capture Hybridization Buffer is added to each b-DNA well needed, followed by incubation for at least 30 minutes. The test plates with the cells are removed from the incubator, and the media is gently removed using the vacuum manifold. 100 μl of Lysis Hybridization Buffer with Probes are quickly pipetted into each well of the microtiter plates. The plates are then incubated at 55° C. for 15 minutes. Upon removal from the incubator, the plates are placed on the vortex mixer with the microtiter adapter head and vortexed on the #2 setting for one minute. 80 μl of the lysate is removed and added to the bDNA wells containing the Capture Hybridization Buffer, and pipetted up and down to mix. The plates are incubated at 53° C. for at least 16 hours.

On the next day, the second part of the bDNA kit protocol is followed. Specifically, the plates are removed from the incubator and placed on the bench to cool for 10 minutes. The volumes of additions needed are calculated based upon information provided by the manufacturer. An Amplifier Working Solution is prepared by making a 1:100 dilution of the Amplifier Concentrate (20 fm/μl) in AL Hybridization Buffer. The hybridization mixture is removed from the plates and washed twice with Wash A. 50 μl of Amplifier Working Solution is added to each well and the wells are incubated at 53° C. for 30 minutes. The plates are then removed from the incubator and allowed to cool for 10 minutes. The Label Probe Working Solution is prepared by making a 1:100 dilution of Label Concentrate (40 pmoles/μl) in AL Hybridization Buffer. After the 10-minute cool-down period, the amplifier hybridization mixture is removed and the plates are washed twice with Wash A. 50 μl of Label Probe Working Solution is added to each well and the wells are incubated at 53° C. for 15 minutes. After cooling for 10 minutes, the Substrate is warmed to room temperature. Upon addition of 3 μl of Substrate Enhancer to each ml of Substrate needed for the assay, the plates are allowed to cool for 10 minutes, the label hybridization mixture is removed, and the plates are washed twice with Wash A and three times with Wash D. 50 μl of the Substrate Solution with Enhancer is added to each well. The plates are incubated for 30 minutes at 37° C. and RLU is read in an appropriate luminometer.

The replicates are averaged and the coefficient of variation is determined. The measure of activity of the fold increase over the negative control (HEPES buffer) value is indicated by chemiluminescence units (RLU). The results are considered positive if the PRO-C-MG.2, PRO-C-MG.12, PRO-C-MG.45, PRO-C-MG.64 or PRO-C-MG.72 polypeptide exhibits at least a two-fold value over the negative control. Typically a negative control=about 1.00 RLU at 1.00% dilution, and a Positive control=about 8.39 RLU at 1.00% dilution.

### Example 17

### Human Venous Endothelial Cell Calcium Flux Assay

This assay is designed to determine whether PRO-C-MG.2, PRO-C-MG.12, PRO-C-MG.45, PRO-C-MG.64 or PRO-C-MG.72 polypeptides show the ability to stimulate calcium flux in human umbilical vein endothelial cells (HUVEC, Cell Systems). Calcium influx is a well documented response upon binding of certain ligands to their receptors. A test compound that results in a positive response in the present Ca influx assay can be said to bind to a specific receptor and activate a biological signaling pathway in human endothelial cells. This could ultimately lead, for example to cell division, inhibition of cell proliferation, endothelial tube formation, cell migration, apoptosis, etc.

Human venous umbilical vein endothelial cells (HUVEC, Cell Systems) in growth media (50:50 without glycine, 1% glutamine, 10 mM Hepes, 10% FBS, 10 ng/ml Bfgf), are plated on 96-well microtiter ViewPlates-96 (Packard Instrument Company Part #6005182) microtiter plates at a cell density of 2×104 cells/well. The day after plating, the cells are washed three times with buffer (HBSS plus 10 mM Hepes), leaving 100 μl/well. Then 100 μl/well of 8 μM Fluo-3 (2×) is added. The cells are incubated for 1.5 hours at 37° C./5% CO₂. After incubation, the cells are then washed 3× with buffer (described above) leaving 100 μl/well. Test drugs are prepared on different 96-well plates at 5× concentration in buffer. The positive control corresponded to 50 μM ionomycin (5×); the negative control corresponded to Protein 32. Cell plate and sample plates are run on a FLIPR (Molecular Devices) machine. The FLIPR machine added 25 μl of test sample to the cells, and readings are taken every second for one minute, then every 3 seconds for the next three minutes.

The fluorescence change from baseline to the maximum rise of the curve (Δ change) is calculated, and replicates averaged. The rate of fluorescence increase is monitored, and only those samples which had a Δ change greater than 1000 and a rise within 60 seconds, are considered positive. Results are expressed relative to the positive control.

### Example 18

### Endothelial Cell Tube Formation Assay

As an alternative to the tube formaiton assay described in Example 1, either of the following tube formation assays can be used. In the tube formation assay, agents that stimulate or inhibit endothelial tube formation, including agents involved in stimulation or inhibiting tracking, chemotaxis, and/or endothelial shape change, in the 3-dimensional matrix, and in particular those that stimulate endothelial cells to differentiate into a tube-like structure in a 3-dimensional matrix in the presence of an exogenous growth factor (e.g., VEGF, bFGF), can be readily identified. These agents can be agonists or antagonists as described herein.

Matrigel Tube Formation Assay. 0.5 ml of Matrigel (Becton Dickinson #4023) is pipeted on to the sureface of each well of a 24 well tissue culture plate. The matrigel is allowed to solidify by incubation at 37° C. for 20 min. Human umbilical vein endothelial cells are resuspended in culture medium (Medium 199, supplemented with 1% fetal bovine serum, 1× insulin-transferrin-selenium (ITS) solution, 2 mmol/L L-glutamine, 26.5 NaHCO₃, 100 U/ml penicillin, 100 U/ml streptomycin) at 2×10⁵ cells per ml. One of the following can be added: (a) no additives; (b) basic fibroblast growth factor (40 ng/ml); (c) vascular endothelial cell growth factor (40 ng/ml); (d) phorbol myristate acetate (80 nM); (e) the combination of (b), (c) and (d); and, anyone or more of (a), (b), (c), (d) or (e) in combination with added test agent at various concentrations. Then 200 ul per well of the cell suspension is added to top of the solidified Matrigel (about 40,000 cells/well). The cells are then cultured in a humidified 5% CO₂ incubator and observed at 4, 8 and 24 hrs. Activity in the assay is measured as an alteration in the formation of tube-like structures, which can be quantitated by measurement of the length of tube-like structures formed and/or the area of the culture covered by the tube-like network.

Fibrin gel tube formation assay. A thrombin solution is prepared by the addition of 2 ml of basal medium (Medium 199 supplemented with 1% fetal bovine serum, 1× insulin-transferrin-selenium (ITS), 2 mmol/L L-glutamine, 26.5 NaHCO₃, 100 U/ml penicillin, 100 U/ml streptomycin) to 100 U thrombin (Sigma Chemical Company, Catalog 6634). This is kept on ice. A fibrinogen solution is prepared by dissolving 112 mg of fibrinogen (Sigma Chemical Company, Catalog #F-4883) in 44 ml of basal medium. Human umbilical endothelial cells are then suspended at a final concentration of 4×10⁵ cells per ml in the fibrinogen solution. The thrombin solution is then aliquoted (10 ul/well) to wells of a 48 well tissue culture plate on ice. Then 300 ul of the HUVEC/Fibrinogen mix is added to each thrombin containing well and mixed by pipetting up and down 3 to 5 times. The plates are incubated at 37° C. for 20 minutes to allow solidification of the fibrin gel. Basal media is then added with one of the following: (a) no additives; (b) basic fibroblast growth factor (40 ng/ml); (c) vascular endothelial cell growth factor (40 ng/ml); (d) phorbol myristate acetate (80 nM); (e) the combination of (b), (c) and (d); and, anyone or more of (a), (b), (c), (d) or (e) in combination with added test agent at various concentrations. Activity in the assay is measured as an alteration in the formation of tube-like structures, which can be quantitated by measurement of the length of tube-like structures formed and/or the area of the culture covered by the tube-like network.

Agents, either agonists or antagonists (e.g., those that block PRO-C-MG.2, PRO-C-MG.12, PRO-C-MG.45, PRO-C-MG.64 or PRO-C-MG.72 expression), can also be quickly screened with the following scoring in which a positive result is equal to or greater than 2: score of 1, cells are all round; score of 2, cells are elongated; score of 3, cells are forming tubes with some connections; and score of 4, cells are forming complex tubular networks.

Optionally, one can add to the cell solutions about 1 μM 6-FAM-FITC dye to stain vacuoles while they are forming. And, after incubation, cells can be fixed with 3.7% formalin at room temperature for 10 minutes, washed with PBS five times, then stained with Rh-Phalloidin at 4° C. overnight followed by nuclear staining with 4 μM DAP1. Subsequently, the cells can be scored for the effect of agent on apoptosis, allowing one to identify factors that facilitate or reduce cell survival in the 3-dimensional matrix, particularly in the presence of exogenous growth factors (e.g., VEGF, bFGF). In this apoptosis assay, a positive result is equal to or less than about 1, where 0=no apoptosis, 1=less than about 20% cells are apoptotic, 2=less than about 50% cells are apoptotic, and 3=greater than about 50% cells are apoptotic. In additon, with the additon of vacuole stain, one can identify factors that stimulate or reduce endothelial vacuole formation and lumen formation in the presence of bFGF or VEGF (40 ng/ml), along with 1 μM 6-FAM-FITC dye to stain vacuoles while they are forming. Cells are incubated at 37° C./5% CO₂ for 48 hr, fixed with 3.7% formalin at room temperature for 10 minutes, washed with PBS five times, then stained with Rh-Phalloidin at 4° C. overnight followed by nuclear staining with 4 μM DAP1. A positive result is equal to or greater than 2: 1=vacuoles present in less than 20% of cells, 2=vacuoles present in 20-50% of cells, 3=vacuoles present in greater than 50% of cells. This assay is designed to identify factors that are involved in stimulating pinocytosis, ion pumping, permeability, and junction formation.

Deposit of Material

The following materials have been deposited with the American Type Culture Collection,10801 University Blvd., Manassas, Va. 20110-2209, USA (ATCC):

| Material | ATCC Dep. No. | Deposit Date |
|---|---|---|
| DNA-C-MG.2-1776 | PTA-799 | Sep. 28, 1999 |
| DNA-C-MG.12-1776 | PTA-798 | Sep. 28, 1999 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, which is intended as a single illustration of certain embodiments of the invention and any constructs that are functionally equivalent are within the scope of this invention. The deposit of material herein does not constitute an admission that the written description herein contained is inadequate to enable the practice of any embodiment of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

1. An isolated nucleic acid molecule that comprises a nucleotide sequence having at least about 80% sequence identity to (a) a nucleotide sequence encoding a PRO-C-MG.2. PRO-C-MG.12, PRO-C-MG.45, PRO-C-MG.64, or PRO-C-MG.72 polypeptide comprising the sequence of amino aid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively, or (b) the complement of the nucleotide sequence of (a).

2. The isolated nucleic acid molecule of claim 1, comprising the nucleotide sequence from about 66 to about 1796 of SEQ ID NO:1, about 465 to about 1886 of SEQ ID O:3, about 271 to about 1788 of SEQ ID NO:17, about 267 to about 1298 of SEQ ID NO:15, or about 71 to about 2059 of SEQ ID NO:13, respectively.

3. The isolated nucleic acid molecule of claim 1, comprising the nucleotide sequence of SEQ ID NO:1 or SEQ ID NO:3, respectively.

4. The isolated nucleic acid molecule of claim 1, comprising a nucleotide sequence that encodes the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively.

5. An isolated nucleic acid molecule comprising a nucleotide sequence that comprises at least about 80% sequence identity to (a) a nucleotide sequence encoding the polypeptide encoded by the human protein cDNA deposited with the ATCC on Sep. 28, 1999, under ATCC Deposit NO PTA-799, on Sep. 28, 1999, under ATCC Deposit No. PTA-798. (DNA-C-MG.2-177 and DNA-C-MG.12-1776, respectively), or (b) the complement of the DNA molecule of (a).

6. The isolated nucleic acid molecule of claim 5, comprising a nucleotide sequence encoding the polypeptide encoded by the human protein cDNA deposited with the ATCC on Sep. 28, 1999, under ATCC Deposit No. PTA-799, on Sep. 29, 1999, under ATCC Deposit No. PTA-798, (DNA-C-MG.2-1776 and DNA-C-MG.12-1776, respectively).

7. An isolated nucleic acid molecule comprising a nucleotide sequence that comprises at least about 80% sequence identity to (a) the full-length polypeptide coding sequence of the human protein cDNA deposited with the ATCC on Sep. 28, 1999, under ATCC Deposit No. PTA-799, on Sep. 28, 1999, under ATCC Deposit No. PTA-798, (DNA-C-MG.2-1776 and, DNA-C-MG.12-1776, respectively), or (b) the complement of the coding sequence of (a).

8. The isolated nucleic acid molecule of claim 7 comprising the full-length polypeptide coding sequence of the human protein cDNA deposited with the ATCC on Sep. 28, 1999, under ATCC Deposit No. PTA-799, on Sep. 28, 1999, under ATCC Deposit No. PTA-798, (DNA-C-MG.2-1776 and DNA-C-MG.12-1776, respectively).

9. An isolated nucleic acid molecule encoding a PRO-C-MG.2, Pro-C-MG.12, Pro-C-MG.45, Pro-C-MG.64 or PRO-C-MG.72 polypeptide comprising nucleic acid that hybridizes to the complement of the nucleic acid sequence that encode amino acids about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively.

10. (canceled)

11. The isolated nucleic acid molecule of claim 9, wherein the hybridization occurs under stringent hybridization or wash conditions.

12-14. (canceled)

15. A vector comprising the nucleic acid molecule of claim 1.

16. The vector of claim 15, wherein the nucleic acid molecule is operably linked to control sequences recognized by a host cell transformed with the vector.

17. (canceled)

18. A host cell comprising the vector of claim 15.

19. The host cell of claim 18, wherein the cell is a CHO cell.

20. The host cell of claim 18, wherein the cell is an E. coli.

21. The host cell of claim 18, wherein the cell is a yeast cell.

22. A process for producing a PRO-C-MG.2, PRO-C-MG.12, PRO-C-MG.45, PRO-C-MG.64, or PRO-C-MG.72 polypeptide comprising culturing the host cell of claim 18 under conditions suitable for expression of the PRO-C-MG.2, PRO-C-MG.12, PRO-C-MG.45, PRO-C-MG.64, or PRO-C-MG.72 polypeptide, wherein the PRO-C-MG.2, PRO-C-MG.12, PRO-C-MG.45, PRO-C-MG.64, or PRO-C-MG.72 polypeptide is produced.

23. The process of claim 22, further comprising the step of recovering the PRO-C-MG.2, PRO-C-MG.12, PRO-C-MG.45, PRO-C-MG.64, or PRO-C-MG.72 polypeptide from the cell culture.

24. An isolated PRO-C-MG.2, PRO-C-MG.12, PRO-C-MG.45, PRO-C-MG.64, or PRO-C-MG.72 polypeptide comprising an amino acid sequence comprising at least about 80% sequence identity to the sequence of amino acid residues from about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively.

25. The isolated PRO-C-MG.2, PRO-C-MG.12, PRO-C-MG.45, PRO-C-MG.64, or PRO-C-MG.72 polypeptide of claim 24 comprising amino acid residues about 1 to about 577 of SEQ ID NO:2, about 1 to about 474 of SEQ ID NO: 4, about 1 to about 506 of SEQ ID NO: 18, about 1 to about 344 of SEQ ID NO: 16, or about 1 to about 633 of SEQ ID NO: 14, respectively.

26. An isolated PRO-C-MG.2, PRO-C-MG.12, PRO-C-MG.45, PRO-C-MG.64, or PRO-C-MG.72 polypeptide having at least about 80% sequence identity to the polypeptide encoded by the cDNA insert of the vector deposited with the ATCC on Sep. 28, 1999, under ATCC Deposit No. PTA-799, on Sep. 28, 1999, under ATCC Deposit No. PTA-798, (DNA-C-MG.2-1776 and DNA-C-MG.12.1776, respectively).

27. The isolated PRO-C-MG.2, or PRO-C-MG.12, polypeptide of claim 26 which is encoded by the cDNA insert of the vector deposited with the ATCC on Sep. 28, 1999, under ATCC Deposit No. PTA-799, on Sep. 28, 1999, under ATCC Deposit No. PTA-798, (DNA-C-MG.2-1776 and DNA-C-MG.12-1776, respectively).

28-31. (canceled)

32. A chimeric molecule comprising a PRO-C-MG.2, PRO-C-MG.12, PRO-C-MG.45, PRO-C-MG.64, or PRO-C-MG.72 polypeptide fused to a heterologous amino acid sequence.

33. The chimeric molecule of claim 32, wherein the heterologous amino acid sequence is an epitope tag sequence.

34. The chimeric molecule of claim 32, wherein the heterologous amino acid sequence is a secretion signal peptide.

35. The chimeric molecule of claim 32, wherein the heterologous amino acid sequence is a Fc region of an immunoglobulin.

36. An antibody which specifically binds to a PRO-C-MG.2, PRO-C-MG.12, PRO-C-MG.45, PRO-C-MG.64, or PRO-C-MG.72 polypeptide.

37. The antibody of claim 36, wherein the antibody is a monoclonal antibody.

38. The antibody of claim 36, wherein the antibody is a humanized antibody.

39. The antibody of claim 36, wherein the antibody is an antibody fragment.

40-59. (canceled)

60. A composition comprising (a) a PRO-C-MG.2, PRO-C-MG.12, PRO-C-MG.45, PRO-C-MG.64 or PRO-C-MG.72 polypeptide, (b) an agonist to a PRO-C-MG.2, PRO-C-MG.12, PRO-C-MG.45, PRO-C-MG.64 or PRO-C-MG.72 polypeptide, (c) an antagonist to a PRO-C-MG.2, PRO-C-MG.12, PRO-C-MG.45, PRO-C-MG.64 or PRO-C-MG.72 polypeptide, or (d) an anti-PRO-C-MG.2, PRO-C-MG.12, PRO-C-MG.45, PRO-C-MG.64 or PRO-C-MG.72 antibody in admixture with a pharmaceutically acceptable carrier.

61-64. (canceled)

65. The composition of claim 60, wherein the antagonist is an PRO-C-MG.2, PRO-C-MG.12, PRO-C-MG.45, PRO-C-MG.64 or PRO-C-MG.72 antisense molecule or antibody.

66-67. (canceled)

68. An article of manufacture comprising:
(a) a composition comprising (i) a PRO-C-MG.2, PRO-C-MG.12, PRO-C-MG.45, PRO-C-MG.64 or PRO-C-MG.72 polypeptide, (ii) an agonist of a PRO-C-MG.2, PRO-C-MG.12, PRO-C-MG.45, PRO-C-MG.64 or PRO-C-MG.72 polypeptide, or (iii) an antagonist of a PRO-C-MG.2, PRO-C-MG.12, PRO-C-MG.45, PRO-C-MG.64 or PRO-C-MG.72 polypeptide, in admixture with a pharmaceutically acceptable carrier;
(b) a container containing the composition; and
(c) a label affixed to said container, or a package insert included in said pharmaceutical product referring to the use of (a) the treatment of an angiogenic disorder.

69. (canceled)

70. The article of manufacture of claim 68, wherein the antagonist is an anti-PRO-C-MG.2, PRO-C-MG.12, PRO-C-MG.45, PRO-C-MG.64 or PRO-C-MG.72 antibody or antigene compound.

71-73. (canceled)

74. A method for identifying a compound that inhibits an activity of a PRO-C-MG.2, PRO-C-MG.12, PRO-C-MG.45, PRO-C-MG.64 or PRO-C-MG.72 polypeptide comprising contacting a test compound with a PRO-C-MG.2, PRO-C-MG.12, PRO-C-MG.45, PRO-C-MG.64 or PRO-C-MG.72 polypeptide under conditions and for a time sufficient to allow the test compound and polypeptide to interact and determining whether the activity of said PRO-C-MG.2, PRO-C-MG.12, PRO-C-MG.45, PRO-C-MG.64 or PRO-C-MG.72 polypeptide is inhibited.

75. A method for identifying a compound that inhibits the expression of a PRO-C-MG.2, PRO-C-MG.12, PRO-C-MG.45, PRO-C-MG.64 or PRO-C-MG.72 polypeptide or gene in cells that normally expresses the polypeptide, wherein the method comprises contacting the cells with a test compound under conditions suitable for allowing expression of said PRO-C-MG.2, PRO-C-MG.12, PRO-C-MG.45, PRO-C-MG.64 or PRO-C-MG.72 polypeptide and determining whether the expression of the PRO-C-MG.2, PRO-C-MG.12, PRO-C-MG.45, PRO-C-MG.64 or PRO-C-MG.72 polypeptide or gene is inhibited.

76-78. (canceled)

79. A method of diagnosing a cardiovascular, endothelial or angiogenic disorder in a mammal which comprises analyzing the level of expression of a gene encoding a PRO-C-MG.2, PRO-C-MG.12, PRO-C-MG.45, PRO-C-MG.64 or PRO-C-MG.72 polypeptide (a) in a test sample of tissue cells obtained from said mammal, and (b) in a control sample of known normal tissue cells of the same cell type, wherein a higher or lower expression level in the test sample as compared to the control sample is indicative of the presence of a cardiovascular, endothelial or angiogenic disorder in said mammal.

80. A method of diagnosing a cardiovascular, endothelial or angiogenic disorder in a mammal which comprises detecting the presence or absence of a PRO-C-MG.2, PRO-C-MG.12, PRO-C-MG.45, PRO-C-MG.64 or PRO-C-MG.72 polypeptide in a test sample of tissue cells obtained from said mammal, wherein the presence or absence of said PRO-C-MG.2, PRO-C-MG.12, PRO-C-MG.45, PRO-C-MG.64 or PRO-C-MG.72 polypeptide in test sample is indicative of the presence of a cardiovascular, endothelial or angiogenic disorder in said mammal.

81. A method of diagnosing a cardiovascular, endothelial or agiogenic disorder in a mammal according to claim 80 comprising (a) contacting an anti-PRO-C-MG.2, PRO-C-MG.12, PRO-C-MG.45, PRO-C-MG.64 or PRO-C-MG.72 antibody with a test sample of tissue cells obtained from the mammal, and (b) detecting the formation of a complex between the anti-PRO-C-MG.2, PRO-C-MG.12, PRO-C-MG.45, PRO-C-MG.64 or PRO-C-MG.72 antibody and a PRO-C-MG.2, PRO-C-MG.12, PRO-C-MG.45, PRO-C-MG.64 or PRO-C-MG.72 polypeptide in the test sample, wherein the formation of said complex is indicative of the presence of a cardiovascular, endothelial or angiogenic disorder in the mammal.

82-84. (canceled)

85. A method for treating a cardiovascular, endothelial or angiogenic disorder in a mammal comprising administering to the mammal a therapeutically effective amount of (a) a PRO-C-MG.2, PRO-C-MG.12, PRO-C-MG.45, PRO-C-MG.64 or PRO-C-MG.72 polypeptide, (b) an agonist of a PRO-C-MG.2, PRO-C-MG.12, PRO-C-MG.45, PRO-C-MG.64 or PRO-C-MG.72 polypeptide, or (c) an antagonist of a PRO-C-MG.2, PRO-C-MG.12, PRO-C-MG.45, PRO-C-MG.64 or PRO-C-MG.72 polypeptide.

86. The method of claim 85, wherein the disorder is vascular trauma or cancer.

87-89. (canceled)

90. The method of claim 85 wherein said antagonist is an anti-PRO-C-MG.2, PRO-C-MG.12, PRO-C-MG.45, PRO-C-MG.64 or PRO-C-MG.72 antibody or antigene.

91. A method for treating a cardiovascular, endothelial or angiogenic disorder in a mammal comprising administering to the mammal a nucleic acid molecule that encodes (a) a PRO-C-MG.2, PRO-C-MG.12, PRO-C-MG.45, PRO-C-MG.64 or PRO-C-MG.72 polypeptide, (b) an agonist of a PRO-C-MG.2, PRO-C-MG.12, PRO-C-MG.45, PRO-C-MG.64 or PRO-C-MG.72 polypeptide, or (c) an antagonist of a PRO-C-MG.2, PRO-C-MG.12, PRO-C-MG.45, PRO-C-MG.64 or PRO-C-MG.72 polypeptide.

92. (canceled)

93. The method of claim 91 wherein said antagonist is an anit-PRO-C-MG.2, PRO-C-MG.12, PRO-C-MG.45, PRO-C-MG.64 or PRO-C-MG.72 antibody or antigene.

94. (canceled)

95. The method of claim 91, wherein the cardiovascular, endothelial or angiogenic disorder is vascular trauma or a cancer.

96-97. (canceled)

98. A method for modulating endothelial cell growth in a mammal comprising administering to the mammal an effective amount of (a) PRO-C-MG.2, PRO-C-MG.12, PRO-C-MG.45, PRO-C-MG.64 or PRO-C-MG.72 polypeptide, (b) an agonist of a PRO-C-MG.2, PRO-C-MG.12, PRO-C-MG.45, PRO-C-MG.64 or PRO-C-MG.72 polypeptide, (c) an antagonist of a PRO-C-MG.2, PRO-C-MG.12, PRO-C-MG.45, PRO-C-MG.64 or PRO-C-MG.72 polypeptide, or (d) an anti-PRO-C-MG.2, PRO-C-MG.12, PRO-C-MG.45, PRO-C-MG.64 or PRO-C-MG.72 antibody, wherein endothelial cell growth in said mammal is inhibited.

99. (canceled)

100. A method for modulating angiogenesis comprising administering an effective amount of an (a) a PRO-C-MG.2, PRO-C-MG.12, PRO-C-MG.45, PRO-C-MG.64 or PRO-C-MG.72 polypeptide, (b) an agonist of a PRO-C-MG.2, PRO-C-MG.12, PRO-C-MG.45, PRO-C-MG.64 or PRO-C-MG.72 polypeptide, or (c) an antagonist of a PRO-C-MG.2, PRO-C-MG.12, PRO-C-MG.45, PRO-C-MG.64 or PRO-C-MG.72 polypeptide, to the mammal, wherein said angiogenesis is inhibited.

101-103. (canceled)

104. A method for treating a tumor, reducing the size of a tumor, reducing the vasculature supporting a tumor, or reducing the tumor burden of a mammal, comprising administering to a mammal in need thereof a therapeutically effective amount of (a) a PRO-C-MG.2, PRO-C-MG.12, PRO-C-MG.45, PRO-C-MG.64 or PRO-C-MG.72 polypeptide, (b) an agonist of a PRO-C-MG.2, PRO-C-MG.12, PRO-C-MG.45, PRO-C-MG.64 or PRO-C-MG.72 polypeptide, or (c) an antagonist of a PRO-C-MG.2, PRO-C-MG.12, PRO-C-MG.45, PRO-C-MG.64 or PRO-C-MG.72 polypeptide.

105. A method for treating a disease or disorder characterized by undesirable excessive neovascularization, comprising administering to a mammal in need thereof a therapeutically effective amount of (a) a PRO-C-MG.2, PRO-C-MG.12, PRO-C-MG.45, PRO-C-MG.64 or PRO-C-MG.72 polypeptide, (b) an agonist of a PRO-C-MG.2, PRO-C-MG.12, PRO-C-MG.45, PRO-C-MG.64 or PRO-C-MG.72 polypeptide, or (c) an antagonist of a PRO-C-MG.2, PRO-C-MG.12, PRO-C-MG.45, PRO-C-MG.64 or PRO-C-MG.72 polypeptide.

106. The method of claim 105, wherein the disease or disorder is selected from the group consisting of rheumatoid arthritis, psoriasis, atherosclerosis, retinopathy, retrolental fibroplasias, neovascular glaucoma, age-related macular degeneration, thyroid hyperplasias, Grave's disease, tissue transplantation, chronic inflammation, lung inflammation, and obesity.

107-112. (canceled)


SEQUENCE LISTING (29 sequences)

SEQ ID NO: 1 - DNA, 2891 bp, Homo sapiens
ggcgggagtcggcgcctcgggttcctacctcgcgtccctaggcggcggcggccgggcgtcccgggatggccttcatggagaagccgccagccggcaaggtgctgctggacgacacggtgccgctgacagcagccatcgaggcgagccagagcctgcagtcccacacggaatatattattcgagtgcaaagaggaatttctgtggaaaacagctggcagattgttagaagatacagtgactttgatttgcttaacaacagcttacagattgcaggcctaagtctacctcttcctcccaaaaaattgattggtaacatggatcgtgaattcatagctgaaaggcagaaaggtcttcagaactatctcaacgtgatcacaacaaatcatatcttgtctaattgtgagctggttaagaagtttttagatccaaacaactattccgcaaactatactgagattgccttgcaacaggtttccatgttcttccgatcagaaccaaagtgggaggtggtggaacctttgaaagacataggttggagaataaggaagaaatatttcttgatgaagattaaaaatcagccaaaggaacggctagtgttaagctgggctgaccttggcccagacaagtatttgtcagataaagattttcagtgtctaatcaaacttctgccttcttgtttgcacccttacatctatcgggttacctttgccacagctaatgaatcctcagcgttgctaattaggatgtttaacgaaaagggaacattgaaggatctgatctacaaggcaaaaccaaaagacccatttctaaagaagtactgcaaccctaagaagattcagggcctggaactccagcaaataaaaacatatggacggcaaatattagaggtactgaagtttcttcatgacaagggattcccttatgggcatcttcacgcctccaatgtgatgctcgatggggacacttgtcggctgctggaccttgagaattccttattgggcctgccttccttctaccgatcttatttttcacaattcaggaaaatcaatacattggaaagtgtggatgtccactgctttggccacttactgtatgaaatgacttatggacgaccgccagactcggtgcctgtggactccttccctcctgccccgtccatggctgtggtggccgtgttggagtctacgctgtcttgtgaagcctgtaaaaatggcatgcctaccatctcccggctcttacagatgccattattcagcgatgttttactaaccacttctgaaaaaccacagtttaagatccctacaaagttaaaagaggcattgagaattgccaaagaatgtatagagaagagactaattgaggaacagaaacagattcaccagcatcgaagactgacaagagctcagtcccaccatggatctgaggaggaaagaaaaaaaagaaagattttagctcgaaagaagtcaaaacgatctgctcttgaaaatagtgaagagcattcagcgaggtacagcaactccaataattcaggatctggggccagctcacctctcacgtccccgtcatcgccaactccaccctctacatcagggatatctgcattacctccacctcctccacctccaccaccaccagcagctcccttgcctcctgcgagcaccgaggtacctgcccagctctcgtctcaggctgtgaatggcatgagccgaggggccttgctcagctccatccagaatttccaaaaaggaactttgaggaaagccaaaacctgtgatcacagtgctccgaagatcggctgaagcttcctgtttacacttggagggaaaagttcttttttattcctactcacccctaccccccaaactaccctcttcctgggaaagtaattgctgagccagtacagccacaaacagtactattttgcagatgctcatgtaagcagcttttcgagagaaataattctttaagcagaataaagttaggctggcattgctcccttaagatcttgctcctttattaaccctgtaaaggagtcttgtttatcctctaatggccaggcttttgggacagcagcatattgaaatattttcaccaactaaaggaaatagacagaaaaacaatgacaatattcaatcacagcagtaaatggcctttgtgttgcaatcccttctaccccatcagacagctcctagaaacattccttacagttcatttctctaaagcattttctgattcttagataactccaatttttgctacctttatcttagacattaacactatagcccaaagcatagttactttgctaaatcagaaagcaactgagttctttgttttctcctcaaatagaatggggaacgttcacaacattctcttaagttctaacaggaataccattgtggttatagaactcagggctgctaaagcaactactctagacccatagttctttttagttagatgtattgaaacagacaaaaatattaacatcagaaaaagctcttgccaattagaggatcttcttaatcctcagcaattaagtttggggtttgaggggggcaggtcattgttacaacagaagtaaatttggcatctatagaaatcaattatgatttttgaaagatttatctaaatatatcaatatagcatctctttaatgttagtcatttattagaaagatcctttatcctgatttgcttaaacctttcaataaattgcactttaaaggattataaataatccatttaaaaattcaagtacacacatcagtgttggttactatgcagagaatgtcattgtgtatagtttcatgtaatctgttatgtcagctgtattttttattaaaatcatgtcaagaaaaaaaaaaaaaaaaaaaaaaaaaaaaaaaaaaa

SEQ ID NO: 2 - protein, 577 aa, Homo sapiens
MAFMEKPPAGKVLLDDTVPLTAAIEASQSLQSHTEYIIRVQRGISVENSWQIVRRYSDFDLLNNSLQIAGLSLPLPPKKLIGNMDREFIAERQKGLQNYLNVITTNHILSNCELVKKFLDPNNYSANYTEIALQQVSMFFRSEPKWEVVEPLKDIGWRIRKKYFLMKIKNQPKERLVLSWADLGPDKYLSDKDFQCLIKLLPSCLHPYIYRVTFATANESSALLIRMFNEKGTLKDLIYKAKPKDPFLKKYCNPKKIQGLELQQIKTYGRQILEVLKFLHDKGFPYGHLHASNVMLDGDTCRLLDLENSLLGLPSFYRSYFSQFRKINTLESVDVHCFGHLLYEMTYGRPPDSVPVDSFPPAPSMAVVAVLESTLSCEACKNGMPTISRLLQMPLFSDVLLTTSEKPQFKIPTKLKEALRIAKECIEKRLIEEQKQIHQHRRLTRAQSHHGSEEERKKRKILARKKSKRSALENSEEHSARYSNSNNSGSGASSPLTSPSSPTPPSTSGISALPPPPPPPPPPAAPLPPASTEVPAQLSSQAVNGMSRGALLSSIQNFQKGTLRKAKTCDHSAPKIG

SEQ ID NO: 3 - DNA, 2119 bp, Homo sapiens
tgcacctcggttctatcgattcgaattcggccacactggccggatcctctagagatccctcgacctcgacccacgcgtccgctcaaggaagttgaatgaggtgaacatagatacagctctttccccttccccccctttttaaatgtaacaaatactttttatgttccccttccccccttccccttttcccctttccccttttggaaacgtgtcaggaaccaaatagtttaagatgagcagttgaggggactgagagagtgatcgacacagaacctggcttcttcgtgcttcatcataagtcgtgctgccggccaggttacttaagcacccttttaacaaggaaaccttgtgggagatccagctggccgactcgagttcagaaacaggaccacagaggttacactctgggatcctggccatgaggttggatgcctcaccttactgaaaggagacactggacctaaatggcgcagcatgattttgttcctgcttggctaaatttctcaacaccacagtcagctaagtcacctactgccaccttcgaaaaacacggagagcacctacccagaggagaaggtagatttggagtaagccgccgtcgacataattcctctgatggtttttttaacaatggtcccctacgaactgcaggagattcttggcaccagccctcctgttccgccatgattctgtggactctggtgtctctaagggagcatatgctggaatcacagggaacccatctggttggcatagctcttccccgaggtcatgatggcatgagccaacgtagtgtaggtggcacagggaaccatcgccattggaatggcagcttccactcccggaaagggtgtgcttttcaggaaaagccacctatggagattagggaagaaaagaaagaagacaaggtggaaaagttgcagtttgaagaggaggactttccttccttgaatccagaagctggcaaacagcatcagccatgcagacctattgggacaccttctggagtatgggaaaacccgcctagtgccaagcaaccctccaagatgctagttatcaaaaaagtttccaaagaggatcctgctgctgccttctctgctgcattcacctcaccaggatctcaccatgcaaatgggaacaaattgtcatccgtggttccaagtgtctataagaacctggttcctaagcctgtaccacctccttccaagcctaatgcatggaaagctaacaggatggagcacaagtcaggatccctttcctctagccgggagtctgcttttaccagtccaatctccgttaccaaaccagtggtactggctagtggtgcagctctgagttctcccaaagagagtccctccagcaccacccctccaattgagatcagctcctctcgtctgaccaagttgacccgccgaaccaccgacaggaagagtgagttcctgaaaactctgaaggatgaccggaatggagacttctcagagaatagagactgtgacaagctggaagatttggaggacaacagcacacctgaaccaaaggaaaatggggaggaaggctgtcatcaaaatggtcttgccctccctgtagtggaagaaggggaggttctctcacactctctagaagcagagcacaggttattgaaagctatgggttggcaggaatatcctgaaaatgatgagaattgccttcccctcacagaggatgagctcaaagagttccacatgaagacagagcagctgagaagaaatggctttggaaagaatggcttcttgcagagccgcagttccagtctgttctccccttggagaagcacttgcaaagcagagtttgaggactcagacaccgaaaccagtagcagtgaaacatcagatgacgatgcctggaagtaggcatataaatgctcacagttaaatctgacccagtaaactctgtgtgtttagggagtatacaaaagaaatcgttcttttccttttcttatgttgttgaatacttcattcacaagggaaataatcatatcccaaagagagaaaaaaaaaaaaaaaagggcggccgcgactctagagtcgacctgcagtagggataacagggtaataagcttggccgccatggcccaacttgt

SEQ ID NO: 4 - protein, 474 aa, Homo sapiens
MAQHDFVPAWLNFSTPQSAKSPTATFEKHGEHLPRGEGRFGVSRRRHNSSDGFFNNGPLRTAGDSWHQPSCSAMILWTLVSLREHMLESQGTHLVGIALPRGHDGMSQRSVGGTGNHRHWNGSFHSRKGCAFQEKPPMEIREEKKEDKVEKLQFEEEDFPSLNPEAGKQHQPCRPIGTPSGVWENPPSAKQPSKMLVIKKVSKEDPAAAFSAAFTSPGSHHANGNKLSSVVPSVYKNLVPKPVPPPSKPNAWKANRMEHKSGSLSSSRESAFTSPISVTKPVVLASGAALSSPKESPSSTTPPIEISSSRLTKLTRRTTDRKSEFLKTLKDDRNGDFSENRDCDKLEDLEDNSTPEPKENGEEGCHQNGLALPVVEEGEVLSHSLEAEHRLLKAMGWQEYPENDENCLPLTEDELKEFHMKTEQLRRNGFGKNGFLQSRSSSLFSPWRSTCKAEFEDSDTETSSSETSDDDAWK

SEQ ID NO: 5 - DNA, 294 bp, Homo sapiens
  unsure 162, 165, 183-184, 187, 204, 207, 211, 216, 219,222-223, 225,: unknown base
cccgggatggccttcatggagaagccgccagctggcaaggtgctgctggacgacacggtgccgctgacagcagccatcgaggcgagccagagcctgcagtcccacacggaatatattattcgagtgcaaagaggaatttctctggaaaacagctggcagatngtnagaagatacagtgacttnnatntgcttaacaacagcttncanattncaggnctnagnntncctcttcctccnaanaaantgattnggnaacatggancgtgnanttcatngctnganaggcagnnaggt

SEQ ID NO: 6 - DNA, 24 bp, Homo sapiens
ggacgacacggtgccgctgacagc

SEQ ID NO: 7 - DNA, 51 bp, Homo sapiens
gccatcgaggcgagccagagcctgcagtcccacacggaatatattattcga

SEQ ID NO: 8 - DNA, 31 bp, Homo sapiens
gttttccagagaaattcctctttgcactcga

SEQ ID NO: 9 - DNA, 793 bp, Homo sapiens
  unsure 379, 433, 459, 492, 517, 541, 549, 561, 575, 579, 582,710, 742,: unknown base
gggaagaaaagaaagaagacaaggtggaaaagttgcagatttgaagaggaggactttccttccttgaatccagaagctggcaaacagcatcagccatgcagacctattgggacaccttctggagtatgggaaaacccgcctagtgccaagcaaccctccaagatgctagttatcaaaaaagtttccaaagaggatcctgctgctgcyttctctgctgcattcacctcaccaggatctcaccatgcaaatgggaacaaattgtcatccgtggttccaagtgtctataagaacctggttcctaagcctgtaccacctccttccaagcctaatgcatggaaagctaacaggatggagcacaagtcaggatccctttcctctnagccgggagtctgcttttaccagtccaatctctgttaccaaaccagtggtactnggctagtggtgcagctctgagttctncccaaagagagtccctccagcaccacccctccnaattgagatcagctcctctcgtctngaccaagtttgacccgccgaaccnaccgacanggaagragtgangttcctgaaaactnctgnaanggatgaccgggaatgggaagactttytcagaagaatagagactgtgacaagctggaagatttggaggacaacagcacactgaaccaaaggaaaatgggggaggaaggctgttcatcaaatggtctttnccttcctgtagttggaaggaggggaggttttntcacatttttaggagcagagcacaggtttttnaagttttggntggcggatt

SEQ ID NO: 10 - DNA, 24 bp, Homo sapiens
gacctattgggacaccttctggag

SEQ ID NO: 11 - DNA, 43 bp, Homo sapiens
cccgcctagtgccaagcaaccctccaagatgctagttatcaaa

SEQ ID NO: 12 - DNA, 25 bp, Homo sapiens
cttggtcagacgagaggagctgatc

SEQ ID NO: 13 - DNA, 2366 bp, Homo sapiens
  unsure 103: unknown base
tggccggatcctctagagatccctcgacctcgacccacgcgtccgcagaaagtatgtcccttctctcaccatgagctggctctccagttcccagggagtggtnctaacagcctaccaccccagcggcaaggaccagaccgtcgggaacagccatgcaaaggcaggggaggaagccacctcgagtcgcagatatggccagtacactatgaaccaggaaagcaccaccatcaaagttatggagaagcctccatttgatcgatcaatttcccaggattctttggatgaactatctatggaagactattggatagaactagaaaacatcaagaaatctagtgaaaacagccaagaagatcaagaggtggttgttgtcaaagagcctgatgagggagaattggaagaagagtggcttaaagaggccggtttatccaatctcttcggagagtctgctggagatccacaggaaagcattgtgtttttatcaacattgacgcggacccaggcagcagcagttcagaagcgagtagagacggtctcccagaccttgaggaaaaaaaacaaacagtaccagattcctgacgtcagagacatatttgctcaacagagagaatcaaaagaaacagctccaggtggcactgaatcgcagtcacttagaacaaatgaaaacaaataccaaggaagagatgacgaggcatctaaccttgttggtgaagagaagctgatcccacctgaggagacgcctgcccctgaaacagacatcaacctggaggtatcatttgccgagcaagcactcaatcagaaagagagatccaaggagaaaatccagaagagcaaaggcgatgatgccacattacctagtttcagattgccaaaagacaaaacgggtaccacaaggattggtgacctcgcaccccaggacatgaagaaagtttgccatttagccctaattgagctgactgccctctatgatgtattgggtattgagctgaaacaacaaaaagctgtgaaaatcaaaacaaaagattctggtcttttttgcgttccattgacagcgctattagaacaagatcagaggaaagtaccaggaatgcgaatacccttgatctttcaaaaactgatttctcgaattgaagagagaggtttggaaacagaaggcctcttacggatccctggagctgccattagaatcaagaatctttgccaagaactagaagcaaagttttatgaagggacttttaattgggaaagtgtcaaacagcatgatgccgccagcctgctgaagctcttcattcgggagttgccccagccactgctcagtgtggagtatctcaaagcctttcaggctgtccagaatcttccaaccaagaagcagcaactacaggctttgaaccttcttgtcatcctcctacctgatgcaaacagggacacactgaaggcccttcttgaatttctccaaagagtaatagataataaagaaaaaaataaaatgacagtcatgaatgtagcaatggtcatggccccgaatctctttatgtgtcatgcattgggattgaagtccagtgaacagcgagaatttgtaatggcagctgggacagcaaataccatgcacttattgattaagtaccaaaaacttctgtggacaattcccaagtttattgtaaaccaagtgaggaagcaaaacacggaaaatcataaaaaggataaaagagccatgaagaaattgctgaagaaaatggcttatgaccgagaaaaatatgaaaagcaagataagagtacaaatgatgctgacgttcctcagggagtgattcgagtgcaagctccccatctttcgaaagtttccatggcaatacagctaactgaagaactaaaagccagtgatgtacttgccaggtttctcagccaagaaagtggggttgcccagactctcaagaaaggagaagtttttttgtatgaaattggaggaaatattggggaacgctgccttgatgatgacacttacatgaaggatttatatcagcttaacccaaatgctgagtgggttataaagtcaaagccattgtagaagacttaacaagctgcagataaccatgtggacttctgtcataattcttgctgagtcaagagtgtaaataaaagaaatggcaggactcatattattcagttgtacccaagtattttaaaaatgactctcttaagccttaaaaagtcatagatttgtgctgctgccagaattatattaaattattattaatggtattattagaaaaaaaaatttctggagtgagagtaaagaggcttaattagtttgtgggcagttttcttatgctctgtgaaatgtgtccagatgtgacatagttttttttttt

SEQ ID NO: 14 - protein, 663 aa, Homo sapiens
MSWLSSSQGVVLTAYHPSGKDQTVGNSHAKAGEEATSSRRYGQYTMNQESTTIKVMEKPPFDRSISQDSLDELSMEDYWIELENIKKSSENSQEDQEVVVVKEPDEGELEEEWLKEAGLSNLFGESAGDPQESIVFLSTLTRTQAAAVQKRVETVSQTLRKKNKQYQIPDVRDIFAQQRESKETAPGGTESQSLRTNENKYQGRDDEASNLVGEEKLIPPEETPAPETDINLEVSFAEQALNQKERSKEKIQKSKGDDATLPSFRLPKDKTGTTRIGDLAPQDMKKVCHLALIELTALYDVLGIELKQQKAVKIKTKDSGLFCVPLTALLEQDQRKVPGMRIPLIFQKLISRIEERGLETEGLLRIPGAAIRIKNLCQELEAKFYEGTFNWESVKQHDAASLLKLFIRELPQPLLSVEYLKAFQAVQNLPTKKQQLQALNLLVILLPDANRDTLKALLEFLQRVIDNKEKNKMTVMNVAMVMAPNLFMCHALGLKSSEQREFVMAAGTANTMHLLIKYQKLLWTIPKFIVNQVRKQNTENHKKDKRAMKKLLKKMAYDREKYEKQDKSTNDADVPQGVIRVQAPHLSKVSMAIQLTEELKASDVLARFLSQESGVAQTLKKGEVFLYEIGGNIGERCLDDDTYMKDLYQLNPNAEWVIKSKPL

SEQ ID NO: 15 - DNA, 1327 bp, Homo sapiens
  unsure 74, 1306: unknown base
gcggggctgcccctgcagcgacagccccatcctgcagccaccaatggcatgacccagggccccggcactgcctngtgtgaggggctggcagctttccaactgcagcaagtggaggcccctgccagcttcgggcctgtgggcaggggctcagtggggcaggggtgtggctgccccgcccggcacgcctgcacctgtctcctctttgtgaccagtaccgcaaggggatcatctcgggctccgtctgccaggacctgtgtgagctgcatatggtggagtggaggacctgcctctcggtggccccgggccagcaggtgtacagcgggctctggcgggacaaggatgtaaccatcaagtgtggcattgaggagaccctcgactccaaggcccggtcggatgcggccccccggcgggagctggtactgtttgacaagcccacccggggcacctccatcaaggaattccgggagatgaccctcggcttcctcaaggcgaacctgggagacctgccttccctgccggcgctggttggccaggtcctgctcatggctgacttcaacaaggacaaccgggtgtccctggcggaagccaagtccgtgtgggccctgctgcagcgtaacgagttcctgctgctgctgtccctgcaggagaaggagcacgcctccagactgctgggctactgtggggacctctacctcaccgagggcgtgccgcatggcgcctggcacgcggccgccctcccacccctgttgcgcccactgctgccgcctgccctgcagggtgctctccagcagtggctggggcctgcgtggccttggcgggccaagatcgccatcggcctgctggagttcgtggaggagctcttccacggctcttacgggactttctacatgtgtgagaccacactggccaacgtgggctacacagccacctacgacttcaagatggccgacctgcagcaggtggcacccgaggccaccgtgcgccgcttcctgcagggccgccgctgcgagcacagcaccgactgcacctacgggcgcgactgcagggccccgtgtgacaggctcatgaggcagtgcaagggcgacctcatccagcccaacctggccaaggtgtgcgcactgctacggggctacctgctgcctggcgcgcccgccgacctccgcgaggagctgggcacacagctgcgcacctgtaccacgctgagcgggctggccagccaggtggaggcccatcactcgctggtgctcagccacctcaagactctgctctggaagaagatctccaacaccaagtactcttgatgggnaatgagggcttgcaaccttct

SEQ ID NO: 16 - protein, 344 aa, Homo sapiens
  unsure 104: unknown amino acid
MVEWRTCLSVAPGQQVYSGLWRDKDVTIKCGIEETLDSKARSDAAPRRELVLFDKPTRGTSIKEFREMTLGFLKANLGDLPSLPALVGQVLLMADFNKDNRVSXAEAKSVWALLQRNEFLLLLSLQEKEHASRLLGYCGDLYLTEGVPHGAWHAAALPPLLRPLLPPALQGALQQWLGPAWPWRAKIAIGLLEFVEELFHGSYGTFYMCETTLANVGYTATYDFKMADLQQVAPEATVRRFLQGRRCEHSTDCTYGRDCRAPCDRLMRQCKGDLIQPNLAKVCALLRGYLLPGAPADLREELGTQLRTCTTLSGLASQVEAHHSLVLSHLKTLLWKKISNTKYS

SEQ ID NO: 17 - DNA, 3020 bp, Homo sapiens
  unsure 4, 107, 109, 112, 119, 122, 176, 323, 1616, 1878, 1881,1911,: unknown base
gcgnccgccgcctctggccgccgccatcgccgcttcagacttcagaaccagcgtcgtcctcaaccacctggccgagaagtggtttccgggccagcgcgagtgctctngngcncggcaantcnggggagctactgcaccaggggcgctaccgggagccctggcgccgcctgcgaggnctgcgagcagaacccagtgacttgattgtaaaaatttacagagcggaatcatatgctggtctccaagagtttaaagcagccttagaagatttaaatgcagttctttttcaacttccaagattggcctgaaggtctacttcaggaaanggaaaaagtactctgcgatgctggttttttaggtgatgccttacaactctttcttcagtgcttagcccttgatgaagattttgcacctgcaaagctgcaagtacaaaagattttatgtgatttattattacctgaaaacttaaaagaaggcctgaaggaatcttcctggagttcattaccatgtactaaaaacagaccttttgattttcattcagtgatggaagagtctcagtctctcaatgaacctagcccaaagcagagtgaagaaataccagaggtcacttcagagcctgtcaaaggaagcttaaaccgtgctcagtcagcacagtctataaattcaacagaaatgcctgccagagaggactgtttaaaaaaagtgtcctcagaacctgttctgtcagttcaagaaaaaggtgttctgctgaaaagaaagttgtctcttttagaacaggatgtgattgtaaatgaagatggaagaaataagctgaaaaaacaaggagaaactcccaatgaagtctgtatgttttccttagcttatggtgatattccagaagaattaatcgatgtctcagatttcgagtgttctctctgcatgaggttgttttttgagccagtaacaaccccttgcggacattcgttctgtaagaattgtcttgagcgttgtttagatcatgcaccatattgtcctctttgcaaagaaagcttaaatgagtatctagcagataggaggtactgtgtcacacagctgttggaagaattaatagtgaagtatctgcctgatgaactgtctgagagaaaaaaaatatatgaagaagaaactgctgaactctcacacttgaccaagaatgttccaatatttgtttgcactatggcctaccccactgtgccttgccctctccatgtatttgagccaagatacagattgatgattcgaagaagtatacagactggaaccaaacagtttggcatgtgtgtcagtgatacacaaaatagttttgcagattatggttgtatgttacaaattagaaacgtgcatttcttaccggacggaaggtctgtggttgatacagttggaggaaagcggtttagggttttaaaaagaggaatgaaagatggatattgcactgccgacattgaatatctggaagatgttaaggttgagaatgaagatgagattaagaatctcagagagcttcatgatttggtttactctcaagcctgcagctggtttcagaatttaagagacagatttcgaagccaaattcttcagcatttcggatcaatgcccgaganggaggaaaaccttcaggcagcccctaatggacctgcatggtgttggtggcttcttgcagttctccctgtagacccacgataccagctgtcggttttgtcaatgaagtctttgaaagaacggttgaccaagatacagcatatactgacctatttttctagagaccaatctaagtaactaactctttggatctccctttaaagtgaccctaatctggctgcattgatggccagattgtctgctgcctttgcacatctagtgctnggntttcagaaatttaatgaaacttttcttttntccttcgacctcnctgaatcatgtggtntctgcaaatgaatnaccttcaactaggatttagaccactaagnaacttgcanncagaaaanacacgcattgaatgtgtgtcgaacctctacattgtgaagttgcactatgtaccatactctaaaatgaaataagaactctttatgtctgtgagagagtgtgtgtgtgtgtgtgcgtgcgtgtgtgcttgtgggggttgggtagtgtgtgtgtattttctctggctttaaaattttaaaacaaacaaaccaaaaagccatagagagcagaacttgccgagggtcatttattgcccaagtttacaagagtagcgatacaagtttttgcaaattgaatttgcctcagatatatctgtcctaatgcttatatttgcacaagtatgtaaaatatcgtgttgaggatcattctttgttggaaatactgctcttgctgaactgtcttgaccattgactatgacacagtttcttatttatgtaaatacttgcatcacagtggccgacagatntgggatgcagaacctagagccagttttcaggaacaattgtaaacctgacatggtactgtgcatctattcataaaacactcaaaactgtgaaaatatggtttacatttaattgtacataaaggtaaagggagaactcaattcagtaccagttagtttgtacattttagggggcttttcacattaactgcccatttgtgtaatttatagtttgacatgatgtgtttgttttaaaaaaaaatgcatagtataaacccattaaggatctgggaaaagagaagaagtttaatatagaactaagcttttaaagtttgtttttgtttttaattctggtctcggtgcaaatgttagttatgccttattcatatcacagttagatcaccatgctgcaacatggtttatattcatgctgccctagaaacttttgtaattatttgttgcaaatttgtgactgtccttattaactttcttttatgtaagtaatttgtaaaagtttcttaaaatttttgcttttgcttatttaattttgaataaaagctaaattcctaataaaaaaaaaaaaaaaaaaaaaaaaaaaaaa

SEQ ID NO: 18 - protein, 506 aa, Homo sapiens
  unsure 18, 448: unknown amino acid
MQFFFNFQDWPEGLLQEXEKVLCDAGFLGDALQLFLQCLALDEDFAPAKLQVQKILCDLLLPENLKEGLKESSWSSLPCTKNRPFDFHSVMEESQSLNEPSPKQSEEIPEVTSEPVKGSLNRAQSAQSINSTEMPAREDCLKKVSSEPVLSVQEKGVLLKRKLSLLEQDVIVNEDGRNKLKKQGETPNEVCMFSLAYGDIPEELIDVSDFECSLCMRLFFEPVTTPCGHSFCKNCLERCLDHAPYCPLCKESLNEYLADRRYCVTQLLEELIVKYLPDELSERKKIYEEETAELSHLTKNVPIFVCTMAYPTVPCPLHVFEPRYRLMIRRSIQTGTKQFGMCVSDTQNSFADYGCMLQIRNVHFLPDGRSVVDTVGGKRFRVLKRGMKDGYCTADIEYLEDVKVENEDEIKNLRELHDLVYSQACSWFQNLRDRFRSQILQHFGSMPXREENLQAAPNGPAWCWWLLAVLPVDPRYQLSVLSMKSLKERLTKIQHILTYFSRDQSK

SEQ ID NO: 19 - DNA, 310 bp, Homo sapiens
  unsure 300: unknown base
ggatccctggagctgccattagaatcaagaatctttgccaagaactagaagcaaagttttatgaagggacttttaattgggaaagtgtcaaacagcatgatgccgccagcctgctgaagctcttcattcgggagttgccccagccactgctcagtgtggagtatctcaaagcctttcaggctgtccagaatcttccaaccaagaagcagcaactacagggcttttgaaccctttcttttactcattcctgatgtttgatacccctggttgaaaacaattcagtaaagcatcctgcctcanaccccccccc

SEQ ID NO: 20 - DNA, 26 bp, Homo sapiens
gctgcttcttggttggaagattctgg

SEQ ID NO: 21 - DNA, 26 bp, Homo sapiens
ccagaatcttccaaccaagaagcagc

SEQ ID NO: 22 - DNA, 53 bp, Homo sapiens
gcatcatgctgtttgacactttcccaattaaaagtcccttcataaaactttgc

SEQ ID NO: 23 - DNA, 30 bp, Homo sapiens
ggagctgccattagaatcaagaatctttgc

SEQ ID NO: 24 - DNA, 533 bp, Homo sapiens
ttttattaggaatttagcttttattcaaaattaaataagcaaaagcaaaaattttaagaaacttttacaaattacttacataaaagaaagttaataaggacagtcacaaatttgcaacaaataattacaaaagtttctagggcagcatgaatataaaccatgttgcagcatggtgatctaactgtgatatgaataaggcataactaacatttgcaccgagaccagaattaaaaacaaaaacaaactttaaaagcttagttctatattaaacttcttctcttttcccagatccttaatgggtttatactatgcatttttttttaaaacaaacacatcatgtcaaactataaattacacaaatgggcagttaatgtgaaaagccccctaaaatgtacaaactaactggtactgaattgagttctccctttacctttatgtacaattaaatgtaaaccatattttcacagttttgagtgttttatgaatagatgcacagtaccatgtcaggtttacaattgttcctgaaaactggc

SEQ ID NO: 25 - DNA, 433 bp, Homo sapiens
agcagcagcaggaactcgttacgctgcagcagggcccacacggacttggcttccgcccaggacacccggttgtccttgttgaagtcagccatgagcaggacctggccaaccagcgccggcagggaaggcaggtctcccaggttcgccttgaggaagccgagggtcatctcccggaattccttgatggaggtgccccgggtgggcttgtcaaacagtaccagctcccgccgggggccgcatccgaccgggccttggagtcgagggtctcctcaatgccacacttgatggttacatccttgtcccgccagagcccgctgtacacctgctggcccggggcacacgagaagcaggtcctccactccaccatatgcagctcacacaggtcctggcagacggagcccgagatgatccccttgcggtactggtcacaaat

SEQ ID NO: 26 - DNA, 22 bp, Homo sapiens
atgcagttctttttcaacttcc

SEQ ID NO: 27 - DNA, 20 bp, Homo sapiens
ctagagaccaatctaagtaa

SEQ ID NO: 28 - DNA, 20 bp, Homo sapiens
atggtggagtggaggacctg

SEQ ID NO: 29 - DNA, 22 bp, Homo sapiens
ctccaacaccaagtactcttga